(19) United States
(12) Patent Application Publication
(10) Pub. No.: US 2012/0095046 A1
(43) Pub. Date: Apr. 19, 2012

(54) Amido-Isothiazole Compounds and Their Use as Inhibitors of 11Beta-HSD1 for the Treatment of Metabolic Syndrome and Related Disorders

(75) Inventors: Scott Peter Webster, Edinburgh (GB); Jonathan Robert Seckl, Edinburgh (GB); Brian Robert Walker, Edinburgh (GB); Peter Ward, Middlesex (GB); Thomas David Pallin, Essex (GB); Hazel Joan Dyke, Essex (GB); Trevor Robert Perrior, Suffolk (GB)

(73) Assignee: THE UNIVERSITY OF EDINBURGH, Edinburgh (GB)

(21) Appl. No.: 13/377,611

(22) PCT Filed: Jun. 14, 2010
(86) PCT No.: PCT/GB10/01155

§ 371 Date: Dec. 12, 2011

Related U.S. Application Data
(60) Provisional application No. 61/186,963, filed on Jun. 15, 2009.

(51) Int. Cl. A61K 31/4709; C07D 417/14; A61K 31/427; C12N 9/99; A61P 43/00; A61P 3/10; A61P 3/08; A61P 3/04; A61P 3/06; A61P 9/10; A61P 9/00; A61P 3/00; A61P 1/16; A61P 25/00; A61P 25/18; A61P 25/28; A61P 25/16; A61P 1/18; A61P 27/06; A61P 25/22; A61P 25/24; A61P 29/00; A61P 19/10; A61P 9/12; A61P 27/02; A61P 11/06; A61P 11/00; A61P 17/00; A61P 1/00; A61P 19/02; C07D 413/06

(52) U.S. Cl. 514/314; 546/164; 548/214; 514/372; 435/184

(57) ABSTRACT

The present invention pertains generally to the field of therapeutic compounds. More specifically the present invention pertains to certain amido-isothiazole compounds that, inter alia, inhibit 11β-hydroxysteroid dehydrogenase type 1 (11β-HSD1). The present invention also pertains to pharmaceutical compositions comprising such compounds, and the use of such compounds and compositions, both in vitro and in vivo, to inhibit 11β-hydroxysteroid dehydrogenase type 1; to treat disorders that are ameliorated by the inhibition of 11β-hydroxysteroid dehydrogenase type 1; to treat the metabolic syndrome, which includes disorders such as type 2 diabetes and obesity, and associated disorders including insulin resistance, hypertension, lipid disorders and cardiovascular disorders such as ischaemic (coronary) heart disease; to treat CNS disorders such as mild cognitive impairment and early dementia, including Alzheimer's disease; etc.

### RELATED APPLICATION

This application is related to U.S. provisional patent application No. 61/186,963 filed 15 Jun. 2009, the contents of which are incorporated herein by reference in their entirety.

### TECHNICAL FIELD

The present invention pertains generally to the field of therapeutic compounds. More specifically the present invention pertains to certain amido-isothiazole compounds that, inter alia, inhibit 11β-hydroxysteroid dehydrogenase type 1 (11β-HSD1). The present invention also pertains to pharmaceutical compositions comprising such compounds, and the use of such compounds and compositions, both in vitro and in vivo, to inhibit 11β-hydroxysteroid dehydrogenase type 1; to treat disorders that are ameliorated by the inhibition of 11β-hydroxysteroid dehydrogenase type 1; to treat the metabolic syndrome, which includes disorders such as type 2 diabetes and obesity, and associated disorders including insulin resistance, hypertension, lipid disorders and cardiovascular disorders such as ischaemic (coronary) heart disease; to treat CNS disorders such as mild cognitive impairment and early dementia, including Alzheimer's disease; etc.

### BACKGROUND

A number of publications are cited herein in order to more fully describe and disclose the invention and the state of the art to which the invention pertains. Each of these references is incorporated herein by reference in its entirety into the present disclosure, to the same extent as if each individual reference was specifically and individually indicated to be incorporated by reference.

Throughout this specification, including the claims which follow, unless the context requires otherwise, the word “comprise,” and variations such as “comprises” and “comprising,” will be understood to imply the inclusion of a stated integer or step or group of integers or steps but not the exclusion of any other integer or step or group of integers or steps.

It must be noted that, as used in the specification and the appended claims, the singular forms “a,” “an,” and “the” include plural referents unless the context clearly dictates otherwise. Thus, for example, reference to “a pharmaceutical carrier” includes mixtures of two or more such carriers, and the like.

Ranges are often expressed herein as from “about” one particular value, and/or to “about” another particular value. When such a range is expressed, another embodiment includes from the one particular value and/or to the other particular value. Similarly, when values are expressed as approximations, by the use of the antecedent “about,” it will be understood that the particular value forms another embodiment.

This disclosure includes information that may be useful in understanding the present invention. It is not an admission that any of the information provided herein is prior art or relevant to the presently claimed invention, or that any publication specifically or implicitly referenced is prior art.

Glucocorticoids (cortisol in man, corticosterone in rodents) are hormones that regulate a range of pathways involved in stress and metabolic signalling. They are antagonists of insulin action and impair insulin-dependent glucose uptake, increase lipolysis, and enhance hepatic gluconeogenesis. These effects are evident in Cushing's syndrome, which is caused by elevated circulating levels of glucocorticoids. The features of Cushing's syndrome are diverse and reflect the tissue distribution of glucocorticoid receptors in the body. They include a cluster of metabolic (central/visceral obesity, insulin resistance, hyperglycaemia, dyslipidaemia) and cardiovascular (hypertension) abnormalities which, when observed in patients without Cushing's syndrome, constitute the metabolic syndrome. These abnormalities confer a substantial risk of cardiovascular disease. In addition, Cushing's syndrome is associated with neuropsychiatric manifestations including depression and cognitive impairment. The features of Cushing's syndrome are reversible upon removal of the cause of glucocorticoid excess.

It is recognised that glucocorticoid activity is controlled at the tissue level by the intracellular conversion of active cortisol and inactive cortisone by 11β-hydroxysteroid dehydrogenases (see, e.g., Seckl et al., 2001). These enzymes exist in two distinct isoforms. 11β-HSD1, which catalyses the reaction that activates cortisone, is expressed in liver, adipose tissue, brain, skeletal muscle, vascular smooth muscle and other organs, while, 11β-HSD2, which inactivates cortisol, is predominantly expressed in the kidney. Pharmacological inhibition of 11β-HSD1 in rat and man with carbenoxolone (see, e.g., Walker et al., 1995), and transgenic knockout in mice (see, e.g., Kotelevtsev et al., 1997), results in enhanced hepatic insulin sensitivity and reduced gluconeogenesis and glycogenolysis, suggesting that 11β-HSD1 inhibition will be a useful treatment in type 2 diabetes and other insulin resistance syndromes. Furthermore, mice lacking 11β-HSD1 possess low triglycerides, increased HDL cholesterol, and increased apo-lipoprotein A-I levels (see, e.g., Morton et al., 2001), suggesting that inhibitors of 11β-HSD1 may be of utility in the treatment of atherosclerosis.

The link between 11β-HSD1 and the metabolic syndrome has been strengthened by studies in transgenic mice and man. 11β-HSD1 knockout mice on two different genetic backgrounds are protected from dietary obesity (see, e.g., Morton et al., 2004), while administration of carbenoxolone to patients with type 2 diabetes enhances insulin sensitivity (see, e.g., Andrews et al., 2003). However, it has become apparent that the key tissue in which 11β-HSD1 exerts the greatest influence upon metabolic disease is the adipose tissue rather than the liver. Mice with transgenic overexpression of 11β-HSD1 in adipose tissue (see, e.g. Masuzaki et al., 2001) have a more profound metabolic syndrome and obesity than mice with overexpression in liver (see, e.g., Paterson et al., 2004). In obese humans, 11β-HSD1 activity is increased in adipose tissue, but enzyme activity is decreased in the liver (see, e.g., Rask et al., 2001).

In the CNS, 11β-HSD1 is highly expressed in regions important for cognition such as hippocampus, frontal cortex, and cerebellum (see, e.g., Moisan et al., 1990). Elevated cortisol is associated with cognitive dysfunction, and glucocorticoids have a range of neurotoxic effects. 11β-HSD1 knockout mice are protected against age-related cognitive dysfunction (see, e.g., Yau et al., 2001), while administration of the 11β-HSD inhibitor carbenoxolone has been shown to enhance cognitive function in elderly men and type 2 diabetics who have a selective impairment in verbal memory (see, e.g., Sandeep et al., 2004). Thus, 11β-HSD1 inhibitors are of potential therapeutic utility in the treatment of diseases such as Alzheimer's Disease, which are characterised by cognitive impairment.

The isozymes of 11β-HSD are also expressed in the blood vessel wall (see, e.g., Walker et al., 1991; Christy et al., 2003). 11β-HSD1 is expressed in vascular smooth muscle, while 11β-HSD2 is expressed in endothelial cells where it modulates endothelial-dependent vasodilation (see, e.g., Hadoke et al., 2001). 11β-HSD1 knockout mice have normal vascular function, but they exhibit enhanced angiogenesis in response to inflammation or ischaemia (see, e.g., Small et al., 2005). This offers therapeutic potential in the treatment of myocardial infarction, since inhibition of 11β-HSD1 may enhance revascularisation of ischaemic tissues.

Studies have shown that 11β-HSD1 affects intraocular pressure in man (see, e.g., Rauz et al., 2001). Inhibition of 11β-HSD1 may be useful in reducing intraocular pressure in the treatment of glaucoma.

Glucocorticoids are involved in the regulation of bone formation and skeletal development. Treatment of healthy volunteers with carbenoxolone led to a decrease in bone resorption markers suggesting that 11β-HSD1 plays a role in bone resorption (see, e.g., Cooper et al., 2000). 11β-HSD1 inhibitors could be used as protective agents in the treatment of osteoporosis.

The inventors have discovered compounds that inhibit 11β-hydroxysteroid dehydrogenase type 1 (11β-HSD1) that are useful in the treatment, control, and/or prevention of disorders (e.g., diseases) that are responsive to the inhibiton of 11β-HSD1.

### SUMMARY OF THE INVENTION

One aspect of the invention pertains to certain amido-isothiazoles (referred to herein as AITZ compounds), as described herein.

Another aspect of the invention pertains to a composition (e.g., a pharmaceutical composition) comprising an AITZ compound, as described herein, and a pharmaceutically acceptable carrier or diluent.

Another aspect of the invention pertains to a method of preparing a composition (e.g., a pharmaceutical composition) comprising the step of admixing an AITZ compound, as described herein, and a pharmaceutically acceptable carrier or diluent.

Another aspect of the present invention pertains to a method of inhibiting 11β-hydroxysteroid dehydrogenase type 1 (11β-HSD1) function (e.g., in a cell), in vitro or in vivo, comprising contacting the cell with an effective amount of an AITZ compound, as described herein.

Another aspect of the present invention pertains to a method of treatment comprising administering to a subject in need of treatment a therapeutically-effective amount of an AITZ compound, as described herein, preferably in the form of a pharmaceutical composition.

Another aspect of the present invention pertains to an AITZ compound as described herein for use in a method of treatment of the human or animal body by therapy.

Another aspect of the present invention pertains to use of an AITZ compound, as described herein, in the manufacture of a medicament for use in treatment.

In one embodiment, the treatment is treatment or prevention of a disorder (e.g., a disease) that is ameliorated by the inhibition of 11β-hydroxysteroid dehydrogenase type 1 (11β-HSD1).

In one embodiment, the treatment is treatment or prevention of metabolic syndrome, which includes conditions such as type 2 diabetes and obesity, and associated disorders including insulin resistance, hypertension, lipid disorders and cardiovascular disorders such as ischaemic (coronary) heart disease.

In one embodiment, the treatment is treatment or prevention of a CNS disorder (e.g., a CNS disease) such as mild cognitive impairment and early dementia, including Alzheimer's disease.

Another aspect of the present invention pertains to a kit comprising (a) an AITZ compound, as described herein, preferably provided as a pharmaceutical composition and in a suitable container and/or with suitable packaging; and (b) instructions for use, for example, written instructions on how to administer the compound.

Another aspect of the present invention pertains to an AITZ compound obtainable by a method of synthesis as described herein, or a method comprising a method of synthesis as described herein.

Another aspect of the present invention pertains to an AITZ compound obtained by a method of synthesis as described herein, or a method comprising a method of synthesis as described herein.

Another aspect of the present invention pertains to novel intermediates, as described herein, which are suitable for use in the methods of synthesis described herein.

Another aspect of the present invention pertains to the use of such novel intermediates, as described herein, in the methods of synthesis described herein.

As will be appreciated by one of skill in the art, features and preferred embodiments of one aspect of the invention will also pertain to other aspects of the invention.

### DETAILED DESCRIPTION OF THE INVENTION

### Compounds

One aspect of the present invention relates to certain amido-isothiazoles (for convenience, collectively referred to herein as “amido-isothiazole compounds” or “AITZ compounds”).

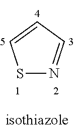

In one embodiment, the compounds are selected from compounds of the following formulae, and pharmaceutically acceptable salts, hydrates, and solvates thereof:

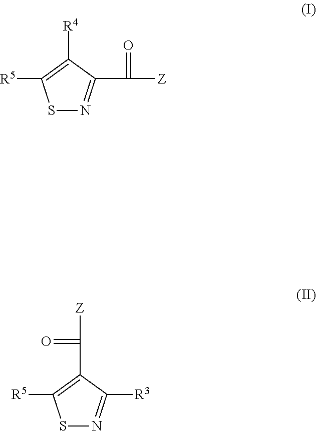

wherein:
—R³ is independently —H, —R^{3A}, or —R^{3B};
—R⁴ is independently —H, —R^{4A}, or —R^{4B};
—R⁵ is independently —R^{5A1}, —R^{5A2}, —R^{5B1}, or —R^{5B2};
—Z is independently -J¹ or -J²;
   wherein:
—R^{3A} is independently saturated aliphatic C₁₋₄alkyl;
—R^{3B} is independently —F, —Cl or —Br;
—R^{4A} is independently saturated aliphatic C₁₋₄alkyl;
—R^{4B} is independently —F, —Cl or —Br;
—R^{5A1} is independently phenyl or naphthyl, and is optionally substituted;
—R^{5A2} is independently C₃₋₇cycloalkyl, and is optionally substituted;
—R^{5B1} is independently C₅₋₁₀heteroaryl, and is optionally substituted;
—R^{5B2} is independently non-aromatic C₄₋₇heterocyclyl, and is optionally substituted;
-J¹ is independently a monocyclic non-aromatic heterocyclyl group having from 4 to 8 ring atoms, wherein exactly 1 of said ring atoms is a ring heteroatom, and is N, or exactly 2 of said ring atoms are ring heteroatoms, and are both N, or exactly 2 of said ring atoms are ring heteroatoms, and are N and O, or exactly 2 of said ring atoms are ring heteroatoms, and are N and S, and wherein said non-aromatic heterocyclyl group is optionally substituted, and wherein -J¹ is attached via one of said ring atoms which is N; and
-J² is independently a fused bicyclic non-aromatic heterocyclyl group having from 7 to 12 ring atoms, wherein exactly 1 of said ring atoms is a ring heteroatom, and is N, or exactly 2 of said ring atoms are ring heteroatoms, and are both N, or exactly 2 of said ring atoms are ring heteroatoms, and are N and O, or exactly 2 of said ring atoms are ring heteroatoms, and are N and S, or exactly 3 of said ring atoms are ring heteroatoms, one of which is N, and each of the other two is independently N, O, or S, and wherein said fused bicyclic non-aromatic heterocyclyl group is optionally substituted, and wherein -J¹ is attached via one of said ring atoms which is N.

For the avoidance of doubt, it is not intended that —R⁴ and —R⁵ or —R³ and —R⁵ are attached to one another other than as shown in the above formulae. For example, it is not intended that —R⁴ and —R⁵ together form a ring fused to the central isothiazole ring. Similarly, it is not intended that —R³ and —R⁵ together form a ring fused to the central isothiazole ring.

Also for the avoidance of doubt, it is not intended that —R⁴ and —R⁵ are attached to —Z, or that —R³ and —R⁵ are attached to —Z, other than as shown in the above formulae. For example, it is not intended that —R³ and —Z together form a ring fused to the central isothiazole ring. Similarly, it is not intended that —R⁴ and —Z together form a ring fused to the central isothiazole ring. Similarly, it is not intended that —R⁵ and —Z together form a ring fused to the central isothiazole ring.

### Optional Provisos

In one or more aspects of the present invention (e.g., compounds, compositions, compounds for use in therapy, use of compounds in the manufacture of a medicament, methods, methods of treatment, etc.), the compounds are optionally as defined herein, but with one or more optional provisos, as defined herein.

In one embodiment, the proviso is that the compound is not a compound selected from: compound (PP-01) and salts, hydrates, and solvates thereof.

| | | | Registry |
|---|---|---|---|
| # | Structure | Name | No. |
| PP-01 | 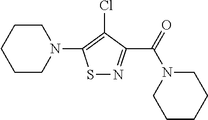 | (4-Chloro-5- piperidin-1-yl- isothiazol-3-yl)- piperidin- 1-yl-methanone | 799262-71-8 |

In one or more aspects of the present invention (e.g., compounds for use in therapy, use of compounds in the manufacture of a medicament, methods of treatment, etc.), the compounds are optionally as defined herein, but without the proviso regarding compound (PP-01).

For example, a reference to a particular group of compounds “without the recited proviso regarding compound (PP-01)” (e.g., for use in therapy) is intended to be a reference to the compounds as defined, but wherein the definition no longer includes the indicated proviso. In such cases, it is as if the indicated proviso has been deleted from the definition of compounds, and the definition has been expanded to encompass those compounds which otherwise would have been excluded by the indicated proviso.

In one or more aspects of the present invention (e.g., compounds for use in therapy, use of compounds in the manufacture of a medicament, methods of treatment, etc.), the compounds are optionally as defined herein, with the proviso regarding compound (PP-01).

### Formula (I) and Formula (II)

In one embodiment, the compounds are selected from compounds of the following formula, and pharmaceutically acceptable salts, hydrates, and solvates thereof, wherein —R⁴, —R⁵, and —Z are as defined herein:

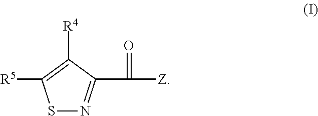

In one embodiment, the compounds are selected from compounds of the following formula, and pharmaceutically acceptable salts, hydrates, and solvates thereof, wherein —R³, —R⁵, and —Z are as defined herein:

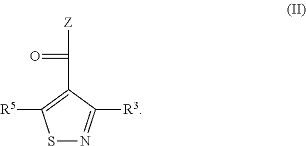

### The Group —R⁵

In one embodiment, —R⁵ is independently —R^{5A1}, —R^{5A2}, —R^{5B1}, or —R^{5B2}.

In one embodiment, —R⁵ is independently —R^{5A1}, —R^{5A2} or —R^{5B1}.

In one embodiment, —R⁵ is independently —R^{5A1}, —R^{5B1}, or —R^{5B2}.

In one embodiment, —R⁵ is independently —R^{5A1} or —R^{5A2}.

In one embodiment, —R⁵ is independently —R^{5B1} or —R^{5B2}.

In one embodiment, —R⁵ is independently —R^{5A1} or —R^{5B1}.

In one embodiment, —R⁵ is independently —R^{5A2} or —R^{5B2}.

In one embodiment, —R⁵ is independently —R^{5A1}.

In one embodiment, —R⁵ is independently —R^{5A2}.

In one embodiment, —R⁵ is independently —R^{5B1}.

In one embodiment, —R⁵ is independently —R^{5B2}.

The Group —Z

In one embodiment, —Z is independently J¹ or -J².

In one embodiment, —Z is independently -J¹.

In one embodiment, —Z is independently -J².

The Group —R³

In one embodiment, —R³, if present, is independently —H, —R^{3A} or —R³⁶.

In one embodiment, —R³, if present, is independently —H or —R^{3A}.

In one embodiment, —R³, if present, is independently —H or —R³⁸.

In one embodiment, —R³, if present, is independently —R^{3A} or —R³⁸.

In one embodiment, —R³, if present, is independently —H.

In one embodiment, —R³, if present, is independently —R^{3A}.

In one embodiment, —R³, if present, is independently —R³⁸.

### The Group —R⁴

In one embodiment, —R⁴, if present, is independently —H, —R^{4A} or —R^{4B}.

In one embodiment, —R⁴, if present, is independently —H or —R^{4A}.

In one embodiment, —R⁴, if present, is independently —H or —R^{4B}.

In one embodiment, —R⁴, if present, is independently —R^{4A} or —R^{4B}.

In one embodiment, —R⁴, if present, is independently —H.

In one embodiment, —R⁴, if present, is independently —R^{4A}.

In one embodiment, —R⁴, if present, is independently —R^{4B}.

### The Group —R^{5A1}

In one embodiment, —R^{5A1}, if present, is independently phenyl or naphthyl, and is optionally substituted.

In one embodiment, —R^{5A1}, if present, is independently phenyl, and is optionally substituted.

In one embodiment, —R^{5A1}, if present, is independently selected from the groups —R^{5A1} shown in the compounds described under the heading “Examples of Specific Embodiments”.

The Group —R^{5A2}

In one embodiment, —R^{5A2}, if present, is independently C₃₋₇cycloalkyl, and is optionally substituted.

In one embodiment, —R^{5A2}, if present, is independently cyclopropyl, cyclobutyl, cyclopentyl, or cyclohexyl, and is optionally substituted.

In one embodiment, —R^{5A2}, if present, is independently selected from the groups —R^{5A2} shown in the compounds described under the heading “Examples of Specific Embodiments”.

The Group —R^{5B1}

In one embodiment, —R^{5B1}, if present, is independently C₅₋₁₀heteroaryl, and is optionally substituted.

In one embodiment, —R^{5B1}, if present, is independently furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, indolyl, isoindolyl, benzofuranyl, isobenzofuranyl, benzothienyl, isobenzothienyl, indazolyl, benzimidazolyl, benzothiazolyl, benzoxazolyl, quinolinyl, isoquinolinyl, cinnolinyl, or quinazolinyl, and is optionally substituted.

In one embodiment, —R^{5B1}, if present, is independently C₅₋₆heteroaryl, and is optionally substituted.

In one embodiment, —R^{5B1}, if present, is independently furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, pyridazinyl, and is optionally substituted.

In one embodiment, —R^{5B1}, if present, is independently imidazolyl, pyrazolyl, oxazolyl, isoxazolyl, pyridyl, pyrimidinyl, or quinolinyl, and is optionally substituted.

In one embodiment, —R^{5B1}, if present, is independently pyrazolyl, and is optionally substituted.

In one embodiment, —R^{5B1}, if present, is independently pyrazol-1-yl, and is optionally substituted.

In one embodiment, —R^{5B1}, if present, is independently pyrazol-3-yl, and is optionally substituted.

In one embodiment, —R^{5B1}, if present, is independently pyrazol-4-yl, and is optionally substituted.

In one embodiment, —R^{5B1}, if present, is independently oxazolyl, and is optionally substituted.

In one embodiment, —R^{5B1}, if present, is independently oxazol-2-yl, and is optionally substituted.

In one embodiment, —R^{5B1}, if present, is independently oxazol-4-yl, and is optionally substituted.

In one embodiment, —R^{5B1}, if present, is independently isoxazolyl, and is optionally substituted.

In one embodiment, —R^{5B1}, if present, is independently isoxazol-4-yl, and is optionally substituted.

In one embodiment, —R^{5B1}, if present, is independently pyridyl, and is optionally substituted.

In one embodiment, —R^{5B1}, if present, is independently pyrid-2-yl, and is optionally substituted.

In one embodiment, —R^{5B1}, if present, is independently pyrid-3-yl, and is optionally substituted.

In one embodiment, —R^{5B1}, if present, is independently pyrid-4-yl, and is optionally substituted.

In one embodiment, —R^{5B1}, if present, is independently pyrimidinyl, and is optionally substituted.

In one embodiment, —R^{5B1}, if present, is independently pyrimidin-5-yl, and is optionally substituted.

In one embodiment, —R^{5B1}, if present, is independently quinolinyl, and is optionally substituted.

In one embodiment, —R^{5B1}, if present, is independently quinolin-6-yl, and is optionally substituted.

In one embodiment, —R^{5B1}, if present, is independently furanyl, and is optionally substituted.

In one embodiment, —R^{5B1}, if present, is independently thienyl, and is optionally substituted.

In one embodiment, —R^{5B1}, if present, is independently pyrrolyl, and is optionally substituted.

In one embodiment, —R^{5B1}, if present, is independently triazolyl, and is optionally substituted.

In one embodiment, —R^{5B1}, if present, is independently tetrazolyl, and is optionally substituted.

In one embodiment, —R^{5B1}, if present, is independently thiazolyl, and is optionally substituted.

In one embodiment, —R^{5B1}, if present, is independently isothiazolyl, and is optionally substituted.

In one embodiment, —R^{5B1}, if present, is independently pyridazinyl, and is optionally substituted.

In one embodiment, —R^{5B1}, if present, is independently indolyl, and is optionally substituted.

In one embodiment, —R^{5B1}, if present, is independently isoindolyl, and is optionally substituted.

In one embodiment, —R^{5B1}, if present, is independently benzofuranyl, and is optionally substituted.

In one embodiment, —R^{5B1}, if present, is independently isobenzofuranyl, and is optionally substituted.

In one embodiment, —R^{5B1}, if present, is independently benzothienyl, and is optionally substituted.

In one embodiment, —R^{5B1}, if present, is independently isobenzothienyl, and is optionally substituted.

In one embodiment, —R^{5B1}, if present, is independently indazolyl, and is optionally substituted.

In one embodiment, —R^{5B1}, if present, is independently benzimidazolyl, and is optionally substituted.

In one embodiment, —R^{5B1}, if present, is independently benzothiazolyl, and is optionally substituted.

In one embodiment, —R^{5B1}, if present, is independently benzoxazolyl, and is optionally substituted.

In one embodiment, —R^{5B1}, if present, is independently isoquinolinyl, and is optionally substituted.

In one embodiment, —R^{5B1}, if present, is independently cinnolinyl, and is optionally substituted.

In one embodiment, —R^{5B1}, if present, is independently quinazolinyl, and is optionally substituted.

In one embodiment, —R^{5B1}, if present, is independently selected from the groups —R^{5B1} shown in the compounds described under the heading “Examples of Specific Embodiments”.

The Group —R^{5B2}

In one embodiment, —R^{5B2}, if present, is independently non-aromatic C₄₋₇heterocyclyl, and is optionally substituted.

In one embodiment, —R^{5B2} is independently azetidinyl, oxitanyl, pyrrolidinyl, tetrahydrofuranyl, piperidinyl, tetrahydropyridinyl, tetrahydropyranyl, piperazinyl, morpholinyl, thiomorpholinyl, tetrahydrothiopyranyl, tetrahydrothiopyran-1,1-dioxide, azepanyl, diazepanyl, or oxazepanyl, and is optionally substituted.

In one embodiment, —R^{5B2} is independently azetidinyl, oxitanyl, pyrrolidinyl, tetrahydrofuranyl, piperidinyl, tetrahydropyridinyl, tetrahydropyranyl, piperazinyl, morpholinyl, thiomorpholinyl, azepanyl, diazepanyl, or oxazepanyl, and is optionally substituted.

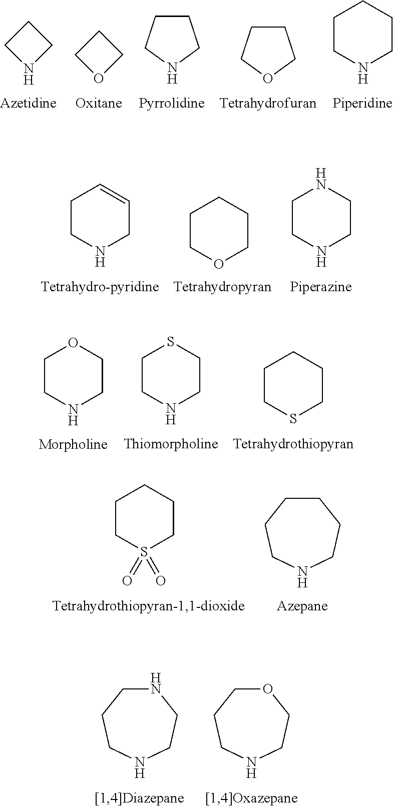

In one embodiment, —R^{5B2} is independently tetrahydropyranyl, and is optionally substituted.

In one embodiment, —R^{5B2} is independently tetrahydropyran-4-yl, and is optionally substituted.

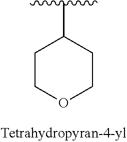

In one embodiment, —R^{5B2} is independently piperidinyl, and is optionally substituted.

In one embodiment, —R^{5B2} is independently piperidin-1-yl, and is optionally substituted.

In one embodiment, —R^{5B2} is independently as defined herein, but is not piperidino (i.e., piperidin-1-yl), that is, —R^{5B2} is not piperidin-1-yl.

In one embodiment, —R^{5B2} is independently piperidin-3-yl, and is optionally substituted.

In one embodiment, —R^{5B2} is independently piperidin-2-yl, and is optionally substituted.

In one embodiment, —R^{5B2} is independently piperidin-4-yl, and is optionally substituted.

In one embodiment, —R^{5B2} is independently piperidin-2-one-4-yl, and is optionally substituted.

In one embodiment, —R^{5B2} is independently piperidin-2-one-5-yl, and is optionally substituted.

In one embodiment, —R^{5B2} is independently piperidin-2-one-6-yl, and is optionally substituted.

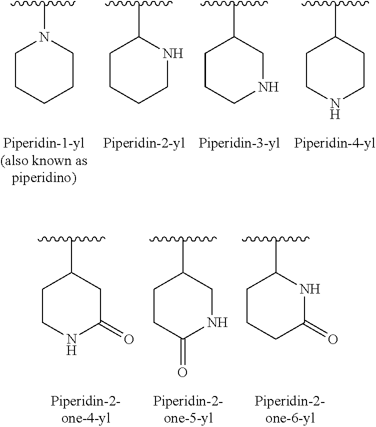

For example, in one embodiment, —R^{5B2} is independently piperidin-4-yl, and is optionally N-substituted, or is N-substituted, for example, with a substituent as defined herein (see “Optional Substituents on the Groups —R^{5A1}, —R^{5B1}, —R^{5A2}, and —R^{5B2}”).

For example, in one embodiment, —R^{5B2} is independently selected from:

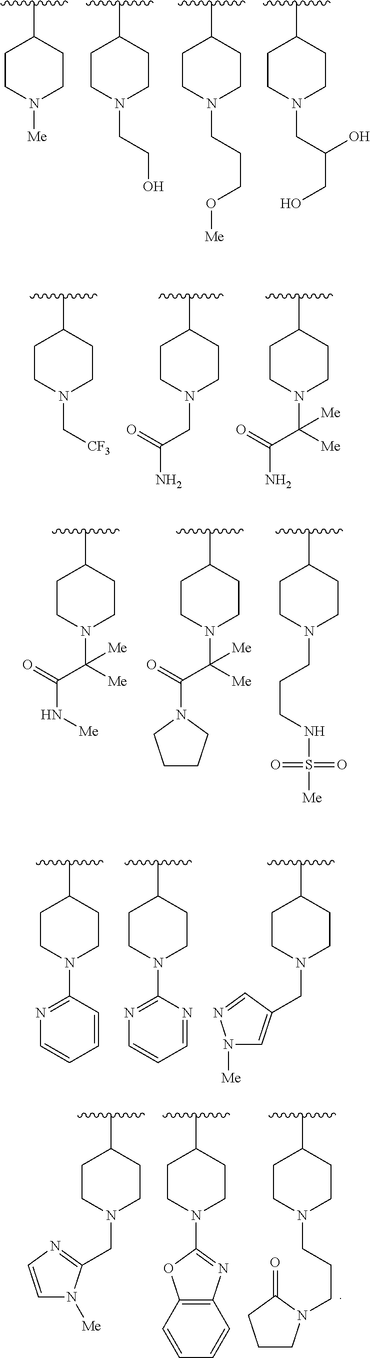

For example, in one embodiment, —R^{5B2} is independently selected from:

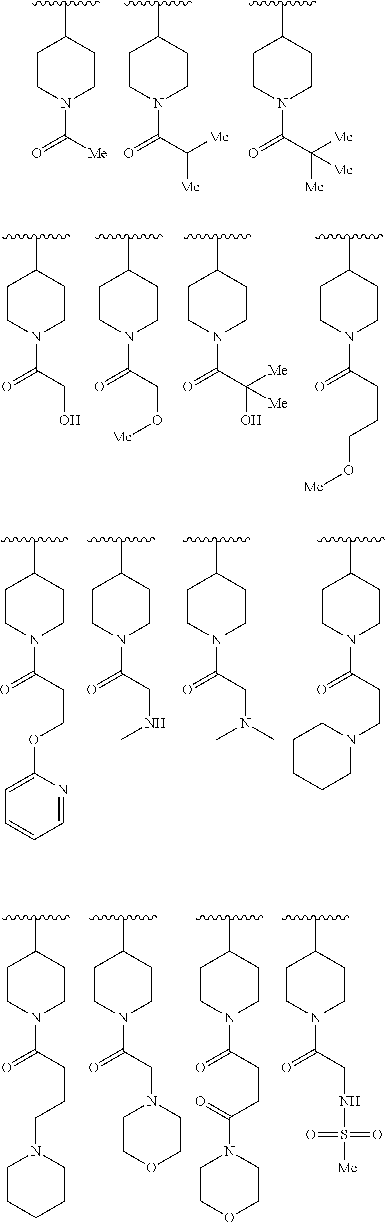

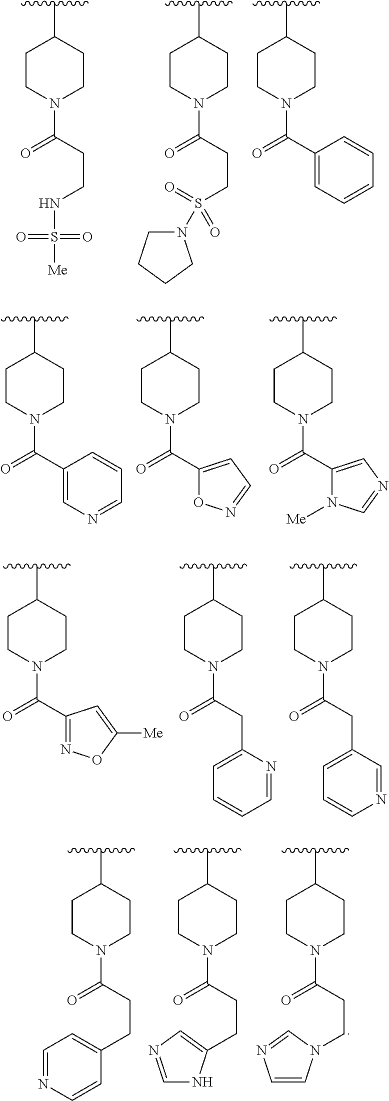

For example, in one embodiment, —R^{5B2} is independently selected from:

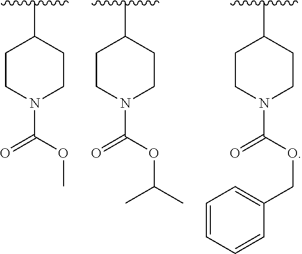

For example, in one embodiment, —R^{5B2} is independently selected from:

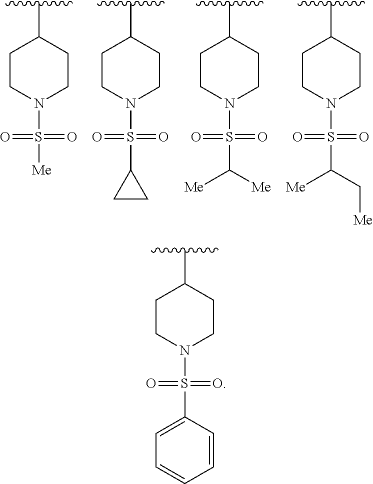

For example, in one embodiment, —R^{5B2} is independently selected from:

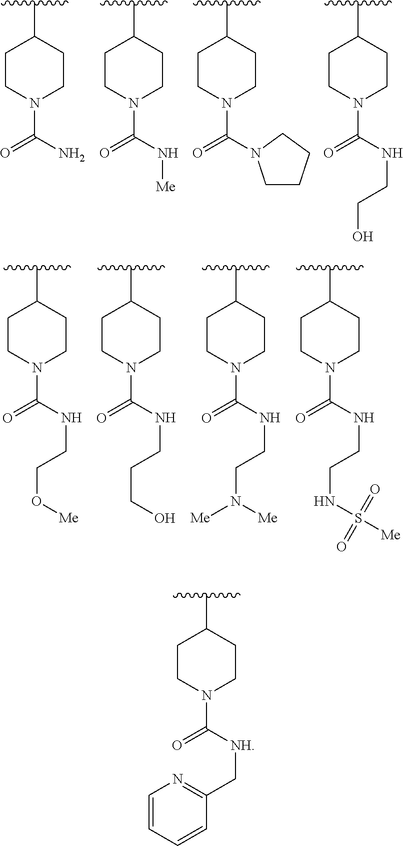

In one embodiment, —R^{5B2} is independently tetrahydropyridinyl, and is optionally substituted.

In one embodiment, —R^{5B2} is independently 1,2,3,6-tetrahydropyridin-4-yl, and is optionally substituted.

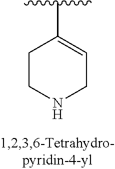

For example, in one embodiment, —R^{5B2} is independently 1,2,3,6-tetrahydropyridin-4-yl, and is N-substituted, for example, with a substituent as defined herein (see “Optional Substituents on the Groups —R^{5A1}, —R^{5B1}, —R^{5A2}, and —R^{5B2}”).

For example, in one embodiment, —R^{5B2} is independently selected from:

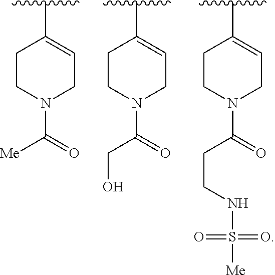

In one embodiment, —R^{5B2} is independently piperazinyl, and is optionally substituted.

In one embodiment, —R^{5B2} is independently piperazin-1-yl, and is optionally substituted.

In one embodiment, —R^{5B2} is independently piperazin-2-one-1-yl, and is optionally substituted.

In one embodiment, —R^{5B2} is independently piperazin-3-one-1-yl, and is optionally substituted.

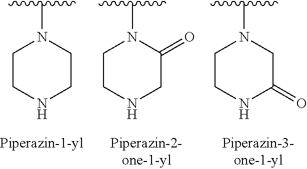

In one embodiment, —R^{5B2} is independently pyrrolidinyl, and is optionally substituted.

In one embodiment, —R^{5B2} is independently pyrrolidin-1-yl, and is optionally substituted.

In one embodiment, —R^{5B2} is independently pyrrolidin-2-one-1-yl, and is optionally substituted.

In one embodiment, —R^{5B2} is independently pyrrolidin-3-yl, and is optionally substituted.

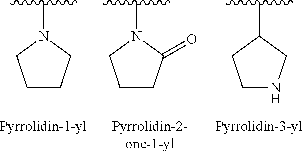

For example, in one embodiment, —R^{5B2} is independently pyrrolidin-3-yl, and is N-substituted, for example, with a substituent as defined herein (see “Optional Substituents on the Groups —R^{5A1}, —R^{5B1}, —R^{5A2}, and —R^{5B2}”).

For example, in one embodiment, —R^{5B2} is independently:

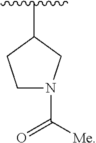

In one embodiment, —R^{5B2} is independently azetidinyl, and is optionally substituted.

In one embodiment, —R^{5B2} is independently azetidin-1-yl, and is optionally substituted.

In one embodiment, —R^{5B2} is independently azetidin-3-yl, and is optionally substituted.

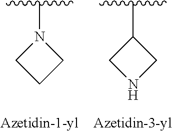

In one embodiment, —R^{5B2} is independently morpholinyl, and is optionally substituted.

In one embodiment, —R^{5B2} is independently morpholin-4-yl, and is optionally substituted.

In one embodiment, —R^{5B2} is independently morpholin-3-one-4-yl, and is optionally substituted.

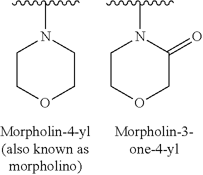

In one embodiment, —R^{5B2} is independently thiomorpholinyl, and is optionally substituted.

In one embodiment, —R^{5B2} is independently thiomorpholin-4-yl, and is optionally substituted.

In one embodiment, —R^{5B2} is independently thiomorpholin-3-one-4-yl, and is optionally substituted.

In one embodiment, —R^{5B2} is independently thiomorpholin-1,1-dioxide-4-yl, and is optionally substituted.

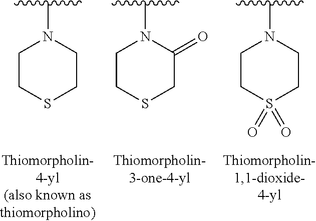

In one embodiment, —R^{5B2}, if present, is independently selected from the groups —R^{5B2} shown in the compounds described under the heading “Examples of Specific Embodiments”.

### The Group —R^{3A}

In one embodiment, —R^{3A}, if present, is independently saturated aliphatic C₁₋₄alkyl.

In one embodiment, —R^{3A}, if present, is independently -Me, -Et, -nPr, or -iPr.

In one embodiment, —R^{3A}, if present, is independently -Me.

### The Group —R^{3B}

In one embodiment, —R^{3B}, if present, is independently —F, —Cl, or —Br.

In one embodiment, —R^{3B}, if present, is independently —Cl or —Br.

In one embodiment, —R^{3B}, if present, is independently —Cl.

In one embodiment, —R^{3B}, if present, is independently —Br.

The Group —R^{4A}

In one embodiment, —R^{4A}, if present, is independently saturated aliphatic C₁₋₄alkyl.

In one embodiment, —R^{4A}, if present, is independently -Me, -Et, -nPr, or -iPr.

In one embodiment, —R^{4A}, if present, is independently -Me.

The Group —R^{4B}

In one embodiment, —R^{4B}, if present, is independently —F, —Cl, or —Br.

In one embodiment, —R^{4B}, if present, is independently —Cl or —Br.

In one embodiment, —R^{4B}, if present, is independently —Cl.

In one embodiment, —R^{4B}, if present, is independently —Br.

The Group -J¹

In one embodiment, -J¹, if present, is independently a monocyclic non-aromatic heterocyclyl group having from 4 to 8 ring atoms, wherein exactly 1 of said ring atoms is a ring heteroatom, and is N, or exactly 2 of said ring atoms are ring heteroatoms, and are both N, or exactly 2 of said ring atoms are ring heteroatoms, and are N and O, or exactly 2 of said ring atoms are ring heteroatoms, and are N and S, and wherein said non-aromatic heterocyclyl group is optionally substituted, for example, with one or more substituents as discussed below under the heading “Optional Substituents on -J¹ and -J²”, and wherein -J¹ is attached via one of said ring atoms which is N.

For the avoidance of doubt, when -J¹ is attached (i.e., to the carbonyl group shown in Formulae (I) and (II)) via one of said ring atoms which is N, the resulting compound is an amide.

For the avoidance of doubt, it is not intended that substituents on -J¹ form a ring fused to said monocyclic non-aromatic heterocyclyl group; that is, substituents on -J¹, if present, do not form a ring fused to said monocyclic non-aromatic heterocyclyl group. For example, it is not intended that -J¹ encompass benzazepinyl. However, it is intended that -J¹ may bear, for example, a substitutent that is or comprises a ring, for example, a substituent that is phenyl.

In one embodiment, exactly 1 of said -J¹ ring atoms is a ring heteroatom, and is N.

In one embodiment, exactly 2 of said -J¹ ring atoms are ring heteroatoms, and are both N.

In one embodiment, exactly 2 of said -J¹ ring atoms are ring heteroatoms, and are N and O.

In one embodiment, exactly 2 of said -J¹ ring atoms are ring heteroatoms, and are N and S.

In one embodiment, said -J¹ monocyclic non-aromatic heterocyclyl group has from 4 to 7 ring atoms.

In one embodiment, said -J¹ monocyclic non-aromatic heterocyclyl group has from 5 to 7 ring atoms.

In one embodiment, said -J¹ monocyclic non-aromatic heterocyclyl group has 6 or 7 ring atoms.

In one embodiment, -J¹, if present, is independently selected from the following groups and is optionally substituted, for example, with one or more substituents as discussed below under the heading “Optional Substituents on -J¹ and -J²”, for example, one or more substituents selected from saturated aliphatic C₁₋₄alkyl:

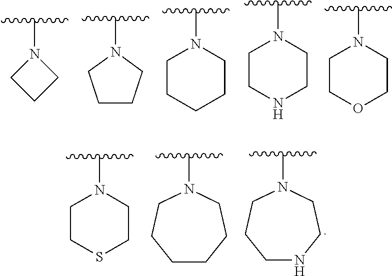

In one embodiment, -J¹, if present, is independently selected from the following groups and is optionally substituted, for example, with one or more substituents as discussed below under the heading “Optional Substituents on -J¹ and -J²”, for example, with one or more substituents selected from saturated aliphatic C₁₋₄alkyl:

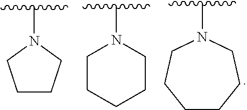

In one embodiment, -J¹, if present, is independently the following group and is optionally substituted, for example, with one or more substituents as discussed below under the heading “Optional Substituents on -J¹ and -J²”, for example, one or more substituents selected from saturated aliphatic C₁₋₄alkyl:

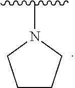

In one embodiment, -J¹, if present, is independently:

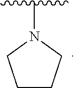

In one embodiment, -J¹, if present, is independently selected from the following group and is optionally substituted, for example, with one or more substituents as discussed below under the heading “Optional Substituents on -J¹ and -J²”, for example, one or more substituents selected from saturated aliphatic C₁₋₄alkyl:

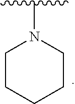

In one embodiment, -J¹, if present, is independently:

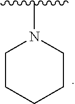

In one embodiment, -J¹, if present, is independently the following group and is optionally substituted, for example, with one or more substituents as discussed below under the heading “Optional Substituents on -J¹ and -J²”, for example, one or more substituents selected from saturated aliphatic C₁₋₄alkyl:

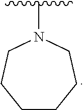

In one embodiment, -J¹, if present, is independently:

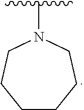

In one embodiment, -J¹, if present, is independently selected from the following groups and is optionally substituted, for example, with one or more substituents as discussed below under the heading “Optional Substituents on -J¹ and -J²”, for example, one or more substituents selected from saturated aliphatic C₁₋₄alkyl:

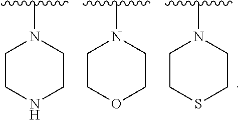

Examples of -J¹ groups (e.g., wherein exactly 1 of said ring atoms is a ring heteroatom, and is N) which additionally bear one or more substituents include the following:

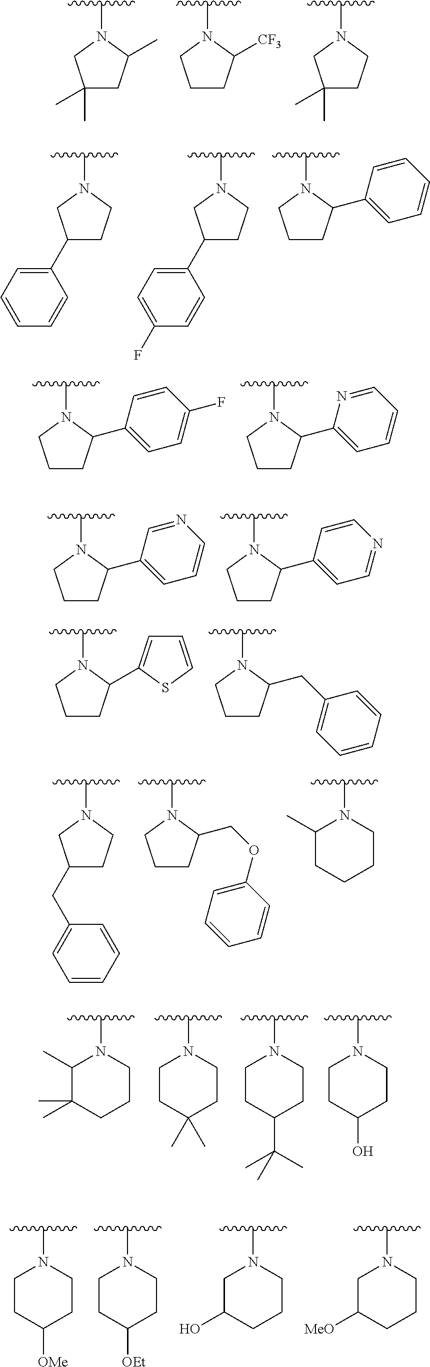

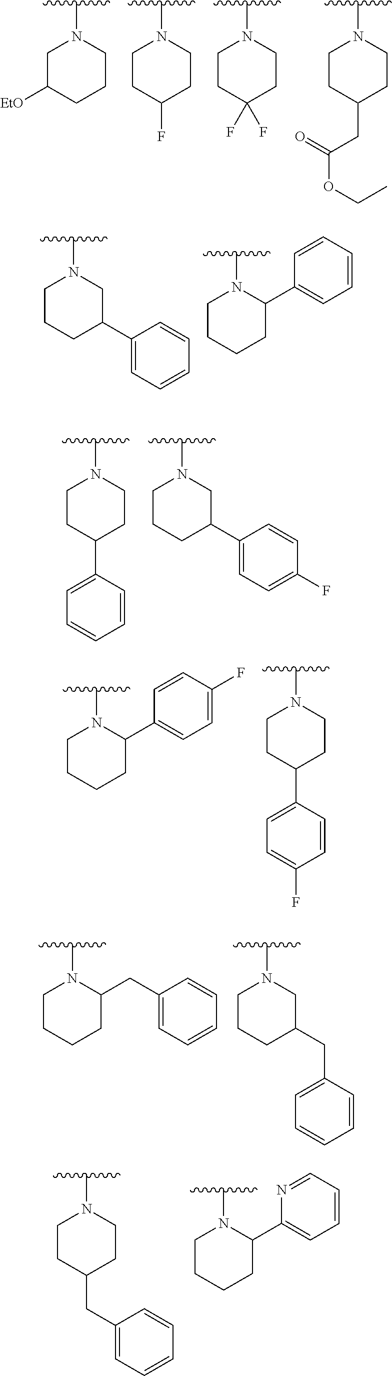

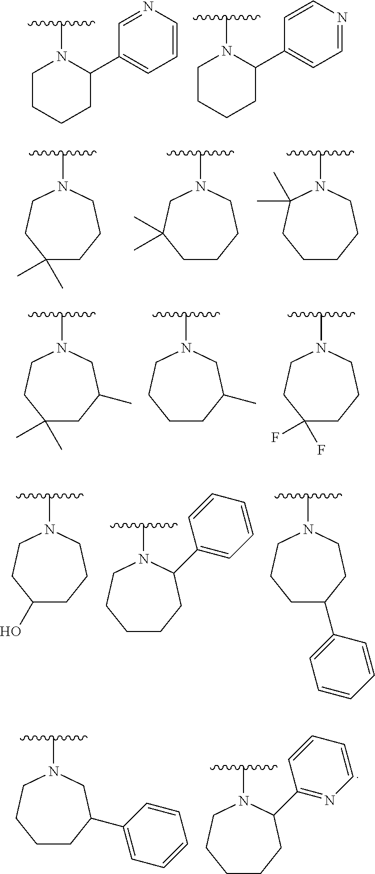

Examples of -J¹ groups (wherein exactly 2 of said ring atoms are ring heteroatoms, and are both N; or wherein exactly 2 of said ring atoms are ring heteroatoms, and are N and O; or wherein exactly 2 of said ring atoms are ring heteroatoms, and are N and S) which additionally, bear one or more substituents include the following:

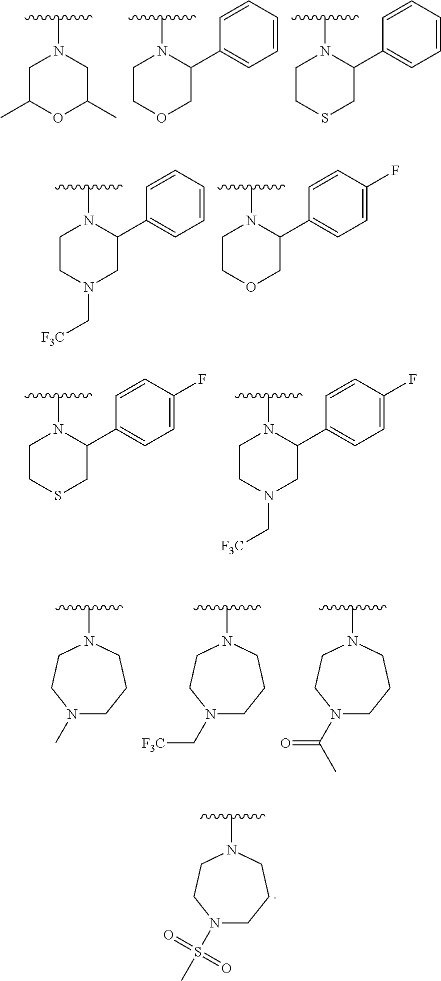

Examples of -J¹ groups which additionally bear at least one substituent that is phenyl include the following:

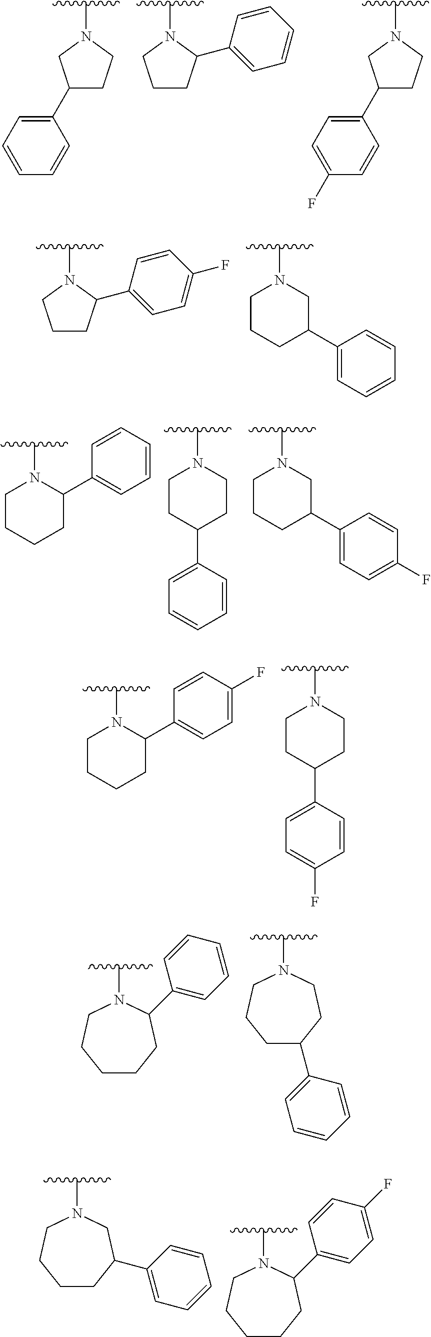

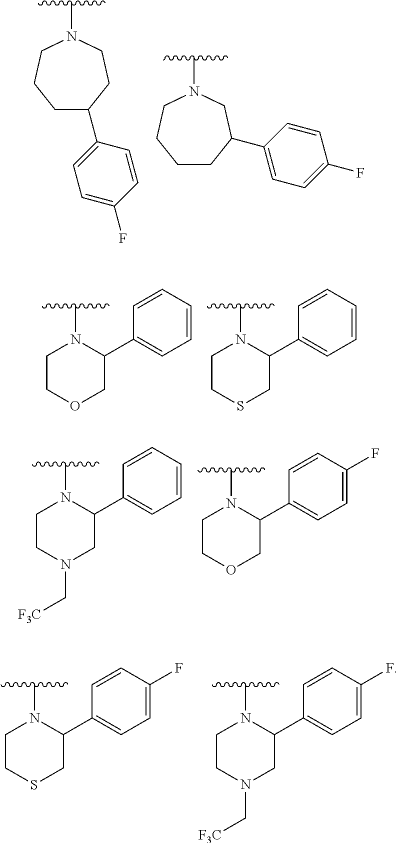

In one embodiment, -J¹, if present, is independently:

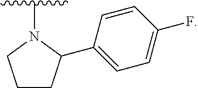

In one embodiment, -J¹, if present, is independently selected from the groups -J¹ shown in the compounds described under the heading “Examples of Specific Embodiments”.

### The Group -J²

In one embodiment, -J², if present, is independently a fused bicyclic non-aromatic heterocyclyl group having from 7 to 12 ring atoms, wherein exactly 1 of said ring atoms is a ring heteroatom, and is N, or exactly 2 of said ring atoms are ring heteroatoms, and are both N, or exactly 2 of said ring atoms are ring heteroatoms, and are N and O, or exactly 2 of said ring atoms are ring heteroatoms, and are N and S, or exactly 3 of said ring atoms are ring heteroatoms, one of which is N, and each of the other two is independently N, O, or S, and wherein said fused bicyclic non-aromatic heterocyclyl group is optionally substituted, for example, with one or more substituents as discussed below under the heading “Optional Substituents on -J¹ and -J²”, and wherein -J² is attached via one of said ring atoms which is N.

For the avoidance of doubt, when -J² is attached (i.e., to the carbonyl group shown in Formulae (I) and (II)) via one of said ring atoms which is N, the resulting compound is an amide.

For the avoidance of doubt, it is not intended that substituents on -J² form a ring fused to said fused bicyclic non-aromatic heterocyclyl group; that is, substituents on -J², if present, do not form a ring fused to said fused bicyclic non-aromatic heterocyclyl group. For example, it is not intended that -J² encompass benzoisoquinolinyl. However, it is intended that -J² may bear, for example, a substitutent that is or comprises a ring, for example, a substituent that is phenyl.

In one embodiment, exactly 1 of said -J² ring atoms is a ring heteroatom, and is N.

In one embodiment, exactly 2 of said -J² ring atoms are ring heteroatoms, and are both N.

In one embodiment, exactly 2 of said -J² ring atoms are ring heteroatoms, and are N and O.

In one embodiment, exactly 2 of said -J² ring atoms are ring heteroatoms, and are N and S.

In one embodiment, exactly 3 of said -J² ring atoms are ring heteroatoms, and are N, O, and O.

In one embodiment, exactly 3 of said -J² ring atoms are ring heteroatoms, and are N,N, and O.

In one embodiment, exactly 3 of said -J² ring atoms are ring heteroatoms, and are N,N, and S.

In one embodiment, exactly 3 of said -J² ring atoms are ring heteroatoms, and are N, O, and S.

In one embodiment, said -J² fused bicyclic non-aromatic heterocyclyl group has 9 to 10 ring atoms.

In one embodiment, said -J² fused bicyclic non-aromatic heterocyclyl group has 9 ring atoms.

In one embodiment, said -J² fused bicyclic non-aromatic heterocyclyl group has 10 ring atoms.

In one embodiment, -J², if present, is independently selected from the following groups and is optionally substituted, for example, with one or more substituents as discussed below under the heading “Optional Substituents on -J¹ and -J²”, for example, one or more substituents selected from saturated aliphatic C₁₋₄alkyl:

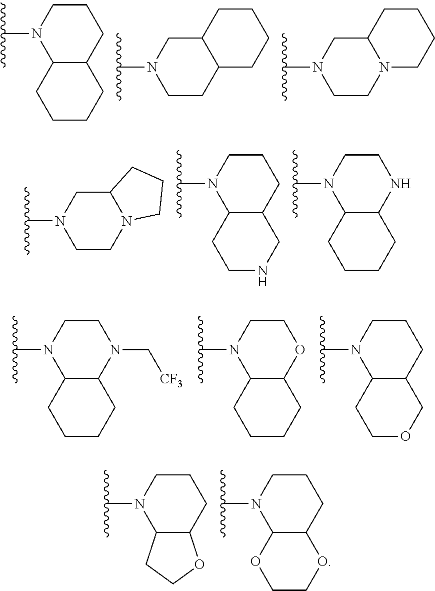

In one embodiment, -J², if present, is independently selected from the following groups and is optionally substituted, for example, with one or more substituents as discussed below under the heading “Optional Substituents on -J¹ and -J²”, for example, one or more substituents selected from saturated aliphatic C₁₋₄alkyl:

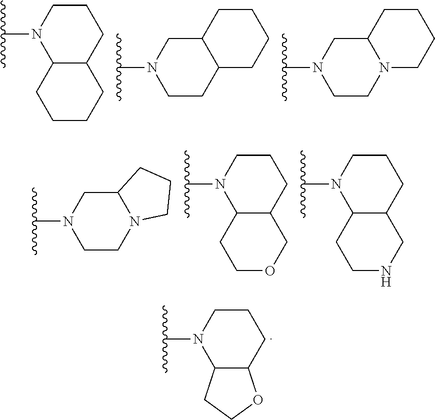

In one embodiment, -J², if present, is independently selected from the following groups and is optionally substituted, for example, with one or more substituents as discussed below under the heading “Optional Substituents on -J¹ and -J²”, for example, one or more substituents selected from saturated aliphatic C₁₋₄alkyl:

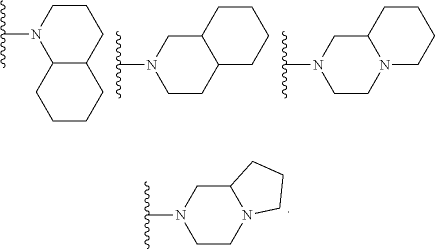

In one embodiment, -J², if present, is independently selected from the following groups and is optionally substituted, for example, with one or more substituents as discussed below under the heading “Optional Substituents on -J¹ and -J²”, for example, one or more substituents selected from saturated aliphatic C₁₋₄alkyl:

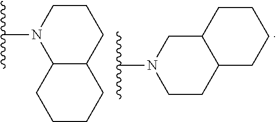

In one embodiment, -J², if present, is independently the following group and is optionally substituted, for example, with one or more substituents as discussed below under the heading “Optional Substituents on -J¹ and -J²”, for example, one or more substituents selected from saturated aliphatic C₁₋₄alkyl:

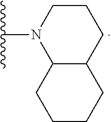

In one embodiment, -J², if present, is independently:

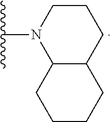

In one embodiment, -J², if present, is independently selected from:

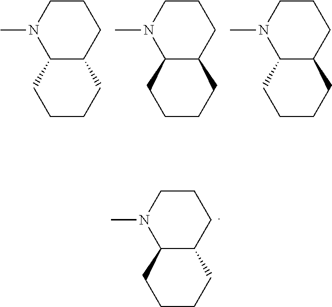

In one embodiment, -J², if present, is independently selected from:

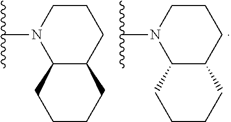

In one embodiment, -J², if present, is independently selected from the groups -J² shown in the compounds described under the heading “Examples of Specific Embodiments”.

### Optional Substituents on the Groups —R^{5A1}, —R^{5B1}, —R^{5A2}, and —R^{5B2}

In one embodiment, —R^{5A1}, if present, is independently optionally substituted.

In one embodiment, —R^{5A1}, if present, is independently unsubstituted.

In one embodiment, —R^{5B1}, if present, is independently optionally substituted.

In one embodiment, —R^{5B1}, if present, is independently unsubstituted.

In one embodiment, —R^{5A2}, if present, is independently optionally substituted.

In one embodiment, —R^{5A2}, if present, is independently unsubstituted.

In one embodiment, —R^{5B2}, if present, is independently optionally substituted.

In one embodiment, —R^{5B2}, if present, is independently unsubstituted.

In one embodiment, optional substituents on —R^{5A1}, if present, and optional substituents on —R^{5B1}, if present, and optional substituents on —R^{5A2}, if present, and optional substituents on —R^{5B2}, if present, are independently selected from:
—R^{Q}, —R^{R}, —R^{L}—R^{R},
—F, —Cl, —Br,
—CN,
—NO₂,
—CF₃, —OCF₃,
—SR^{P},
—OH, —OR^{P},
—R^{L}—OH, —R^{L}—OR^{P},
—O—R^{L}—OH, —O—R^{L}—OR^{P},
—NH—R^{L}—OH, —NH—R^{L}—OR^{P},
—NR^{Q}—R^{L}—OH, —NR^{Q}—R^{L}—OR^{P},
—NH₂, —NHR^{P}, —NR^{P}₂, —R^{M},
—R^{L}—NH₂, —R^{L}—NHR^{P}, —R^{L}—NR^{P}₂, —R^{L}—R^{M},
—O—R^{L}—NH₂, —O—R^{L}—NHR^{P}, —O—R^{L}—NR^{P}₂, —O—R^{L}—R^{M},
—NH—R^{L}—NH₂, —NH—R^{L}—NHR^{P}, —NH—R^{L}—NR^{P}₂, —NH—R^{L}—R^{M},
—NR^{Q}—R^{L}—NH₂, —NR^{Q}—R^{L}—NHR^{P}, —NR^{Q}—R^{L}—NR^{P}₂, —NR^{Q}—R^{L}—R^{M},
—S(═O)₂NH₂, —S(═O)₂NHR^{P}, —S(═O)₂NR^{P}₂, —S(═O)₂R^{M},
—R^{L}—S(═O)₂NH₂, —R^{L}—S(═O)₂NHR^{P}, —R^{L}—S(═O)₂NR^{P}₂, —R^{L}—S(═O)₂R^{M},
—O—R^{L}—S(═O)₂NH₂, —O—R^{L}—S(═O)₂NHR^{P}, —O—R^{L}—S(═O)₂NR^{P}₂, —O—R^{L}—S(═O)₂R^{M},
—NH—R^{L}—S(═O)₂NH₂, —NH—R^{L}—S(═O)₂NHR^{P},
—NH—R^{L}—S(═O)₂NR^{P}₂, —NH—R^{L}—S(═O)₂R^{M},
—NR^{Q}—R^{L}—S(═O)₂NH₂, —NR^{Q}—R^{L}—S(═O)₂NHR^{P},
—NR^{Q}—R^{L}—S(═O)₂NR^{P}₂, —NR^{Q}—R^{L}—S(═O)₂R^{M},
—NHS(═O)₂R^{P}, —NHS(═O)₂R^{M},
—NR^{Q}S(═O)₂R^{P}, —NR^{Q}S(═O)₂R^{M},
—R^{L}—NHS(═O)₂R^{P}, —R^{L}—NHS(═O)₂R^{M},
—R^{L}—NR^{Q}S(═O)₂R^{P}, —R^{L}—NR^{Q}S(═O)₂R^{M},
—O—R^{L}—NHS(═O)₂R^{P}, —O—R^{L}—NHS(═O)₂R^{M},
—O—R^{L}—NR^{Q}S(═O)₂R^{P}, —O—R^{L}—NR^{Q}S(═O)₂R^{M},
—NH—R^{L}—NHS(═O)₂R^{P}, —NH—R^{L}—NHS(═O)₂R^{M},
—NH—R^{L}—NR^{Q}S(═O)₂R^{P}, —NH—R^{L}—NR^{Q}S(═O)₂R^{M},
—NR^{Q}—R^{L}—NHS(═O)₂R^{P}, —NR^{Q}—R^{L}—NHS(═O)₂R^{M},
—NR^{Q}—R^{L}—NR^{Q}S(═O)₂R^{P}, —NR^{Q}—R^{L}—NR^{Q}S(═O)₂R^{M},
—S(═O)₂R^{P}, —S(═O)₂R^{M},
—R^{L}—S(═O)₂R^{P}, —R^{L}—S(═O)₂R^{M},
—O—R^{L}—S(═O)₂R^{P}, —O—R^{L}—S(═O)₂R^{M},
—NH—R^{L}—S(═O)₂R^{P}, —NH—R^{L}—S(═O)₂R^{M},
—NR^{Q}—R^{L}—S(═O)₂R^{P}, —NR^{Q}—R^{L}—S(═O)₂R^{M},
—C(═O)R^{P},
—C(═O)OH, —C(═O)OR^{P},
—R^{L}—C(═O)OH, —R^{L}—C(═O)OR^{P},
—O—R^{L}—C(═O)OH, —O—R^{L}—C(═O)OR^{P},
—NH—R^{L}—C(═O)OH, —NH—R^{L}—C(═O)OR^{P},
—NR^{Q}—R^{L}—C(═O)OH, —NR^{Q}—R^{L}—C(═O)OR^{P},
—C(═O)NH₂, —C(═O)NHR^{P}, —C(═O)NR^{P}₂, —C(═O)R^{M},
—R^{L}—C(═O)NH₂, —R^{L}—C(═O)NHR^{P}, —R^{L}—C(═O)NR^{P}₂, —R^{L}—C(═O)R^{M},
—O—R^{L}—C(═O)NH₂, —O—R^{L}—C(═O)NHR^{P}, —O—R^{L}—C(═O)NR^{P}₂, —O—R^{L}—C(═O)R^{M},
—NH—R^{L}—C(═O)NH₂, —NH—R^{L}—C(═O)NHR^{P},
—NH—R^{L}—C(═O)NR^{P}₂, —NH—R^{L}—C(═O)R^{M},
—NR^{Q}—R^{L}—C(═O)NH₂, —NR^{Q}—R^{L}—C(═O)NHR^{P},
—NR^{Q}— R^{L}—C(═O)NR^{P}₂, —NR^{Q}—R^{L}—C(═O)R^{M};
—NHC(═O)R^{P}, —NR^{Q}C(═O)R^{P},
—R^{L}—NHC(═O)R^{P}, —R^{L}—NR^{Q}C(═O)R^{P},
—O—R^{L}—NHC(═O)R^{P}, —O—R^{L}—NR^{Q}C(═O)R^{P},
—NH—R^{L}—NHC(═O)R^{P}, —NH—R^{L}—NR^{Q}C(═O)R^{P},
—NR^{Q}—R^{L}—NHC(═O)R^{P}, —NR^{Q}—R^{L}—NR^{Q}C(═O)R^{P},
—O—C(═O)NH₂, —O—C(═O)NHR^{P},
—O—C(═O)NR^{P}₂, —O—C(═O)R^{M},
—R^{L}—O—C(═O)NH₂, —R^{L}—O—C(═O)NHR^{P},
—R^{L}—O—C(═O)NR^{P}₂, —R^{L}—O—C(═O)R^{M},
—O—R^{L}—O—C(═O)NH₂, —O—R^{L}—O—C(═O)NHR^{P},
—O—R^{L}—O—C(═O)NR^{P}₂, —O—R^{L}—O—C(═O)R^{M},
—NH—R^{L}—O—C(═O)NH₂, —NH—R^{L}—O—C(═O)NHR^{P},
—NH—R^{L}—O—C(═O)NR^{P}₂, —NH—R^{L}—O—C(═O)R^{M},
—NR^{Q}—R^{L}—O—C(═O)NH₂, —NR^{Q}—R^{L}—O—C(═O)NHR^{P},
—NR^{Q}—R^{L}—O—C(═O)NR^{P}₂, —NR^{Q}—R^{L}—O—C(═O)R^{M},
—NH—C(═O)OR^{P}, —NR^{Q}—C(═O)OR^{P},
—R^{L}—NH—C(═O)OR^{P}, —R^{L}—NR^{Q}—C(═O)OR^{P},
—O—R^{L}—NH—C(═O)OR^{P}, —O—R^{L}—NR^{Q}—C(═O)OR^{P},
—NH—R^{L}—NH—C(═O)OR^{P}, —NH—R^{L}—NR^{Q}—C(═O)OR^{P},
—NR^{Q}—R^{L}—NH—C(═O)OR^{P}, —NR^{Q}—R^{L}—NR^{Q}—C(═O)OR^{P},
—NH—C(═O)NH₂, —NH—C(═O)NHR^{P},
—NH—C(═O)NR^{P}₂, —NH—C(═O)R^{M},
—NR^{Q}—C(═O)NH₂, —NR^{Q}—C(═O)NHR^{P},
—NR^{Q}—C(═O)NR^{P}₂, —NR^{Q}—C(═O)R^{M},
—R^{L}—NH—C(═O)NH₂, —R^{L}—NH—C(═O)NHR^{P},
—R^{L}—NH—C(═O)NR^{P}₂, —R^{L}—NH—C(═O)R^{M},
—R^{L}—NR^{Q}—C(═O)NH₂, —R^{L}—NR^{Q}—C(═O)NHR^{P},
—R^{L}—NR^{Q}—C(═O)NR^{P}₂, —R^{L}—NR^{Q}—C(═O)R^{M},
—O—R^{L}—NH—C(═O)NH₂, —O—R^{L}—NH—C(═O)NHR^{P},
—O—R^{L}—NH—C(═O)NR^{P}₂, —O—R^{L}—NH—C(═O)R^{M},
—O—R^{L}—NR^{Q}—C(═O)NH₂, —O—R^{L}—NR^{Q}—C(═O)NHR^{P},
—R^{L}—NR^{R}—C(═O)NR^{P}₂, —O—R^{L}—NR^{R}—C(═O)R^{M}, —NH—R^{L}—NH—C(═O)NH₂, —NH—R^{L}—NH—C(═O)NHR^{P},
—NH—R^{L}—NH—C(═O)NR^{P}₂, —NH—R^{L}—NH—C(═O)R^{M},
—NH—R^{L}—NR^{R}—C(═O)NH₂, —NH—R^{L}—NR^{R}—C(═O)NHR^{P},
—NH—R^{L}—NR^{R}—C(═O)NR^{P}₂, —NH—R^{L}—NR^{R}—C(═O)R^{M},
—NR^{R}—R^{L}—NH—C(═O)NH₂, —NR^{R}—R^{L}—NH—C(═O)NHR^{P},
—NR^{R}—R^{L}—NH—C(═O)NR^{P}₂, —NR^{R}—R^{L}—NH—C(═O)R^{M},
—NR^{R}—R^{L}—NR^{R}—C(═O)NH₂, —NR^{R}—R^{L}—NR^{R}—C(═O)NHR^{P},
—NR^{R}—R^{L}—NR^{R}—C(═O)NR^{P}₂, and —NR^{R}—R^{L}—NR^{R}—C(═O)R^{M},
═O;
or two adjacent substituents, if present, together form —O—CH₂—O— or —O—CH₂CH₂—O—;
or two adjacent substituents, if present, together form —O—C(═O)—NH—, —O—C(═O)—NR^{Q}—, —NR^{R}—C(═O)—NH—, —NH—C(═O)—NR^{R}—, or —NR^{R}—C(═O)—NR^{R}—.

In one embodiment, optional substituents on —R^{5M}, if present, and optional substituents on —R^{5B1}, if present, and optional substituents on —R^{5A2}, if present, and optional substituents on —R^{5B2}, if present, are independently selected from:
—R^{L}—R^{R},
—F, —Cl, —Br,
—CN,
—NO₂,
—CF₃, —OCF₃,
—OH, —OR^{P},
—R^{L}—OH, —R^{L}—OR^{P},
—O—R^{L}—OH, —O—R^{L}—OR^{P},
—NH—R^{L}—OH, —NH—R^{L}—OR^{P},
—NH₂, —NHR^{P}, —NR^{P}₂, —R^{M},
—R^{L}—NH₂, —R^{L}—NHR^{P}, —R^{L}—NR^{P}₂, —R^{L}—R^{M},
—O—R^{L}—NH₂, —O—R^{L}—NHR^{P}, —O—R^{L}—NR^{P}₂, —O—R^{L}—R^{M},
—NH—R^{L}—NH₂, —NH—R^{L}—NHR^{P}, —NH—R^{L}—NR^{P}₂, —NH—R^{L}—R^{M},
—S(═O)₂NH₂, —S(═O)₂NHR^{P}, —S(═O)₂NR^{P}₂, —S(═O)₂R^{M},
—R^{L}—S(═O)₂NH₂, —R^{L}—S(═O)₂NHR^{P}, —R^{L}—S(═O)₂NR^{P}₂, —R^{L}—S(═O)₂R^{M},
—O—R^{L}—S(═O)₂NH₂, —O—R^{L}—S(═O)₂NHR^{P}, —O—R^{L}—S(═O)₂NR^{P}₂, —O—R^{L}—S(═O)₂R^{M},
—NH—R^{L}—S(═O)₂NH₂, —NH—R^{L}—S(═O)₂NHR^{P},
—NH—R^{L}—S(═O)₂NR^{P}₂, —NH—R^{L}—S(═O)₂R^{M},
—NHS(═O)₂R^{P}, —NHS(═O)₂R^{M},
—NR^{Q}S(═O)₂R^{P}, —NR^{Q}S(═O)₂R^{M},
—R^{L}—NHS(═O)₂R^{P}, —R^{L}—NHS(═O)₂R^{M},
—R^{L}—NR^{Q}S(═O)₂R^{P}, —R^{L}—NR^{Q}S(═O)₂R^{M},
—O—R^{L}—NHS(═O)₂R^{P}, —O—R^{L}—NHS(═O)₂R^{M},
—O—R^{L}—NR^{Q}S(═O)₂R^{P}, —O—R^{L}—NR^{Q}S(═O)₂R^{M},
—NH—R^{L}—NHS(═O)₂R^{P}, —NH—R^{L}—NHS(═O)₂R^{M},
—NH—R^{L}—NR^{Q}S(═O)₂R^{P}, —NH—R^{L}—NR^{Q}S(═O)₂R^{M},
—S(═O)₂R^{P}, —S(═O)₂R^{M},
—R^{L}—S(═O)₂R^{P}, —R^{L}—S(═O)₂R^{M},
—O—R^{L}—S(═O)₂R^{P}, —O—R^{L}—S(═O)₂R^{M},
—NH—R^{L}—S(═O)₂R^{P}, —NH—R^{L}—S(═O)₂R^{M},
—C(═O)OH, —C(═O)OR^{P},
—R^{L}—C(═O)OH, —R^{L}—C(═O)OR^{P},
—O—R^{L}—C(═O)OH, —O—R^{L}—C(═O)OR^{P},
—NH—R^{L}—C(═O)OH, —NH—R^{L}—C(═O)OR^{P},
—C(═O)NH₂, —C(═O)NHR^{P}, —C(═O)NR^{P}₂, —C(═O)R^{M},
—R^{L}—C(═O)NH₂, —R^{L}—C(═O)NHR^{P}, —R^{L}—C(═O)NR^{P}₂, —R^{L}—C(═O)R^{M},
—O—R^{L}—C(═O)NH₂, —O—R^{L}—C(═O)NHR^{P}, —O—R^{L}—C(═O)NR^{P}₂, —O—R^{L}—C(═O)R^{M},
—NH—R^{L}—C(═O)NH₂, —NH—R^{L}—C(═O)NHR^{P},
—NH—R^{L}—C(═O)NR^{P}₂, —NH—R^{L}—C(═O)R^{M},
—NHC(═O)R^{P}, —NR^{Q}C(═O)R^{P},
—R^{L}—NHC(═O)R^{P}, —R^{L}—NR^{Q}C(═O)R^{P},
—O—R^{L}—NHC(═O)R^{P}, —O—R^{L}—NR^{Q}C(═O)R^{P},
—NH—R^{L}—NHC(═O)R^{P}, —NH—R^{L}—NR^{Q}C(═O)R^{P},
—O—C(═O)NH₂, —O—C(═O)NHR^{P},
—O—C(═O)NR^{P}₂, —O—C(═O)R^{M},
—R^{L}—O—C(═O)NH₂, —R^{L}—O—C(═O)NHR^{P},
—R^{L}—O—C(═O)NR^{P}₂, —R^{L}—O—C(═O)R^{M},
—O—R^{L}—O—C(═O)NH₂, —O—R^{L}—O—C(═O)NHR^{P},
—O—R^{L}—O—C(═O)NR^{P}₂, —O—R^{L}—O—C(═O)R^{M},
—NH—R^{L}—O—C(═O)NH₂, —NH—R^{L}—O—C(═O)NHR^{P},
—NH—R^{L}—O—C(═O)NR^{P}₂, —NH—R^{L}—O—C(═O)R^{M},
—NH—C(═O)OR^{P}, —NR^{R}—C(═O)OR^{P},
—R^{L}—NH—C(═O)OR^{P}, —R^{L}—NR^{Q}—C(═O)OR^{P},
—O—R^{L}—NH—C(═O)OR^{P}, —O—R^{L}—NR^{R}—C(═O)OR^{P},
—NH—R^{L}—NH—C(═O)OR^{P}, —NH—R^{L}—NR^{R}—C(═O)OR^{P},
—NH—C(═O)NH₂, —NH—C(═O)NHR^{P},
—NH—C(═O)NR^{P}₂, —NH—C(═O)R^{M},
—R^{L}—NH—C(═O)NH₂, —R^{L}—NH—C(═O)NHR^{P},
—R^{L}—NH—C(═O)NR^{P}₂, —R^{L}—NH—C(═O)R^{M},
—R^{L}—NR^{R}—C(═O)NH₂, —R^{L}—NR^{R}—C(═O)NHR^{P},
—R^{L}—NR^{R}—C(═O)NR^{P}₂, —R^{L}—NR^{R}—C(═O)R^{M},
—O—R^{L}—NH—C(═O)NH₂, —O—R^{L}—NH—C(═O)NHR^{P},
—O—R^{L}—NH—C(═O)NR^{P}₂, —O—R^{L}—NH—C(═O)R^{M},
—O—R^{L}—NR^{Q}—C(═O)NH₂, —O—R^{L}—NR^{R}—C(═O)NHR^{P},
—O—R^{L}—NR^{R}—C(═O)NR^{P}₂, —O—R^{L}—NR⁴—C(═O)R^{M},
—NH—R^{L}—NH—C(═O)NH₂, —NH—R^{L}—NH—C(═O)NHR^{P},
—NH—R^{L}—NH—C(═O)NR^{P}₂, —NH—R^{L}—NH—C(═O)R^{M},
—NH—R^{L}—NR^{R}—C(═O)NH₂, —NH—R^{L}—NR^{Q}—C(═O)NHR^{P},
—NH—R^{L}—NR⁴—C(═O)NR^{P}₂, and —NH—R^{L}—NR^{R}—C(═O)R^{M}, and
═O;
or two adjacent substituents, if present, together form —O—CH₂—O— or —O—CH₂CH₂—O—;
or two adjacent substituents, if present, together form —O—C(═O)—NH—, —O—C(═O)—NR⁴—, —NR^{R}—C(═O)—NH—, —NH—C(═O)—NR⁴—, or —NR⁴—C(═O)—NR^{Q}—.

In one embodiment, optional substituents on —R^{5A1} if present, and optional substituents on —R^{5B1}, if present, and optional substituents on —R^{5A2}, if present, and optional substituents on —R^{5B2}, if present, are independently selected from:
—R⁴, —R^{L}—R^{P},
—F, —Cl, —Br,
—CN,
—NO₂,
—CF₃, —OCF₃,
—OH, —OR^{P},
—R^{L}—OH, —R^{L}—OR^{P},
—NH₂, —NHR^{P}, —NR^{P}₂, —R^{M},
—R^{L}—NH₂, —R^{L}—NHR^{P}, —R^{L}—NR^{P}₂, —R^{L}—R^{M},
—S(═O)₂NH₂, —S(═O)₂NHR^{P}, —S(═O)₂NR^{P}₂, —S(═O)₂R^{M},
—R^{L}—S(═O)₂NH₂, —R^{L}—S(═O)₂NHR^{P}, —R^{L}—S(═O)₂NR^{P}₂, —R^{L}—S(═O)₂R^{M},
—NHS(═O)₂R^{P}, —NHS(═O)₂R^{M},
—NR^{Q}S(═O)₂R^{P}, —NR^{Q}S(═O)₂R^{M},
—R^{L}—NHS(═O)₂R^{P}, —R^{L}—NHS(═O)₂R^{M},
—R^{L}—NR^{Q}S(═O)₂R^{P}, —R^{L}—NR^{Q}S(═O)₂R^{M},
—S(═O)₂R^{P}, —S(═O)₂R^{M},
—R^{L}—S(═O)₂R^{P}, —R^{L}—S(═O)₂R^{M},
—C(═O)OH, —C(═O)OR^{P},
—R^{L}—C(═O)OH, —R^{L}—C(═O)OR^{P},
—C(═O)NH₂, —C(═O)NHR^{P}, —C(═O)NR^{P}₂, —C(═O)R^{M},
—R^{L}—C(═O)NH₂, —R^{L}—C(═O)NHR^{P}, —R^{L}—C(═O)NR^{P}₂, —R^{L}—C(═O)R^{M},
—NHC(═O)R^{P}, —NR^{Q}C(═O)R^{P},
—R^{L}—NHC(═O)R^{P}, —R^{L}—NR^{Q}C(═O)R^{P},
—O—C(═O)NH₂, —O—C(═O)NHR^{P},
—O—C(═O)NR^{P}₂, —O—C(═O)R^{M},
—R^{L}—O—C(═O)NH₂, —R^{L}—O—C(═O)NHR^{P},
—R^{L}—O—C(═O)NR^{P}₂, —R^{L}—O—C(═O)R^{M},
—R^{L}—NH—C(═O)OR^{P}, —R^{L}—NR^{Q}—C(═O)OR^{P},
—NH—C(═O)NH₂, —NH—C(═O)NHR^{P},
—NH—C(═O)NR^{P}₂, —NH—C(═O)R^{M},
—R^{L}—NH—C(═O)NH₂, —R^{L}—NH—C(═O)NHR^{P},
—R^{L}—NH—C(═O)NR^{P}₂, —R^{L}—NH—C(═O)R^{M},
—R^{L}—NR^{Q}—C(═O)NH₂, —R^{L}—NR^{Q}—C(═O)NHR^{P},
—R^{L}—NR^{R}—C(═O)NR^{P}₂, and —R^{L}—NR^{R}—C(═O)R^{M}, and
═O;
or two adjacent substituents, if present, together form —O—CH₂—O— or —O—CH₂CH₂—O—;
or two adjacent substituents, if present, together form —O—C(═O)—NH—, —O—C(═O)—NR^{R}—, —NR^{R}—C(═O)—NH—, —NH—C(═O)—NR^{R}—, or —NR^{R}—C(═O)—NR^{R}—.

In one embodiment, optional substituents on —R^{5M}, if present, and optional substituents on —R^{5B1}, if present, and optional substituents on —R^{5A2}, if present, and optional substituents on —R^{5B2}, if present, are independently selected from:
—R^{X1}, —CF₃,
—F, —Cl, —Br,
—OH, —OR^{X1}, —OCF₃,
—CN,
—NO₂,
—NH₂, —NHR^{X1}, —NR^{X1}₂, -M,
—R^{XL}—NH₂, —R^{XL}—NHR^{X1}, —R^{XL}—NR^{X1}₂, —R^{XL}-M,
—NHC(═O)R^{X1}, —NR^{X1}C(═O)R^{X1},
—R^{XL}—NHC(═O)R^{X1}, —R^{XL}—NR^{X1}C(═O)R^{X1},
—C(═O)OH, —C(═O)OR^{X1},
—R^{XL}—C(═O)OH, —R^{XL}—C(═O)OR^{X1},
—C(═O)NH₂, —C(═O)NHR^{X1}, —C(═O)NR^{X1}₂, —C(═O)M,
—R^{XL}—C(═O)NH₂, —R^{XL}—C(═O)NHR^{X1}, —R^{XL}—C(═O)NR^{X1}₂, —R^{XL}—C(═O)M,
—S(═O)₂NH₂, —S(═O)₂NHR^{X1}, —S(═O)₂NR^{X1}₂, —S(═O)₂M,
—NHS(═O)₂R^{X1}, —NR^{X1}S(═O)₂R^{X1},
—NHC(═O)NH₂, —NHC(═O)NHR^{X1}, —NHC(═O)NR^{X1}₂, —NHC(═O)M,
—NR^{X1}C(═O)NH₂, —NR^{X1}C(═O)NHR^{X1}, —NR^{X1}C(═O)NR^{X1}₂, —NR^{X1}C(═O)M, and
═O;
or two adjacent substituents, if present, together form —O—CH₂—O— or —O—CH₂CH₂—O—;
wherein:
each —R^{X1} is independently saturated aliphatic C₁₋₄alkyl or phenyl;
each is independently saturated aliphatic C₁₋₄alkylene; and
each -M is pyrrolidino, piperidino, piperazino, or morpholino, and is optionally substituted, for example, with one or more groups selected from saturated aliphatic C₁₋₄alkyl.

In one embodiment, optional substituents on —R^{5A1}, if present, and optional substituents on —R^{5B1}, if present, and optional substituents on —R^{5A2}, if present, and optional substituents on —R^{5B2}, if present, are independently selected from:
—R^{X1}, —F, —Cl, —Br, —CF₃, —OH, —OR^{X1}, —OCF₃, —CN, —NO₂, —NH₂, —NHR^{X1}, —NR^{X1}₂, —NHC(═O)R^{X1}, —NR^{X1}C(═O)R^{X1}, —C(═O)NH₂, —C(═O)NHR^{X1}, —C(═O)NR^{X1}₂, —S(═O)₂NH₂, —S(═O)₂NHR^{X1}, —S(═O)₂NR^{X1}₂, —NR^{X1}S(═O)₂R^{X1}, —NHC(═O)NH₂, —NHC(═O)NHR^{X1}, —NHC(═O)NR^{X1}₂, —NR^{X1}C(═O)NH₂, —NR^{X1}C(═O)NHR^{X1}, —NR^{X1}C(═O)NR^{X1}₂, and ═O;
wherein each —R^{X1} is independently saturated aliphatic C₁₋₄alkyl or phenyl.

In one embodiment, optional substituents on —R^{5A1}, if present, and optional substituents on —R^{5B1}, if present, and optional substituents on —R^{5A2}, if present, and optional substituents on —R^{5B2}, if present, are independently selected from:
—R^{X1}, —F, —Cl, —Br, —OH, —OR^{X1}, —NH₂, —NHR^{X1}, —NR^{X1}₂, —NHC(═O)R^{X1}, —NR^{X1}C(═O)R^{X1}, and ═O;
wherein each —R^{X1} is independently saturated aliphatic C₁₋₄alkyl or phenyl.

In one embodiment, optional substituents on —R^{5A1}, if present, and optional substituents on —R^{5B1}, if present, and optional substituents on —R^{5A2}, if present, and optional substituents on —R^{5B2}, if present, are independently selected from the substitutents on —R^{5A1}, —R^{5B1}, —R^{5A2}, and —R^{5B2} shown in the compounds described under the heading “Examples of Specific Embodiments”.

In one embodiment, optional substituents on —R^{5A1}, if present, are independently selected from the substitutents on —R^{5A1} shown in the compounds described under the heading “Examples of Specific Embodiments”.

In one embodiment, optional substituents on —R^{5B1}, if present, are independently selected from the substitutents on —R^{5B1} shown in the compounds described under the heading “Examples of Specific Embodiments”.

In one embodiment, optional substituents on —R^{5A2}, if present, are independently selected from the substitutents on —R^{5A2} shown in the compounds described under the heading “Examples of Specific Embodiments”.

In one embodiment, optional substituents on —R^{5B2}, if present, are independently selected from the substitutents on —R^{5B2} shown in the compounds described under the heading “Examples of Specific Embodiments”.

### Optional Substituents on -J¹ and -J²

In one embodiment, each of -J¹ and -J² is independently optionally substituted.

In one embodiment, each of -J¹ and -J² is independently unsubstituted.

In one embodiment, -J¹ is independently unsubstituted.

In one embodiment, -J² is independently unsubstituted.

Again, for the avoidance of doubt, it is not intended that substituents on -J¹ form a ring fused to said monocyclic non-aromatic heterocyclyl group; that is, substituents on -J¹, if present, do not form a ring fused to said monocyclic non-aromatic heterocyclyl group. For example, it is not intended that -J¹ encompass benzazepinyl. However, it is intended that -J¹ may bear, for example, a substitutent that is or comprises a ring, for example, a substituent that is phenyl.

Again, for the avoidance of doubt, it is not intended that substituents on -J² form a ring fused to said fused bicyclic non-aromatic heterocyclyl group; that is, substituents on -J², if present, do not form a ring fused to said fused bicyclic non-aromatic heterocyclyl group. For example, it is not intended that -J² encompass benzoisoquinolinyl. However, it is intended that -J² may bear, for example, a substitutent that is or comprises a ring, for example, a substituent that is phenyl.

In one embodiment, optional substituents on -J¹, if present, and optional substituents on -J², if present, are independently selected from:
substituents on carbon, independently selected from:
—R^{Q}, —R^{R}, —R^{L}—R^{R},
—F, —Cl, —Br,
—OH, —R^{L}—OH,
—OR^{P}, —R^{L}—OR^{P}, —O—R^{L}—OR^{P},
—SR^{P},
—NH₂, —NHR^{P}, —NR^{P}₂, —R^{M},
—NHC(═O)R^{P}, —NR^{Q}C(═O)R^{P},
—NH—C(═O)NH₂, —NH—C(═O)NHR^{P}, —NH—C(═O)NR^{P}₂, —NH—C(═O)R^{M},
—NR^{Q}—C(═O)NH₂, —NR^{Q}—C(═O)NHR^{P}, —NR^{Q}—C(═O)NR^{P}₂, —NR^{Q}—C(═O)R^{M},
—C(═O)NH₂, —C(═O)NHR^{P}, —C(═O)NR^{P}₂, —C(═O)R^{M},
—C(═O)OH, —C(═O)OR^{P},
—S(═O)₂NH₂, —S(═O)₂NHR^{P}, —S(═O)₂NR₂^{P}, —S(═O)₂R^{M},
—NHS(═O)₂R^{P}, —NR^{Q}S(═O)₂R^{P}, —NHS(═O)₂R^{M}, —NR^{Q}S(═O)₂R^{M},
—CN,
—R^{L}—S(═O)₂NH₂, —R^{L}—S(═O)₂NHR^{P}, —R^{L}—S(═O)₂NR^{P}₂, —R^{L}—S(═O)₂R^{M},
—R^{L}—NH₂, —R^{L}—NHR^{P}, —R^{L}—NR^{P}₂, —R^{L}—R^{M},
—R^{L}—NHC(═O)R^{P}, —R^{L}—NR^{Q}C(═O)R^{P},
—R^{L}—NHS(═O)₂R^{P}, —R^{L}—NR^{Q}S(═O)₂R^{P},
—R^{L}—NHS(═O)₂R^{M}, —R^{L}—NR^{Q}S(═O)₂R^{M},
—R^{L}—C(═O)OH, —R^{L}—C(═O)OR^{P},
—R^{L}—C(═O)NH₂, —R^{L}—C(═O)NHR^{P}, —R^{L}—C(═O)NR^{P}₂, and —R^{L}—C(═O)R^{M},
—R^{L}—NH—C(═O)NH₂, —R^{L}—NH—C(═O)NHR^{P}, —R^{L}—NH—C(═O)NR^{P}₂,
—R^{L}—NR^{Q}—C(═O)NH₂, —R^{L}—NR^{Q}—C(═O)NHR^{P}, —R^{L}—NR^{Q}—C(═O)NR^{P}₂; and
substituents on nitrogen, if present, independently selected from:
—R^{Q}, —R^{R}, —R^{L}—R^{R},
—C(═O)OR^{P},
—C(═O)R^{P},
—C(═O)—R^{L}—OH, —C(═O)—R^{L}—OR^{P},
—C(═O)NH₂, —C(═O)NHR^{P}, —C(═O)NR^{P}₂, —C(═O)R^{M},
—C(═O)—R^{L}—NH₂, —C(═O)—R^{L}—NHR^{P}, —C(═O)—R^{L}—NR^{P}₂, —C(═O)—R^{L}—R^{M},
—C(═O)—R^{L}—NHS(═O)₂R^{P}, —C(═O)—R^{L}—NR^{Q}S(═O)₂R^{P},
—C(═O)—R^{L}—NHS(═O)₂R^{M}, —C(═O)—R^{L}—NR^{Q}S(═O)₂R^{M},
—C(═O)—R^{L}—S(═O)₂NH₂, —C(═O)—R^{L}—S(═O)₂NHR^{P},
—C(═O)—R^{L}—S(═O)₂NR^{P}₂, —C(═O)—R^{L}—S(═O)₂R^{M},
—S(═O)₂NH₂, —S(═O)₂NHR^{P}, —S(═O)₂NR^{P}₂, —S(═O)₂R^{M},
—S(═O)₂R^{P},
—R^{L}—OH, —R^{L}—OR^{P},
—R^{L}—NH₂, —R^{L}—NHR^{P}, —R^{L}—NR^{P}₂, —R^{L}—R^{M},
—R^{L}—NHS(═O)₂R^{P}, —R^{L}—NR^{Q}S(═O)₂R^{P},
—R^{L}—S(═O)₂NH₂, —R^{L}—S(═O)₂NHR^{P}, —R^{L}—S(═O)₂NR^{P}₂, —R^{L}—S(═O)₂R^{M}, and
—R^{L}—S(═O)₂R^{P}.

In one embodiment, for the optional substituents on -J¹, if present, substituents on carbon, if present, are independently selected from:
—R^{Q}, —R^{R}, —R^{L}—R^{R},
—F,
—OH, —R^{L}—OH,
—OR^{P}, —R^{L}—OR^{P},
—NHC(═O)R^{P}, —NR^{Q}C(═O)R^{P},
—C(═O)NH₂, —C(═O)NHR^{P}, —C(═O)NR^{P}₂, —C(═O)R^{M},
—CN,
—R^{L}—S(═O)₂NH₂, —R^{L}—S(═O)₂NHR^{P}, —R^{L}—S(═O)₂NR^{P}₂, —R^{L}—S(═O)₂R^{M},
—R^{L}—NHC(═O)R^{P}, —R^{L}—NR^{Q}C(═O)R^{P},
—R^{L}—NHS(═O)₂R^{P}, —R^{L}—NR^{Q}S(═O)₂R^{P}, —R^{L}—NHS(═O)₂R^{M}, —R^{L}—NRQS(═O)₂R^{M},
—R^{L}—C(═O)OH, —R^{L}—C(═O)OR^{P},
—R^{L}—C(═O)NH₂, —R^{L}—C(═O)NHR^{P}, —R^{L}—C(═O)NR^{P}₂, and —R^{L}—C(═O)R^{M}.

In one embodiment, optional substituents on -J¹, if present, and optional substituents on -J², if present, are independently selected from:
substituents on carbon, independently selected from —F, —OH, —OR^{X2}, —R^{X2}, —CH₂C(═O)OR^{X2}, —CF₃, —CN, phenyl, benzyl, thienyl, and pyridyl; and
substituents on nitrogen, if present, independently selected from —R^{X2}, —CH₂CF₃, —S(═O)₂R^{X2} and —C(═O)R^{X2};
wherein each —R^{X2} is independently saturated aliphatic C₁₋₄alkyl; and
wherein each phenyl, benzyl, thienyl, and pyridyl is optionally substituted with one or more groups selected from: —F, —Cl, —R^{X22}, —OH, —OR^{X22}, —CN, —NH₂, —NHR^{X22}, —NR^{X22}₂; wherein each —R^{X22} is independently saturated aliphatic C₁₋₄alkyl.

In one embodiment, optional substituents on -J¹, if present, and optional substituents on -J², if present, are independently selected from:
substituents on carbon, independently selected from phenyl, benzyl, thienyl, and pyridyl; and
substituents on nitrogen, if present, independently selected from —R^{X2}, —CH₂CF₃, —S(═O)₂R^{X2} and —C(═O)R^{X2};
wherein each —R^{X2} is independently saturated aliphatic C₁₋₄alkyl; and
wherein each phenyl, benzyl, thienyl, and pyridyl is optionally substituted with one or more groups selected from: —F, —Cl, —R^{X22}, —OH, —OR^{X22}; —CN, —NH₂, —NHR^{X22}, —NR^{X22}₂; wherein each —R^{X22} is independently saturated aliphatic C₁₋₄alkyl.

In one embodiment, optional substituents on -J¹, if present, are independently selected from:
substituents on carbon, independently selected from phenyl; and
substituents on nitrogen, if present, independently selected from —R^{X2};
wherein each phenyl is optionally substituted with one or more groups selected from: —F, —Cl, —R^{X22}, —OH, —OR^{X22}; —CN, —NH₂, —NHR^{X22}, —NR^{X22}₂; wherein each —R^{X22} is independently saturated aliphatic C₁₋₄alkyl; and
wherein each —R^{X2} is independently saturated aliphatic C₁₋₄alkyl.

In one embodiment, optional substituents on -J¹, if present, and optional substituents on -J², if present, are independently selected from:
substituents on carbon, independently selected from —F, —OH, —OR^{X2}, —R^{X2}, —CF₃, —CN, phenyl, and pyridyl; and
substituents on nitrogen, if present, independently selected from —R^{X2}, —S(═O)₂R^{X2} and —C(═O)R^{X2};
wherein each —R^{X2} is independently saturated aliphatic C₁₋₄alkyl.

In one embodiment, optional substituents on -J¹, if present, and optional substituents on -J², if present, are independently selected from:
substituents on carbon, independently selected from —F, —OH, —OR^{X2}, and —R^{X2}; and
substituents on nitrogen, if present, independently selected from —R^{X2}, —S(═O)₂R^{X2} and —C(═O)R^{X2};
wherein each —R^{X2} is independently saturated aliphatic C₁₋₄alkyl.

In one embodiment, optional substituents on -J¹, if present, and optional substituents on -J², if present, are independently selected from:
substituents on carbon, independently selected from —F and —R^{X2}; and
substituents on nitrogen, if present, independently selected from —R^{X2};
wherein each —R^{X2} is independently saturated aliphatic C₁₋₄alkyl.

In one embodiment, optional substituents on -J¹, if present, and optional substituents on -J², if present, are independently selected from: saturated aliphatic C₁₋₄alkyl.

In one embodiment, optional substituents on -J¹, if present, and optional substituents on -J², if present, are independently selected from the substitutents on -J¹ and -J² shown in the compounds described under the heading “Examples of Specific Embodiments”.

In one embodiment, optional substituents on -J¹, if present, and optional substituents on -J², if present, are independently selected from the substitutents on -J¹ shown in the compounds described under the heading “Examples of Specific Embodiments”.

### The Group —R^{P}

In one embodiment, each —R^{P}, if present, is independently —R^{Q}, —R^{R}, or —R^{L}—R^{R}.

In one embodiment, each —R^{P}, if present, is independently —R^{Q}.

In one embodiment, each —R^{P}, if present, is independently —R^{R} or —R^{L}—R^{R}.

In one embodiment, each —R^{P}, if present, is independently —R^{R}.

### The Group —R^{Q}

In one embodiment, each —R^{Q}, if present, is independently saturated aliphatic C₁₋₄alkyl, and is optionally substituted, for example, with one or more fluorine atoms.

In one embodiment, each —R^{Q}, if present, is independently saturated aliphatic C₁₋₄alkyl.

### The Group —R^{R}

In one embodiment, each —R^{R}, if present, is independently phenyl or C₅₋₆heteroaryl (e.g., furanyl, thienyl, pyrrolyl, imidazolyl, pyrazolyl, triazolyl, tetrazolyl, oxazolyl, isoxazolyl, thiazolyl, isothiazolyl, pyridyl, pyrimidinyl, or pyridazinyl), and is optionally substituted, for example, with one or more substitutents independently selected from:
—F, —Cl, —Br,
—CF₃,
—OH, —OR^{K1}, —OCF₃,
—NH₂, —NHR^{K1}, —NR^{K1}₂, piperidino, piperazino, morpholino,
—NHC(═O)R^{K1}, —NR^{K1}C(═O)R^{K1},
—C(═O)OH, —C(═O)OR^{K1},
—C(═O)NH₂, —C(═O)NHR^{K1}, —C(═O)NR^{K1}₂,
—C(═O)-piperidino, —C(═O)-piperazino, —C(═O)-morpholino,
—NO₂, and
—CN;
wherein each —R^{K1} is independently saturated aliphatic C₁₋₄alkyl.

### The Group —R^{L}—

In one embodiment, each —R^{L}—, if present, is independently saturated aliphatic C₁₋₄alkylene.

In one embodiment, each —R^{L}—, if present, is independently saturated aliphatic

In one embodiment, each —R^{L}—, if present, is independently —CH₂—, —CH₂CH₂—, —CH₂CH₂CH₂— or —CH₂CH₂CH₂CH₂—.

In one embodiment, each —R^{L}—, if present, is independently —CH₂— or —CH₂CH₂—.

In one embodiment, each —R^{L}—, if present, is independently —CH₂—.

### The Group —R^{M}

In one embodiment, each —R^{M}, if present, is independently azetidino, pyrrolidino, piperidino, piperazino, morpholino, azepino, or diazepino, and is optionally substituted, for example,

on carbon, with one or more substitutents independently selected from:
—F, —R^{K2}, —OH, —OR^{K2}, —OCF₃, and —CN; and
   on nitrogen, if present, with one or more substitutents independently selected from:
—C(═O)Ph, —S(═O)₂R^{K2}, —S(═O)₂Ph, —S(═O)₂NH₂,
—S(═O)₂NHR^{K2}, —S(═O)₂NR^{K2}₂, and —S(═O)₂NHPh;
   wherein each —R^{K2} is independently saturated aliphatic C₁₋₄alkyl.

### Molecular Weight

In one embodiment, the AITZ compound has a molecular weight of from 208 to 1200.

In one embodiment, the bottom of range is from 220, 230, 240, 250, 275, 300, or 350.

In one embodiment, the top of range is 1100, 1000, 900, 800, 700, or 600.

In one embodiment, the range is 240 to 600.

### Combinations

It is appreciated that certain features of the invention, which are, for clarity, described in the context of separate embodiments, may also be provided in combination in a single embodiment. Conversely, various features of the invention, which are, for brevity, described in the context of a single embodiment, may also be provided separately or in any suitable sub-combination. All combinations of the embodiments pertaining to the chemical groups represented by the variables (e.g., —R³, —R^{3A}, —R^{3B}, —R⁴, —R^{4A}, —R^{4B}, —R⁵, —R^{5A1}, —R^{5A2}, —R^{5B1}, —R^{5B2}, —Z, -J¹, -J², —R^{P}, —R^{Q}, —R^{R}, —R^{L}, -L-, —R^{X1}, —R^{XL}, -M, —R^{X2}, —R^{X22}, —R^{K1}, —R^{K2}, etc.) are specifically embraced by the present invention and are disclosed herein just as if each and every combination was individually and explicitly disclosed, to the extent that such combinations embrace compounds that are stable compounds (i.e., compounds that can be isolated, characterised, and tested for biological activity). In addition, all sub-combinations of the chemical groups listed in the embodiments describing such variables are also specifically embraced by the present invention and are disclosed herein just as if each and every such sub-combination of chemical groups was individually and explicitly disclosed herein.

### Examples of Specific Embodiments

In one embodiment, the compounds are selected from compounds of the following formulae and pharmaceutically acceptable salts, hydrates, and solvates thereof:

| Code No. | Synthesis | Structure |
|---|---|---|
| AA-01 | 23 | 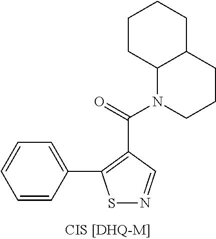 |
| AA-02 | 24 | 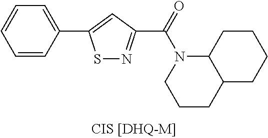 |

In one embodiment, the compounds are selected from compounds of the following formulae and pharmaceutically acceptable salts, hydrates, and solvates thereof:

| Code No. | Synthesis | Structure |
|---|---|---|
| BB-01 | 7 | 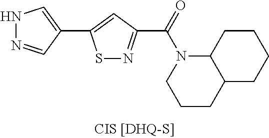 |
| BB-02 | 13 | 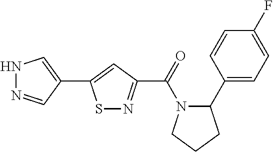 |
| BB-03 | 4 | 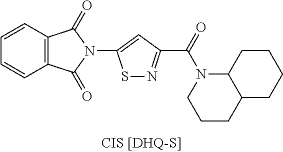 |

In one embodiment, the compounds are selected from compounds of the following formulae and pharmaceutically acceptable salts, hydrates, and solvates thereof:

| Code | | |
|---|---|---|
| No. | Synthesis | Structure |
| CC-01 | 16 | 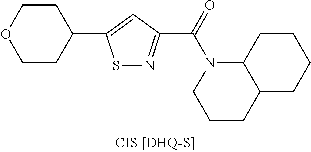 |
| CC-02 | 12 | 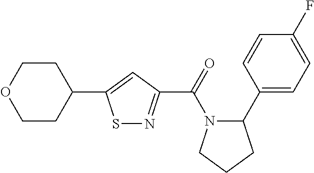 |

### Substantially Purified Forms

One aspect of the present invention pertains to AITZ compounds, as described herein, in substantially purified form and/or in a form substantially free from contaminants.

In one embodiment, the compound is in substantially purified form and/or in a form substantially free from contaminants.

In one embodiment, the compound is in a substantially purified form with a purity of least 50% by weight, e.g., at least 60% by weight, e.g., at least 70% by weight, e.g., at least 80% by weight, e.g., at least 90% by weight, e.g., at least 95% by weight, e.g., at least 97% by weight, e.g., at least 98% by weight, e.g., at least 99% by weight.

Unless specified, the substantially purified form refers to the compound in any stereoisomeric or enantiomeric form. For example, in one embodiment, the substantially purified form refers to a mixture of stereoisomers, i.e., purified with respect to other compounds. In one embodiment, the substantially purified form refers to one stereoisomer, e.g., optically pure stereoisomer. In one embodiment, the substantially purified form refers to a mixture of enantiomers. In one embodiment, the substantially purified form refers to an equimolar mixture of enantiomers (i.e., a racemic mixture, a racemate). In one embodiment, the substantially purified form refers to one enantiomer, e.g., optically pure enantiomer.

In one embodiment, the compound is in a form substantially free from contaminants wherein the contaminants represent no more than 50% by weight, e.g., no more than 40% by weight, e.g., no more than 30% by weight, e.g., no more than 20% by weight, e.g., no more than 10% by weight, e.g., no more than 5% by weight, e.g., no more than 3% by weight, e.g., no more than 2% by weight, e.g., no more than 1% by weight.

Unless specified, the contaminants refer to other compounds, that is, other than stereoisomers or enantiomers. In one embodiment, the contaminants refer to other compounds and other stereoisomers. In one embodiment, the contaminants refer to other compounds and the other enantiomer.

In one embodiment, the compound is in a substantially purified form with an optical purity of at least 60% (i.e., 60% of the compound, on a molar basis, is the desired stereoisomer or enantiomer, and 40% is undesired stereoisomer(s) or enantiomer), e.g., at least 70%, e.g., at least 80%, e.g., at least 90%, e.g., at least 95%, e.g., at least 97%, e.g., at least 98%, e.g., at least 99%.

### Isomers

Certain compounds may exist in one or more particular geometric, optical, enantiomeric, diasteriomeric, epimeric, atropic, stereoisomeric, tautomeric, conformational, or anomeric forms, including but not limited to, cis- and trans-forms; E- and Z-forms; c-, t-, and r-forms; endo- and exo-forms; R—, S—, and meso-forms; D- and L-forms; d- and l-forms; (+) and (−) forms; keto-, enol-, and enolate-forms; syn- and anti-forms; synclinal- and anticlinal-forms; α- and β-forms; axial and equatorial forms; boat-, chair-, twist-, envelope-, and halfchair-forms; and combinations thereof, hereinafter collectively referred to as “isomers” (or “isomeric forms”).

Note that, except as discussed below for tautomeric forms, specifically excluded from the term “isomers,” as used herein, are structural (or constitutional) isomers (i.e., isomers which differ in the connections between atoms rather than merely by the position of atoms in space). For example, a reference to a methoxy group, —OCH₃, is not to be construed as a reference to its structural isomer, a hydroxymethyl group, —CH₂OH. Similarly, a reference to ortho-chlorophenyl is not to be construed as a reference to its structural isomer, meta-chlorophenyl. However, a reference to a class of structures may well include structurally isomeric forms falling within that class (e.g., C₁₋₇alkyl includes n-propyl and iso-propyl; butyl includes n-, iso-, sec-, and tert-butyl; methoxyphenyl includes ortho-, meta-, and para-methoxyphenyl).

The above exclusion does not pertain to tautomeric forms, for example, keto-, enol-, and enolate-forms, as in, for example, the following tautomeric pairs: keto/enol (illustrated below), imine/enamine, amide/imino alcohol, amidine/amidine, nitroso/oxime, thioketone/enethiol, N-nitroso/hydroxyazo, and nitro/aci-nitro.

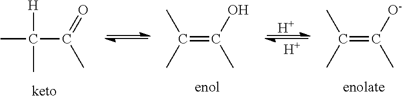

Note that specifically included in the term “isomer” are compounds with one or more isotopic substitutions. For example, H may be in any isotopic form, including ¹H, ²H (D), and ³H (T); C may be in any isotopic form, including ¹²C, ¹³C, and ¹⁴C; O may be in any isotopic form, including ¹⁶O and ¹⁸O; and the like.

Unless otherwise specified, a reference to a particular compound includes all such isomeric forms, including mixtures (e.g., racemic mixtures) thereof. Methods for the preparation (e.g., asymmetric synthesis) and separation (e.g., fractional crystallisation and chromatographic means) of such isomeric forms are either known in the art or are readily obtained by adapting the methods taught herein, or known methods, in a known manner.

### Salts

It may be convenient or desirable to prepare, purify, and/or handle a corresponding salt of the compound, for example, a pharmaceutically-acceptable salt. Examples of pharmaceutically acceptable salts are discussed in Berge et al., 1977, “Pharmaceutically Acceptable Salts,” J. Pharm. Sci., Vol. 66, pp. 1-19.

For example, if the compound is anionic, or has a functional group which may be anionic (e.g., —COOH may be —COO), then a salt may be formed with a suitable cation. Examples of suitable inorganic cations include, but are not limited to, alkali metal ions such as Na⁺ and K⁺, alkaline earth cations such as Ca²⁺ and Mg²⁺, and other cations such as Al⁺³. Examples of suitable organic cations include, but are not limited to, ammonium ion (i.e., NH₄⁺) and substituted ammonium ions (e.g., NH₃R⁺, NH₂R₂⁺, NHR₃⁺, NR₄⁺). Examples of some suitable substituted ammonium ions are those derived from: ethylamine, diethylamine, dicyclohexylamine, triethylamine, butylamine, ethylenediamine, ethanolamine, diethanolamine, piperazine, benzylamine, phenylbenzylamine, choline, meglumine, and tromethamine, as well as amino acids, such as lysine and arginine. An example of a common quaternary ammonium ion is N(CH₃)₄⁺.

If the compound is cationic, or has a functional group which may be cationic (e.g., —NH₂ may be —NH₃⁺), then a salt may be formed with a suitable anion. Examples of suitable inorganic anions include, but are not limited to, those derived from the following inorganic acids: hydrochloric, hydrobromic, hydroiodic, sulfuric, sulfurous, nitric, nitrous, phosphoric, and phosphorous.

Examples of suitable organic anions include, but are not limited to, those derived from the following organic acids: 2-acetyoxybenzoic, acetic, ascorbic, aspartic, benzoic, camphorsulfonic, cinnamic, citric, edetic, ethanedisulfonic, ethanesulfonic, fumaric, glucheptonic, gluconic, glutamic, glycolic, hydroxymaleic, hydroxynaphthalene carboxylic, isethionic, lactic, lactobionic, lauric, maleic, malic, methanesulfonic, mucic, oleic, oxalic, palmitic, pamoic, pantothenic, phenylacetic, phenylsulfonic, propionic, pyruvic, salicylic, stearic, succinic, sulfanilic, tartaric, toluenesulfonic, and valeric. Examples of suitable polymeric organic anions include, but are not limited to, those derived from the following polymeric acids: tannic acid, carboxymethyl cellulose.

Unless otherwise specified, a reference to a particular compound also includes salt forms thereof.

### Hydrates and Solvates

It may be convenient or desirable to prepare, purify, and/or handle a corresponding solvate of the compound. The term “solvate” is used herein in the conventional sense to refer to a complex of solute (e.g., compound, salt of compound) and solvent. If the solvent is water, the solvate may be conveniently referred to as a hydrate, for example, a mono-hydrate, a di-hydrate, a tri-hydrate, etc.

Unless otherwise specified, a reference to a particular compound also includes solvate and hydrate forms thereof.

### Chemically Protected Forms

It may be convenient or desirable to prepare, purify, and/or handle the compound in a chemically protected form. The term “chemically protected form” is used herein in the conventional chemical sense and pertains to a compound in which one or more reactive functional groups are protected from undesirable chemical reactions under specified conditions (e.g., pH, temperature, radiation, solvent, and the like). In practice, well known chemical methods are employed to reversibly render unreactive a functional group, which otherwise would be reactive, under specified conditions. In a chemically protected form, one or more reactive functional groups are in the form of a protected or protecting group (also known as a masked or masking group or a blocked or blocking group). By protecting a reactive functional group, reactions involving other unprotected reactive functional groups can be performed, without affecting the protected group; the protecting group may be removed, usually in a subsequent step, without substantially affecting the remainder of the molecule. See, for example, Protective Groups in Organic Synthesis (T. Green and P. Wuts; 4th Edition; John Wiley and Sons, 2006).

A wide variety of such “protecting,” “blocking,” or “masking” methods are widely used and well known in organic synthesis. For example, a compound which has two nonequivalent reactive functional groups, both of which would be reactive under specified conditions, may be derivatized to render one of the functional groups “protected,” and therefore unreactive, under the specified conditions; so protected, the compound may be used as a reactant which has effectively only one reactive functional group. After the desired reaction (involving the other functional group) is complete, the protected group may be “deprotected” to return it to its original functionality.

For example, a hydroxy group may be protected as an ether (—OR) or an ester (—OC(═O)R), for example, as: a t-butyl ether; a benzyl, benzhydryl(diphenylmethyl), or trityl(triphenylmethyl)ether; a trimethylsilyl or t-butyldimethylsilyl ether; or an acetyl ester (—OC(═O)CH₃, —OAc).

For example, an aldehyde or ketone group may be protected as an acetal (R—CH(OR)₂) or ketal (R₂C(OR)₂), respectively, in which the carbonyl group (>C═O) is converted to a diether (>C(OR)₂), by reaction with, for example, a primary alcohol. The aldehyde or ketone group is readily regenerated by hydrolysis using a large excess of water in the presence of acid.

For example, an amine group may be protected, for example, as an amide (—NRCO—R) or a urethane (—NRCO—OR), for example, as: a methyl amide (—NHCO—CH₃); a benzyloxy amide (—NHCO—OCH₂C₆H₅, —NH-Cbz); as a t-butoxy amide (—NHCO—OC(CH₃)₃, —NH-Boc); a 2-biphenyl-2-propoxy amide (—NHCO—OC(CH₃)₂C₆H₄C₆H₅, —NH-Bpoc), as a 9-fluorenylmethoxy amide (—NH-Fmoc), as a 6-nitroveratryloxy amide (—NH-Nvoc), as a 2-trimethylsilylethyloxy amide (—NH-Teoc), as a 2,2,2-trichloroethyloxy amide (—NH-Troc), as an allyloxy amide (—NH-Alloc), as a 2(-phenylsulfonyl)ethyloxy amide (—NH-Psec); or, in suitable cases (e.g., cyclic amines), as a nitroxide radical (>N—O.).

For example, a carboxylic acid group may be protected as an ester for example, as: an C₁₋₇alkyl ester (e.g., a methyl ester; a t-butyl ester); a C₁₋₇haloalkyl ester (e.g., a C₁₋₇-trihaloalkyl ester); a triC₁₋₇alkylsilyl-C₁₋₇alkyl ester; or a C₅₋₂₀aryl-C₁₋₇alkyl ester (e.g., a benzyl ester; a nitrobenzyl ester); or as an amide, for example, as a methyl amide.

For example, a thiol group may be protected as a thioether (—SR), for example, as: a benzyl thioether; an acetamidomethyl ether (—S—CH₂NHC(═O)CH₃).

### Prodrugs

It may be convenient or desirable to prepare, purify, and/or handle the compound in the form of a prodrug. The term “prodrug,” as used herein, pertains to a compound which, when metabolised (e.g., in vivo), yields the desired active compound. Typically, the prodrug is inactive, or less active than the desired active compound, but may provide advantageous handling, administration, or metabolic properties.

For example, some prodrugs are esters of the active compound (e.g., a physiologically acceptable metabolically labile ester). During metabolism, the ester group (—C(═O)OR) is cleaved to yield the active drug. Such esters may be formed by esterification, for example, of any of the carboxylic acid groups (—C(═O)OH) in the parent compound, with, where appropriate, prior protection of any other reactive groups present in the parent compound, followed by deprotection if required.

Also, some prodrugs are activated enzymatically to yield the active compound, or a compound which, upon further chemical reaction, yields the active compound (for example, as in ADEPT, GDEPT, LIDEPT, etc.). For example, the prodrug may be a sugar derivative or other glycoside conjugate, or may be an amino acid ester derivative.

### Chemical Synthesis

Several methods for the chemical synthesis of AITZ compounds of the present invention are described herein. These and/or other well known methods may be modified and/or adapted in known ways in order to facilitate the synthesis of additional compounds within the scope of the present invention.

### Compositions

One aspect of the present invention pertains to a composition (e.g., a pharmaceutical composition) comprising an AITZ compound, as described herein, and a pharmaceutically acceptable carrier, diluent, or excipient.

Another aspect of the present invention pertains to a method of preparing a composition (e.g., a pharmaceutical composition) comprising admixing an AITZ compound, as described herein, and a pharmaceutically acceptable carrier, diluent, or excipient.

### Uses

The AITZ compounds, as described herein, are useful, for example, in the treatment of disorders (e.g., diseases) that are ameliorated by the inhibition of 11β-hydroxysteroid dehydrogenase type 1 (11β-HSD1), as described herein.

### Use in Methods of Inhibiting 11β-Hydroxysteroid Dehydrogenase Type 1 (11β-HSD1)

One aspect of the present invention pertains to a method of inhibiting 11β-hydroxysteroid dehydrogenase type 1 in a cell, in vitro or in vivo, comprising contacting the cell with an effective amount of an AITZ compound, as described herein.

Suitable assays for determining 11β-hydroxysteroid dehydrogenase type 1 inhibition are described herein and/or are known in the art.

In one embodiment, the method is performed in vitro.

In one embodiment, the method is performed in vivo.

In one embodiment, the AITZ compound is provided in the form of a pharmaceutically acceptable composition.

Any type of cell may be treated, including but not limited to, adipose, lung, gastrointestinal (including, e.g., bowel, colon), breast (mammary), ovarian, prostate, liver (hepatic), kidney (renal), bladder, pancreas, brain, and skin.

One of ordinary skill in the art is readily able to determine whether or not a candidate compound inhibits 11β-hydroxysteroid dehydrogenase type 1. For example, suitable assays are described herein.

For example, a sample of cells may be grown in vitro and a compound brought into contact with said cells, and the effect of the compound on those cells observed. As an example of “effect,” the morphological status of the cells (e.g., alive or dead, etc.) may be determined. Where the compound is found to exert an influence on the cells, this may be used as a prognostic or diagnostic marker of the efficacy of the compound in methods of treating a patient carrying cells of the same cellular type.

### Use in Methods of Therapy

Another aspect of the present invention pertains to an AITZ compound, as described herein, for use in a method of treatment of the human or animal body by therapy.

### Use in the Manufacture of Medicaments

Another aspect of the present invention pertains to use of an AITZ compound, as described herein, in the manufacture of a medicament for use in treatment.

In one embodiment, the medicament comprises the AITZ compound.

### Methods of Treatment

Another aspect of the present invention pertains to a method of treatment comprising administering to a patient in need of treatment a therapeutically effective amount of an AITZ compound, as described herein, preferably in the form of a pharmaceutical composition.

### Disorders Treated—Disorders Ameliorated by the Inhibition of 11β-Hydroxysteroid Dehydroqenase Type 1

In one embodiment (e.g., of use in methods of therapy, of use in the manufacture of medicaments, of methods of treatment), the treatment is treatment or prevention of a disorder (e.g., a disease) that is ameliorated by the inhibition of 11β-hydroxysteroid dehydrogenase type 1.

Disorders Treated—Disorders characterised by Up-Regulation of 11β-HSD1 etc.

In one embodiment (e.g., of use in methods of therapy, of use in the manufacture of medicaments, of methods of treatment), the treatment is treatment or prevention of a disorder (e.g., a disease) that is characterised by one or more of: up-regulation of 11β-HSD1; up-regulation of glucocorticoid receptor mediated pathways; elevated PEPCK levels; other biochemical markers pertaining to glucocorticoid excess and insulin resistance.

### Disorders Treated

In one embodiment (e.g., of use in methods of therapy, of use in the manufacture of medicaments, of methods of treatment), the treatment is treatment or prevention of one or more of the following:

(1) Cushing's syndrome;
(2) type 2 diabetes and impaired glucose tolerance;
(3) insulin resistance syndromes such as myotonic dystrophy, Prader Willi, lipodystrophies, gastrointestinal diabetes, etc.;
(4) obesity and being overweight;
(5) lipid disorders;
(6) atherosclerosis and its sequelae, including myocardial infarction and peripheral vascular disease;

### (7) Metabolic Syndrome;

(8) steatohepatitis/fatty liver;
(9) cognitive impairment in type 2 diabetes, glucose intolerance and ageing, and in psychotic disorders and pre-schizophrenia;
(10) dementias such as Alheimer's disease, multi-infarct dementia, dementia with Lewy bodies, fronto-temporal dementia (including Pick's disease), progressive supranuclear palsy, Korsakoffs syndrome, Binswanger's disease, HIV-associated dementia, Creutzfeldt-Jakob disease (CJD), multiple sclerosis, motor neurone disease, Parkinson's disease, Huntington's disease, Niemann-Pick disease type C, normal pressure hydrocephalus, and Down's syndrome;
(11) mild cognitive impairment (cognitive impairment, no dementia);
(12) β-cell dysfunction in pancreatic disease;
(13) glaucoma;
(14) anxiety;
(15) depression and other affective disorders; typical (melancholic) and atypical depression; dysthymia; post-partum depression; bipolar affective disorder; drug-induced affective disorders; anxiety; posttraumatic stress disorder; panic; phobias;
(16) delirium and acute confusional state;
(17) inflammatory disease;
(18) osteoporosis;
(19) myocardial infarction, for example, to prevent left ventricular dysfunction after myocardial infarction; and
(20) stroke, for example, to limit ischaemic neuronal loss after cardiovascular accident.

In one embodiment (e.g., of use in methods of therapy, of use in the manufacture of medicaments, of methods of treatment), the treatment is treatment or prevention of one or more of the following:

(1) hyperglycaemia;
(2) glucose intolerance and impaired glucose tolerance;
(3) insulin resistance;
(4) hyperlipidaemia;
(5) hypertriglyceridaemia;
(6) hypercholesterolaemia;
(7) low HDL levels;
(8) high LDL levels;
(9) vascular restenosis;
(10) abdominal obesity;
(11) neurodegenerative disease;
(12) retinopathy;
(13) neuropathy;
(14) hypertension; and
(15) other diseases where insulin resistance is a component.

In one embodiment (e.g., of use in methods of therapy, of use in the manufacture of medicaments, of methods of treatment), the treatment is treatment or prevention of an adverse effect of glucocorticoids used to treat inflammatory diseases, such as asthma, chronic obstructive pulmonary disease, skin diseases, rheumatoid arthritis and other arthropathies, inflammatory bowel disease, and giant cell arthritis/polymyalgia rheumatica.

In one embodiment (e.g., of use in methods of therapy, of use in the manufacture of medicaments, of methods of treatment), the treatment is treatment or prevention of metabolic syndrome, which includes disorders such as type 2 diabetes and obesity, and associated disorders including insulin resistance, hypertension, lipid disorders and cardiovascular disorders such as ischaemic (coronary) heart disease.

In one embodiment (e.g., of use in methods of therapy, of use in the manufacture of medicaments, of methods of treatment), the treatment is treatment or prevention of a CNS disorder (e.g., a CNS disease) such as mild cognitive impairment and early dementia, including Alzheimer's disease.

### Treatment

The term “treatment,” as used herein in the context of treating a disorder, pertains generally to treatment and therapy, whether of a human or an animal (e.g., in veterinary applications), in which some desired therapeutic effect is achieved, for example, the inhibition of the progress of the disorder, and includes a reduction in the rate of progress, a halt in the rate of progress, alleviatiation of symptoms of the disorder, amelioration of the disorder, and cure of the disorder. Treatment as a prophylactic measure (i.e., prophylaxis) is also included. For example, use with patients who have not yet developed the disorder, but who are at risk of developing the disorder, is encompassed by the term “treatment.”

For example, treatment includes the prophylaxis of metabolic syndrome, reducing the incidence of metabolic syndrome, alleviating the symptoms of metabolic syndrome, etc.

The term “therapeutically-effective amount,” as used herein, pertains to that amount of a compound, or a material, composition or dosage form comprising a compound, which is effective for producing some desired therapeutic effect, commensurate with a reasonable benefit/risk ratio, when administered in accordance with a desired treatment regimen.

### Combination Therapies

The term “treatment” includes combination treatments and therapies, in which two or more treatments or therapies are combined, for example, sequentially or simultaneously. For example, the compounds described herein may also be used in combination therapies, e.g., in conjunction with other agents. Examples of treatments and therapies include, but are not limited to, chemotherapy (the administration of active agents, including, e.g., drugs, antibodies (e.g., as in immunotherapy), prodrugs (e.g., as in photodynamic therapy, GDEPT, ADEPT, etc.); surgery; radiation therapy; photodynamic therapy; gene therapy; and controlled diets.

One aspect of the present invention pertains to a compound as described herein, in combination with one or more (e.g., 1, 2, 3, 4, etc.) additional therapeutic agents, as described below.

The particular combination would be at the discretion of the physician who would select dosages using his common general knowledge and dosing regimens known to a skilled practitioner.

The agents (i.e., the compound described herein, plus one or more other agents) may be administered simultaneously or sequentially, and may be administered in individually varying dose schedules and via different routes. For example, when administered sequentially, the agents can be administered at closely spaced intervals (e.g., over a period of 5-10 minutes) or at longer intervals (e.g., 1, 2, 3, 4 or more hours apart, or even longer periods apart where required), the precise dosage regimen being commensurate with the properties of the therapeutic agent(s).

The agents (i.e., the compound described here, plus one or more other agents) may be formulated together in a single dosage form, or alternatively, the individual agents may be formulated separately and presented together in the form of a kit, optionally with instructions for their use.

Examples of additional agents/therapies that may be co-administered/combined with treatment with the AITZ compounds described herein include the following:

(1) insulin and insulin analogues;
(2) insulin sensitising agents, for example: PPAR-γ agonists; PPAR-αagonists; PPAR-α/γ dual agonists; biguanides;
(3) incretin and incretin mimetics;
(4) sulfonylureas and other insulin secretogogues;
(5) α-glucosidase inhibitors;
(6) glucagon receptor antagonists;
(7) GLP-1, GLP-1 analogues, and GLP-receptor agonists;
(8) GIP, GIP mimetics, and GIP receptor agonists;
(9) PACAP, PACAP mimetics, and PACAP receptor 3 agonists;
(10) agents that suppress hepatic glucose output, such as metformin;
(11) agents designed to reduce the absorption of glucose from the intestine, such as acarbose;
(12) phosphotyrosine phosphatase 1B inhibitors;
(13) glucose 6-phosphatase inhibitors;
(14) glucokinase activators;
(15) glycogen phosphorylase inhibitors;
(16) fructose 1,6-biphosphatase inhibitors;
(17) glutamine:fructose-6-phosphate amidotransferase inhibitors;
(18) anti-obesity agents, including: orilistat, sibutramine, fenfluramine, phentermine, dexfenfluramine, cannabinoid CB1 receptor antagonists or inverse agonists such as rimonobant, ghrelin antagonists, oxyntomodulin, neuropeptide Y1 or Y5 antagonists, 5-HT_{1B} receptor agonists, 5-HT_{2C} receptor agonists, 5-H1_{1B/2C} receptor dual agonists, melanocortin receptor agonists, and melanin-concentrating hormone receptor antagonists;
(19) anti-dyslipidaemia agents, including: HMG-CoA reductase inhibitors, PPAR-α agonists, PPAR-α/γ dual agonists, bile acid sequestrants, ileal bile acid absorption inhibitors, acyl CoA:cholesterol acyltransferase inhibitors, cholesterol absorption inhibitors, cholesterol ester transfer protein inhibitors, nicotinyl alcohol and its analogues, and anti-oxidants;
(20) anti-inflammatory agents, including: non-steroidal anti-inflammatory drugs such as aspirin; and steroidal anti-inflammatory agents such as hydrocortisone and dexamethasone;
(21) anti-hypertensive agents, including: β-blockers such as atenolol and inderal; calcium antagonists such as nifedipine; ACE inhibitors such as lisinopril, aptopril and captopril; angiotensin receptor antagonists such as candesartan, losartan and cilexetil; diuretic agents such as furosemide and benzthiazide; α-antagonists; centrally acting agents such as clonidine, methyl dopa, and indapamide; and vasodilators such as hydralazine;
(22) dipeptidyl peptidase IV (DPP-IV) inhibitors such as sitagliptin and saxagliptin;
(23) acetylcholinesterase inhibitors, including: donezepil hydrochloride, rivastigmine and galanthamine;
(24) NMDA receptor blockers, including memantine hydrochloride;
(25) Histamine H3 antagonists;
(26) 5-HT₆ receptor antagonists;
(27) α7 receptor agonists; and
(28) γ-secretase modulators, including tarenflurbil.

### Other Uses

The AITZ compounds described herein may also be used as cell culture additives to inhibit 11β-hydroxysteroid dehydrogenase type 1 (11β-HSD1), etc.

The AITZ compounds described herein may also be used as part of an in vitro assay, for example, in order to determine whether a candidate host is likely to benefit from treatment with the compound in question.

The AITZ compounds described herein may also be used as a standard, for example, in an assay, in order to identify other active compounds, other 11β-hydroxysteroid dehydrogenase type 1 (11β-HSD1) inhibitors, etc.

### Kits

One aspect of the invention pertains to a kit comprising (a) an AITZ compound as described herein, or a composition comprising an AITZ compound as described herein, e.g., preferably provided in a suitable container and/or with suitable packaging; and (b) instructions for use, e.g., written instructions on how to administer the compound or composition.

The written instructions may also include a list of indications for which the active ingredient is a suitable treatment.

### Routes of Administration

The AITZ compound or pharmaceutical composition comprising the AITZ compound may be administered to a subject by any convenient route of administration, whether systemically/peripherally or topically (i.e., at the site of desired action).

Routes of administration include, but are not limited to, oral (e.g., by ingestion); buccal; sublingual; transdermal (including, e.g., by a patch, plaster, etc.); transmucosal (including, e.g., by a patch, plaster, etc.); intranasal (e.g., by nasal spray); ocular (e.g., by eyedrops); pulmonary (e.g., by inhalation or insufflation therapy using, e.g., via an aerosol, e.g., through the mouth or nose); rectal (e.g., by suppository or enema); vaginal (e.g., by pessary); parenteral, for example, by injection, including subcutaneous, intradermal, intramuscular, intravenous, intraarterial, intracardiac, intrathecal, intraspinal, intracapsular, subcapsular, intraorbital, intraperitoneal, intratracheal, subcuticular, intraarticular, subarachnoid, and intrasternal; by implant of a depot or reservoir, for example, subcutaneously or intramuscularly.

### The Subject/Patient

The subject/patient may be a chordate, a vertebrate, a mammal, a placental mammal, a marsupial (e.g., kangaroo, wombat), a rodent (e.g., a guinea pig, a hamster, a rat, a mouse), murine (e.g., a mouse), a lagomorph (e.g., a rabbit), avian (e.g., a bird), canine (e.g., a dog), feline (e.g., a cat), equine (e.g., a horse), porcine (e.g., a pig), ovine (e.g., a sheep), bovine (e.g., a cow), a primate, simian (e.g., a monkey or ape), a monkey (e.g., marmoset, baboon), an ape (e.g., gorilla, chimpanzee, orangutang, gibbon), or a human.

Furthermore, the subject/patient may be any of its forms of development, for example, a foetus.

In one preferred embodiment, the subject/patient is a human.

### Formulations

While it is possible for the AITZ compound to be administered alone, it is preferable to present it as a pharmaceutical formulation (e.g., composition, preparation, medicament) comprising at least one AITZ compound, as described herein, together with one or more other pharmaceutically acceptable ingredients well known to those skilled in the art, including, but not limited to, pharmaceutically acceptable carriers, diluents, excipients, adjuvants, fillers, buffers, preservatives, anti-oxidants, lubricants, stabilisers, solubilisers, surfactants (e.g., wetting agents), masking agents, colouring agents, flavouring agents, and sweetening agents. The formulation may further comprise other active agents, for example, other therapeutic or prophylactic agents.

Thus, the present invention further provides pharmaceutical compositions, as defined above, and methods of making a pharmaceutical composition comprising admixing at least one AITZ compound, as described herein, together with one or more other pharmaceutically acceptable ingredients well known to those skilled in the art, e.g., carriers, diluents, excipients, etc. If formulated as discrete units (e.g., tablets, etc.), each unit contains a predetermined amount (dosage) of the compound.

The term “pharmaceutically acceptable,” as used herein, pertains to compounds, ingredients, materials, compositions, dosage forms, etc., which are, within the scope of sound medical judgment, suitable for use in contact with the tissues of the subject in question (e.g., human) without excessive toxicity, irritation, allergic response, or other problem or complication, commensurate with a reasonable benefit/risk ratio. Each carrier, diluent, excipient, etc. must also be “acceptable” in the sense of being compatible with the other ingredients of the formulation.

Suitable carriers, diluents, excipients, etc. can be found in standard pharmaceutical texts, for example, Remington's Pharmaceutical Sciences, 18th edition, Mack Publishing Company, Easton, Pa., 1990; and Handbook of Pharmaceutical Excipients, 5th edition, 2005.

The formulations may be prepared by any methods well known in the art of pharmacy. Such methods include the step of bringing into association the compound with a carrier which constitutes one or more accessory ingredients. In general, the formulations are prepared by uniformly and intimately bringing into association the compound with carriers (e.g., liquid carriers, finely divided solid carrier, etc.), and then shaping the product, if necessary.

The formulation may be prepared to provide for rapid or slow release; immediate, delayed, timed, or sustained release; or a combination thereof.

Formulations may suitably be in the form of liquids, solutions (e.g., aqueous, non-aqueous), suspensions (e.g., aqueous, non-aqueous), emulsions (e.g., oil-in-water, water-in-oil), elixirs, syrups, electuaries, mouthwashes, drops, tablets (including, e.g., coated tablets), granules, powders, losenges, pastilles, capsules (including, e.g., hard and soft gelatin capsules), cachets, pills, ampoules, boluses, suppositories, pessaries, tinctures, gels, pastes, ointments, creams, lotions, oils, foams, sprays, mists, or aerosols.

Formulations may suitably be provided as a patch, adhesive plaster, bandage, dressing, or the like which is impregnated with one or more compounds and optionally one or more other pharmaceutically acceptable ingredients, including, for example, penetration, permeation, and absorption enhancers. Formulations may also suitably be provided in the form of a depot or reservoir.

The compound may be dissolved in, suspended in, or admixed with one or more other pharmaceutically acceptable ingredients. The compound may be presented in a liposome or other microparticulate which is designed to target the compound, for example, to blood components or one or more organs.

ForMulations suitable for oral administration (e.g., by ingestion) include liquids, solutions (e.g., aqueous, non-aqueous), suspensions (e.g., aqueous, non-aqueous), emulsions (e.g., oil-in-water, water-in-oil), elixirs, syrups, electuaries, tablets, granules, powders, capsules, cachets, pills, ampoules, boluses.

Formulations suitable for buccal administration include mouthwashes, losenges, pastilles, as well as patches, adhesive plasters, depots, and reservoirs. Losenges typically comprise the compound in a flavored basis, usually sucrose and acacia or tragacanth. Pastilles typically comprise the compound in an inert matrix, such as gelatin and glycerin, or sucrose and acacia. Mouthwashes typically comprise the compound in a suitable liquid carrier.

Formulations suitable for sublingual administration include tablets, losenges, pastilles, capsules, and pills.

Formulations suitable for oral transmucosal administration include liquids, solutions (e.g., aqueous, non-aqueous), suspensions (e.g., aqueous, non-aqueous), emulsions (e.g., oil-in-water, water-in-oil), mouthwashes, losenges, pastilles, as well as patches, adhesive plasters, depots, and reservoirs.

Formulations suitable for non-oral transmucosal administration include liquids, solutions (e.g., aqueous, non-aqueous), suspensions (e.g., aqueous, non-aqueous), emulsions (e.g., oil-in-water, water-in-oil), suppositories, pessaries, gels, pastes, ointments, creams, lotions, oils, as well as patches, adhesive plasters, depots, and reservoirs.

Formulations suitable for transdermal administration include gels, pastes, ointments, creams, lotions, and oils, as well as patches, adhesive plasters, bandages, dressings, depots, and reservoirs.

Tablets may be made by conventional means, e.g., compression or moulding, optionally with one or more accessory ingredients. Compressed tablets may be prepared by compressing in a suitable machine the compound in a free-flowing form such as a powder or granules, optionally mixed with one or more binders (e.g., povidone, gelatin, acacia, sorbitol, tragacanth, hydroxypropylmethyl cellulose); fillers or diluents (e.g., lactose, microcrystalline cellulose, calcium hydrogen phosphate); lubricants (e.g., magnesium stearate, talc, silica); disintegrants (e.g., sodium starch glycolate, cross-linked povidone, cross-linked sodium carboxymethyl cellulose); surface-active or dispersing or wetting agents (e.g., sodium lauryl sulfate); preservatives (e.g., methyl p-hydroxybenzoate, propyl p-hydroxybenzoate, sorbic acid); flavours, flavour enhancing agents, and sweeteners. Moulded tablets may be made by moulding in a suitable machine a mixture of the powdered compound moistened with an inert liquid diluent. The tablets may optionally be coated or scored and may be formulated so as to provide slow or controlled release of the compound therein using, for example, hydroxypropylmethyl cellulose in varying proportions to provide the desired release profile. Tablets may optionally be provided with a coating, for example, to affect release, for example an enteric coating, to provide release in parts of the gut other than the stomach.

Ointments are typically prepared from the compound and a paraffinic or a water-miscible ointment base.

Creams are typically prepared from the compound and an oil-in-water cream base. If desired, the aqueous phase of the cream base may include, for example, at least about 30% w/w of a polyhydric alcohol, i.e., an alcohol having two or more hydroxyl groups such as propylene glycol, butane-1,3-diol, mannitol, sorbitol, glycerol and polyethylene glycol and mixtures thereof. The topical formulations may desirably include a compound which enhances absorption or penetration of the compound through the skin or other affected areas. Examples of such dermal penetration enhancers include dimethylsulfoxide and related analogues.

Emulsions are typically prepared from the compound and an oily phase, which may optionally comprise merely an emulsifier (otherwise known as an emulgent), or it may comprise a mixture of at least one emulsifier with a fat or an oil or with both a fat and an oil. Preferably, a hydrophilic emulsifier is included together with a lipophilic emulsifier which acts as a stabiliser. It is also preferred to include both an oil and a fat. Together, the emulsifier(s) with or without stabiliser(s) make up the so-called emulsifying wax, and the wax together with the oil and/or fat make up the so-called emulsifying ointment base which forms the oily dispersed phase of the cream formulations.

Suitable emulgents and emulsion stabilisers include Tween 60, Span 80, cetostearyl alcohol, myristyl alcohol, glyceryl monostearate and sodium lauryl sulfate. The choice of suitable oils or fats for the formulation is based on achieving the desired cosmetic properties, since the solubility of the compound in most oils likely to be used in pharmaceutical emulsion formulations may be very low. Thus the cream should preferably be a non-greasy, non-staining and washable product with suitable consistency to avoid leakage from tubes or other containers. Straight or branched chain, mono- or dibasic alkyl esters such as di-isoadipate, isocetyl stearate, propylene glycol diester of coconut fatty acids, isopropyl myristate, decyl oleate, isopropyl palmitate, butyl stearate, 2-ethylhexyl palmitate or a blend of branched chain esters known as Crodamol CAP may be used, the last three being preferred esters. These may be used alone or in combination depending on the properties required. Alternatively, high melting point lipids such as white soft paraffin and/or liquid paraffin or other mineral oils can be used.

Formulations suitable for intranasal administration, where the carrier is a liquid, include, for example, nasal spray, nasal drops, or by aerosol administration by nebuliser, include aqueous or oily solutions of the compound.

Formulations suitable for intranasal administration, where the carrier is a solid, include, for example, those presented as a coarse powder having a particle size, for example, in the range of about 20 to about 500 microns which is administered in the manner in which snuff is taken, i.e., by rapid inhalation through the nasal passage from a container of the powder held close up to the nose.

Formulations suitable for pulmonary administration (e.g., by inhalation or insufflation therapy) include those presented as an aerosol spray from a pressurised pack, with the use of a suitable propellant, such as dichlorodifluoromethane, trichlorofluoromethane, dichoro-tetrafluoroethane, carbon dioxide, or other suitable gases.

Formulations suitable for ocular administration include eye drops wherein the compound is dissolved or suspended in a suitable carrier, especially an aqueous solvent for the compound.

Formulations suitable for rectal administration may be presented as a suppository with a suitable base comprising, for example, natural or hardened oils, waxes, fats, semi-liquid or liquid polyols, for example, cocoa butter or a salicylate; or as a solution or suspension for treatment by enema.

Formulations suitable for vaginal administration may be presented as pessaries, tampons, creams, gels, pastes, foams or spray formulations containing in addition to the compound, such carriers as are known in the art to be appropriate.

Formulations suitable for parenteral administration (e.g., by injection), include aqueous or non-aqueous, isotonic, pyrogen-free, sterile liquids (e.g., solutions, suspensions), in which the compound is dissolved, suspended, or otherwise provided (e.g., in a liposome or other microparticulate). Such liquids may additionally contain other pharmaceutically acceptable ingredients, such as anti-oxidants, buffers, preservatives, stabilisers, bacteriostats, suspending agents, thickening agents, and solutes which render the formulation isotonic with the blood (or other relevant bodily fluid) of the intended recipient.

Examples of excipients include, for example, water, alcohols, polyols, glycerol, vegetable oils, and the like. Examples of suitable isotonic carriers for use in such formulations include Sodium Chloride Injection, Ringer's Solution, or Lactated Ringer's Injection.

Typically, the concentration of the compound in the liquid is from about 1 ng/ml to about 10 μg/ml, for example from about 10 ng/ml to about 1 μg/ml. The formulations may be presented in unit-dose or multi-dose sealed containers, for example, ampoules and vials, and may be stored in a freeze-dried (lyophilised) condition requiring only the addition of the sterile liquid carrier, for example water for injections, immediately prior to use. Extemporaneous injection solutions and suspensions may be prepared from sterile powders, granules, and tablets.

### Dosage

It will be appreciated by one of skill in the art that appropriate dosages of the AITZ compounds, and compositions comprising the AITZ compounds, can vary from patient to patient. Determining the optimal dosage will generally involve the balancing of the level of therapeutic benefit against any risk or deleterious side effects. The selected dosage level will depend on a variety of factors including, but not limited to, the activity of the particular AITZ compound, the route of administration, the time of administration, the rate of excretion of the AITZ compound, the duration of the treatment, other drugs, compounds, and/or materials used in combination, the severity of the disorder, and the species, sex, age, weight, condition, general health, and prior medical history of the patient. The amount of AITZ compound and route of administration will ultimately be at the discretion of the physician, veterinarian, or clinician, although generally the dosage will be selected to achieve local concentrations at the site of action which achieve the desired effect without causing substantial harmful or deleterious side-effects.

Administration can be effected in one dose, continuously or intermittently (e.g., in divided doses at appropriate intervals) throughout the course of treatment. Methods of determining the most effective means and dosage of administration are well known to those of skill in the art and will vary with the formulation used for therapy, the purpose of the therapy, the target cell(s) being treated, and the subject being treated. Single or multiple administrations can be carried out with the dose level and pattern being selected by the treating physician, veterinarian, or clinician.

In general, a suitable dose of the AITZ compound is in the range of about 10 μg to about 250 mg (more typically about 100 μg to about 25 mg) per kilogram body weight of the subject per day. Where the compound is a salt, an ester, an amide, a prodrug, or the like, the amount administered is calculated on the basis of the parent compound and so the actual weight to be used is increased proportionately.

### EXAMPLES

The following examples are provided solely to illustrate the present invention and are not intended to limit the scope of the invention, as described herein.

### Analytical Method 1:

The system consisted of a Hewlett Packard HP1100 LC system and a Higgins Clipeus 5 μm C18 100×3.0 mm column. Detection was achieved using a Micromass ZQ quadrupole electrospray (positive and negative ion), a UV detector at 254 nm and a Sedex ELS 85 evaporative light scattering detector. Mobile Phase A: 0.1% aqueous formic acid, Mobile Phase B: 0.1% formic acid in MeCN. Flow rate 1 mL/min: Gradient: 0-1 min 5% B; 1-15 min 5-95% B; 15-20 min 95% B; 20-22 min 95-5% B; 22-25 min 95% B.

### Analytical Method 2:

The system consisted of a Finnigan AQA single quadrupole mass spectrometer linked to a Hewlett Packard 1050 LC system with UV diode array detector and autosampler and using a Luna 3 μm C18(2) 30×4.6 mm column or equivalent. The spectrometer had an electrospray source operating in positive ion mode. Additional detection was achieved using a Sedex 65 evaporative light scattering detector. Mobile Phase A: 0.1% aqueous formic acid, Mobile Phase B: 0.1% formic acid in MeCN. Flow rate 2 mL/min: Gradient 0-0.5 min 5% B; 0.5-4.5 min 5-95% B; 4.5-5.5 95% B; 5.5-6.0 min 95-5% B.

### Analytical Method 3:

The system consisted of a Finnigan AQA single quadrupole mass spectrometer linked to a Hewlett Packard 1050 LC system with UV diode array detector and autosampler and using a Luna 3 micron C18(2) 30×4.6 mm column or equivalent. The spectrometer had an electrospray source operating in positive ion mode. Additional detection was achieved using a Sedex 65 evaporative light scattering detector. Mobile Phase A: 0.1% aqueous formic acid, Mobile Phase B: 0.1% formic acid in methanol. Flow rate 2 mL/min: Gradient 0-0.5 min 5% B; 0.5-4.5 min 5-95% B; 4.5-5.5 95% B; 5.5-6.0 min 95-5% B.

### Analytical Method 4:

The system consisted of a Hewlett Packard HP1100 LC system and a Higgins Clipeus 5 μm C18 100×3.0 mm column. Detection was achieved using a Micromass ZQ quadrupole electrospray (positive and negative ion), a UV detector at 254 nm and a Sedex ELS 85 evaporative light scattering detector. Mobile Phase A: 0.1% aqueous formic acid, Mobile Phase B: 0.1% formic acid in methanol. Flow rate 1 mL/min: Gradient: 0-1 min 15% B; 1-13 min 15-95% B; 13-20 min 95% B; 20-22 min 95-15% B; 22-25 min 15% B.

### Analytical Method 5:

The system consisted of an Agilent 1200 HPLC and mass spectrometer system and an Agilent Scalar 5 μm C18 50×4.6 mm column. Detection was achieved using an electrospray ionization source (positive or negative ion), a UV detector at 254 nm. Mobile Phase A: 0.1% aqueous formic acid, Mobile Phase B: 0.1% formic acid in MeCN. Flow rate 2.5 mL/min: Gradient: 0-0.1 min 5% B; 0.1-5 min 5-95% B; 5-5.5 min 95% B; 5.5-5.6 min 95% B, flow increased to 3.5 mL/min; 5.6-6.6 95% B; 6.6-6.75 min 95-5% B; 6.75-6.9 min 5% B; 6.9-7 min 5% B, flow rate reduced to 2.5 mL/min.

### Analytical Method 6:

The system consisted of a Waters HPLC and mass spectrometer system and an Agilent Scalar 5 μm C18 50×4.6 mm column. Detection was achieved using an electrospray ionization source (positive or negative ion), a UV detector at 254 nm and 215 nm. Mobile Phase A: 0.1% aqueous formic acid, Mobile Phase B: 0.1% formic acid in MeCN. Flow rate 2.5 mL/min: Gradient: 0-0.1 min 5% B; 0.1-5 min 5-95% B; 5-5.5 min 95% B; 5.5-5.6 min 95% B, flow increased to 3.5 mL/min; 5.6-6.6 95% B; 6.6-6.75 min 95-5% B; 6.75-6.9 min 5% B; 6.9-7 min 5% B, flow reduced to 2.5 mL/min.

### ABBREVIATIONS

AIBN=2,2′-Azobis(2-methylpropionitrile).
DCC=N,N′-Dicyclohexylcarbodiimide.
1,2-DCE=1,2-Dichloroethane.
DCM=Dichloromethane.
DIPEA=Diisopropylethylamine.
DME=1,2-Dimethoxyethane.
DMF=Dimethylformamide.
HATU=(O-(7-Azabenzotriazol-1-yl)-N,N,N′,N′-tetramethyluroniumhexafluoro-phosphate).
HCl=Hydrochloric acid.
IMS=Industrial methylated spirit.
NBS=N-bromosuccinimide.
R.T.=retention time.
THF=Tetrahydrofuran.
s=singlet.
d=doublet.
t=triplet.
m=multiplet.
q=quartet.

Compounds were named using Autonom.

Compounds containing chiral centres were prepared as racemic mixtures, unless stated otherwise.

### Materials

Decahydroquinoline (a mixture containing both of the cis- and both of the trans-enantiomers) was obtained from the Sigma-Aldrich Corporation.

Single cis-decahydroquinoline enantiomers were prepared from 3-(2-oxo-cyclohexyl)-propionic acid and (S)-(+)-2-phenylglycinol, using the method described in Amat et al., 2006, Chem. Eur. J., Vol. 12, No. 30, pp. 7872-7881.

Compounds containing decahydroquinolyl amides were prepared as:

(a) a mixture of the cis-diastereomers (referred to as DHQ [CIS-M]) or
(b) a single enantiomer of the cis-decahydroquinoline, prepared using (S)-(+)-2-phenylglycinol as a chiral auxiliary utilising the method outlined above (referred to as DHQ [CIS-S]).

It is predicted that the single enantiomer of cis-decahydroquinoline (DHQ [CIS-S]) prepared using (S)-(+)-2-phenylglycinol as a chiral auxiliary is, or is predominantly, (4aS,8aS)-decahydroquinoline.

When thiophene-3-carboxylic acids are coupled using HATU to a mixture containing an excess of both of the cis- and both of the trans-isomers of decahydroquinoline, it was observed that the amide formed with the cis-decahydroquinoline is the major product. Decahydroquinolyl amides consisting of a mixture of cis-diastereomers (referred to as DHQ [CIS-M]) were prepared using this method.

### Synthesis 1

### 2-(3-Methyl-isothiazol-5-yl)-isoindole-1,3-dione

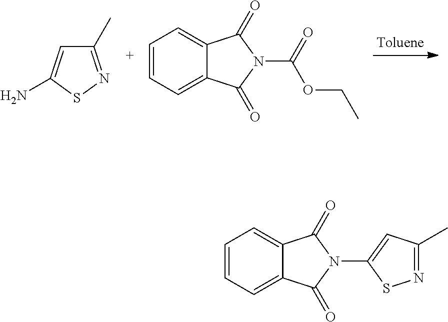

3-Methyl-isothiazol-5-ylamine hydrochloride was converted to the free base by dissolving the material in ethyl acetate and washing with a 10% solution of sodium carbonate. The organic phase was dried and evaporated to afford the free base.

3-Methyl-isothiazol-5-ylamine (4.07 g, 18.57 mmol) was combined with 1,3-dioxo-1,3-dihydro-isoindole-2-carboxylic acid ethyl ester (2.0 g, 17.52 mmol) in toluene (50 mL) and heated at 130° C. for 48 hours. The solvent was removed by evaporation then 10% HCl was added and the mixture was extracted with DCM (×3). The organic phases were dried over sodium sulfate, filtered and the solvent evaporated. The resultant solid was washed with pentane then dried overnight to afford the title compound (4.07 g), which was used without further purification. ¹H NMR (400 MHz, CHCl□-d): δ 8.00-7.96 (m, 2 H); 7.85-7.81 (m, 3H); 2.53 (s, 3H).

### Synthesis 2

### 2-(3-Bromomethyl-isothiazol-5-yl)-isoindole-1,3-dione

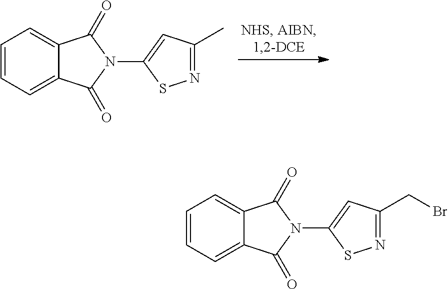

2-(3-Methyl-isothiazol-5-yl)-isoindole-1,3-dione (3.0 g, 12.28 mmol) was stirred with N-bromosuccinimide (2.3 g, 12.89 mmol) and 2,2′-azobis(2-methylpropionitrile) (0.25 g) in 1,2-DCE (100 mL) under a 150 W halogen light for 4 hours and then a further amount of 2,2′-azobis(2-methylpropionitrile) (0.15 g) together with 1,1′-azobis(cyclohexanecarbonitrile) (0.15 g) was added. The reaction mixture was stirred overnight at 90° C. then N-bromosuccinimide (2.3 g) was added together with 1,1′-azobis(cyclohexanecarbonitrile) (0.10 g) and stirring was continued for 8 hours. The reaction mixture was cooled to room temperature and quenched with sodium metabisulfite before extracting with DCM. The organic phases were dried over sodium sulfate, filtered and the solvent concentrated to about 20 mL. The resultant solution was left to stand over the weekend, during which time a solid crystallised. This was collected by filtration, washing with cyclohexane and a small quantity of DCM to afford the title compound (1.15 g). ¹H NMR (400 MHz, CHCl□-d): δ 8.12 (s, 1H); 8.04-7.97 (m, 2H); 7.88-7.82 (m, 2H); 4.56 (s, 2H).

### Synthesis 3

### 5-(1,3-Dioxo-1,3-dihydro-isoindol-2-yl)-isothiazole-3-carboxylic acid

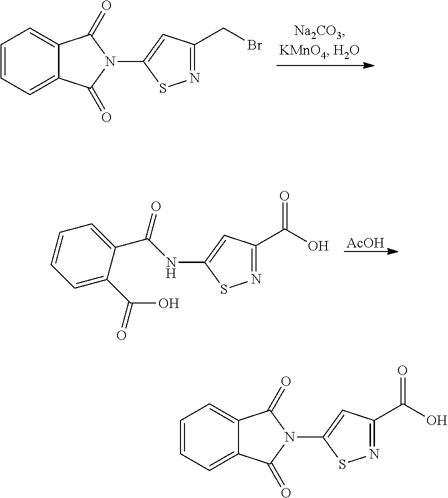

2-(3-Bromomethyl-isothiazol-5-yl)-isoindole-1,3-dione (0.50 g, 1.55 mmol) was stirred with sodium carbonate (0.175 g, 1.65 mmol) in water (10 mL) at reflux and potassium permanganate (0.32 g, 2.0 mmol) was added in small portions over 30 minutes. The reaction mixture was stirred for 30 minutes then filtered and the filtrate was evaporated to afford 5-(2-carboxy-benzoylamino)-isothiazole-3-carboxylic acid (0.79 g), which was used directly in the next reaction. This material (0.79 g) was treated with acetic acid (8 mL) and heated at 100° C. then filtered and evaporated. Water was added and the resultant solid was collected by filtration to give the title compound (0.158 g) which was used without further purification. LCMS m/z 275.28 [M+H]⁺ R.T.=2.95 mins (Analytical Method 2).

### Synthesis 4

### 2-[3-((4aS,8aS)-Octahydro-quinoline-1-carbonyl)-isothiazol-5-yl]-isoindole-1,3-dione (BB-03)

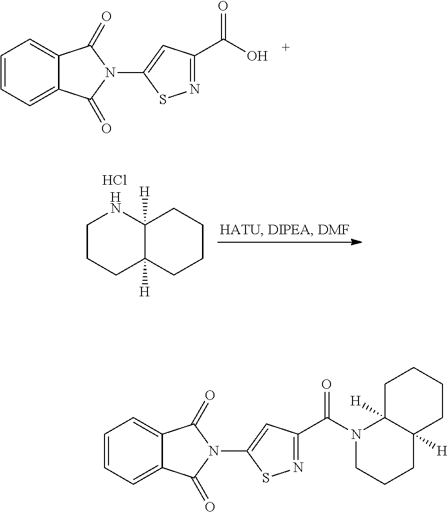

5-(1,3-Dioxo-1,3-dihydro-isoindol-2-yl)-isothiazole-3-carboxylic acid (0.158 g, 0.58 mmol) and (4aS,8aS)-decahydro-quinoline hydrochloride (0.111 g, 0.63 mmol) were dissolved in DMF (5 mL). DIPEA (0.5 mL, 2.9 mmol) was added followed by HATU (0.263 g, 0.69 mmol) and the reaction mixture stirred at room temperature for 2 hours. The reaction mixture was evaporated and the residue was dissolved in DCM then washed with 10% citric acid solution then saturated sodium bicarbonate solution. The organic phase was dried over sodium sulfate, filtered and the solvent evaporated. The residue was purified by column chromatography (eluting with 30% EtOAc in cyclohexane) to afford the title compound (0.046 g). LCMS m/z 396.10 [M+H]⁺ R.T.=12.78 mins (Analytical Method 1).

### Synthesis 5

### (5-Amino-isothiazol-3-yl)-(4aS,8aS)-octahydro-quinolin-1-yl-methanone

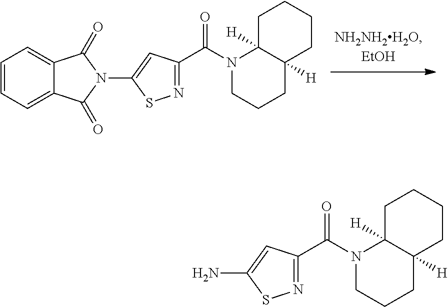

Hydrazine hydrate (60 μL, 1.23 mmol) was added to 2-[3-((4aS,8aS)-octahydro-quinoline-1-carbonyl)-isothiazol-5-yl]-isoindole-1,3-dione (0.194 g, 0.49 mmol) in ethanol (8 mL) and the reaction mixture was stirred at reflux for 1 hour. The reaction mixture was cooled and filtered then evaporated and the residue purified by column chromatography (5% methanol in DCM plus 4 drops of acetic acid) to afford the title compound (0.115 g). LCMS m/z 266.24 [M+H]⁺ R.T.=4.04 mins (Analytical Method 3).

### Synthesis 6

### (5-Bromo-isothiazol-3-yl)-(4aS,8a5)-octahydro-quinolin-1-yl-methanone

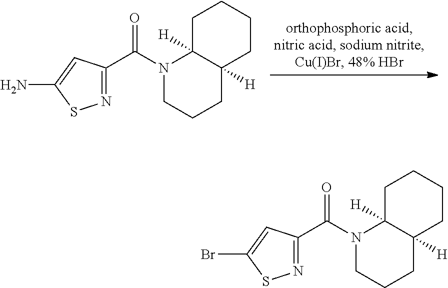

(5-Amino-isothiazol-3-yl)-(4aS,8aS)-octahydro-quinolin-1-yl-methanone (0.11 g, 0.42 mmol) was dissolved in orthophosphoric acid (2 mL) and cooled to 0° C. Nitric acid (1 mL) was added dropwise, followed by dropwise addition of a solution of sodium nitrite (0.033 g, 0.48 mmol) in the minimum amount of water, maintaining the temperature between 0-5° C. The reaction mixture was stirred at 10° C. for 30 minutes then was added dropwise to a mixture of copper (I) bromide (0.072 g, 0.50 mmol) in 48% HBr (12 mL) and the resultant mixture was stirred at room temperature for 1 hour. The pH was adjusted to 4-5 using 3 N sodium hydroxide before the mixture was extracted with DCM (×3). The organic phases were dried over sodium sulfate, filtered and the solvent evaporated to afford the title compound (0.14 g). LCMS m/z 329.17/331.18 [M+H]⁺ R.T.=4.83 mins (Analytical Method 3).

### Synthesis 7

### (4aS,8aS)-Octahydro-quinolin-1-yl-[5-(1H-pyrazol-4-yl)-isothiazol-3-yl]-methanone (BB-01)

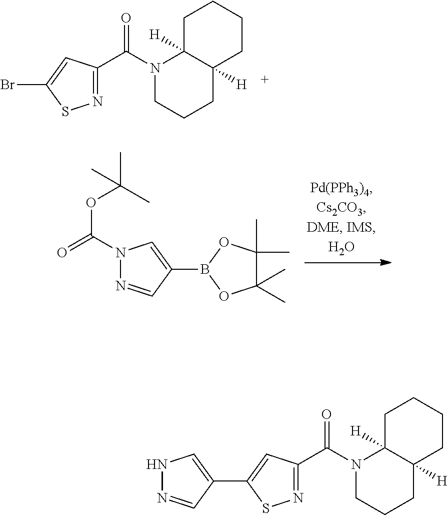

(5-Bromo-isothiazol-3-yl)-(4aS,8aS)-octahydro-quinolin-1-yl-methanone (0.034 g, 0.10 mmol) was combined with 4-(4,4,5,5-tetramethyl-[1,3,2]dioxaborolan-2-yl)-pyrazole-1-carboxylic acid tert-butyl ester (0.034 g, 0.11 mmol), tetrakis(triphenylphosphine) palladium (0) (0.012 g, 0.010 mmol), caesium carbonate (51 mg, 0.15 mmol), IMS (0.3 mL) and water (0.15 mL) in DME (1 mL) and the reaction mixture heated to 145° C. using microwave irradiation for 20 minutes. The mixture was diluted with water/sodium bicarbonate solution (1:1) and DCM then extracted with DCM (×3). The organic phases were dried over sodium sulfate, filtered and the solvent evaporated then the residue was purified by HPLC, eluting with 5%-95% methanol in water (with 0.1% formic acid) to afford the title compound (0.012 g). LCMS m/z 317.18 [M+H]⁺ R.T.=9.47 min (Analytical Method 1).

### Synthesis 8

### 5-Bromo-3-methyl-isothiazole

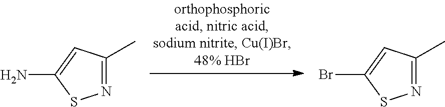

3-Methyl-isothiazol-5-ylamine hydrochloride was converted to the free base by dissolving the material in ethyl acetate and washing with a 10% solution of sodium carbonate. The organic phase was dried and evaporated to afford the free base. 3-Methyl-isothiazol-5-ylamine (6.0 g, 52.55 mmol) was dissolved in orthophosphoric acid (20 mL) and cooled to 0° C. Nitric acid (10 mL) was added dropwise, followed by dropwise addition of a solution of sodium nitrite (4.17 g, 60.44 mmol) in the minimum amount of water, maintaining the temperature between 0-5° C. The reaction mixture was stirred at 10° C. for 30 minutes then was added dropwise to a mixture of copper (I) bromide (9.5 g, 63.06 mmol) in 48% aqueous HBr (100 mL) and the resultant mixture was stirred at room temperature for 1 hour. The pH was adjusted to 6-7 using 4 N sodium hydroxide and water was added (500 mL) then a steam distillation was carried out on the rotary evaporator using water (2×500 mL). The aqueous layers were extracted with diethyl ether (×2) then the organic phases were dried over sodium sulfate, filtered and the solvent evaporated to afford the title compound (7.0 g). LCMS m/z 218.97/220.98 [M+MeCN]⁺ R.T.=3.29 min (Analytical Method 2).

### Synthesis 9

### 5-Bromo-3-bromomethyl-isothiazole

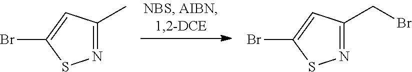

5-Bromo-3-methyl-isothiazole (3.7 g, 20.78 mmol) in 1,2-DCE (100 mL) was heated to 90° C. and N-bromosuccinimide (4.06 g, 22.86 mmol) was added as the mixture was heated. 2,2′-Azobis(2-methylpropionitrile) (0.30 g) was added and a 150 W halogen light was shone onto the reaction mixture, which was stirred overnight at 90° C. Aqueous saturated sodium bisulfite was added before extracting with DCM. The organic phases were dried over sodium sulfate, filtered and the solvent was evaporated. The residue was purified by column chromatography (eluting with cyclohexane: DCM, 2:1) to afford impure title compound (2.8 g), which was used without further purification. LCMS m/z 255.86/257.96/259.80 [M+H]⁺ R.T.=4.17/4.64 mins (Analytical Method 3).

### Synthesis 10

### (5-Bromo-isothiazol-3-yl)-[2-(4-fluoro-phenyl)-pyrrolidin-1-yl]-methanone

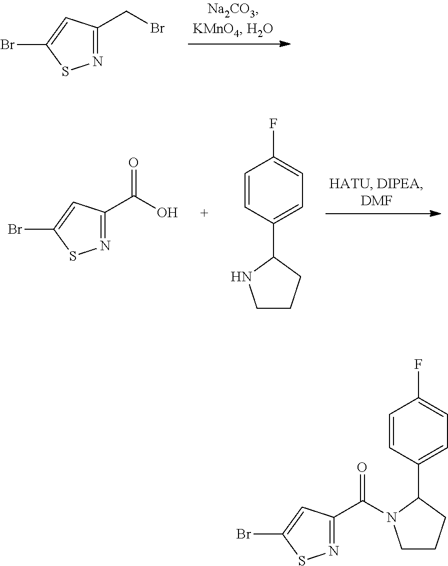

5-bromo-3-bromomethyl-isothiazole (2.8 g, 10.90 mmol) was stirred with sodium carbonate (1.27 g, 11.99 mmol) in water (60 mL) at reflux and potassium permanganate (2.24 g, 14.17 mmol) was added in small portions over 30 minutes. The reaction mixture was stirred for 30 minutes then filtered through celite and evaporated. This material was suspended in acetonitrile, filtered and evaporated then suspended in DCM and the solid collected by filtration to afford 5-bromo-isothiazole-3-carboxylic acid. This material (0.208 g, 1.0 mmol) together with 4-fluorophenylpyrrolidine (0.182 g, 1.1 mmol) and DIPEA (0.53 mL, 3.0 mmol) was dissolved in DMF (10 mL). HATU (0.42 g, 1.1 mmol) was added and the reaction mixture stirred at room temperature for 3 hours. DCM was added and the mixture was washed with saturated sodium bicarbonate solution. The organic phase was dried over sodium sulfate, filtered and the solvent evaporated. The residue was purified by column chromatography (eluting with 30% EtOAc in cyclohexane) to afford the title compound (0.221 g). LCMS m/z 355.07/357.08 [M+H]⁺ R.T.=4.59 mins (Analytical Method 3).

### Synthesis 11

### [5-(3,6-Dihydro-2H-pyran-4-yl)-isothiazol-3-yl]-[2-(4-fluoro-phenyl)-pyrrolidin-1-yl]-methanone

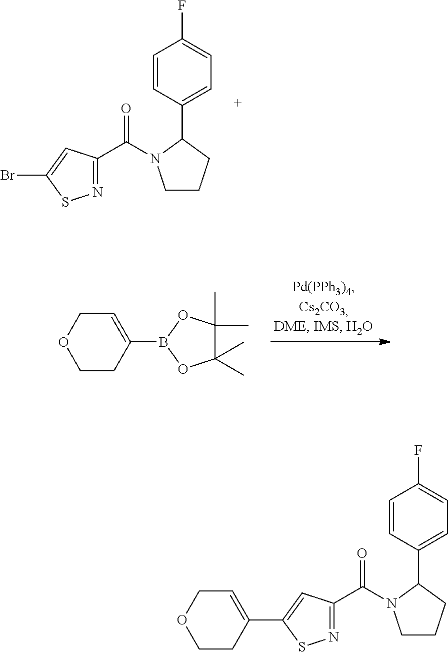

(5-Bromo-isothiazol-3-yl)-[2-(4-fluoro-phenyl)-pyrrolidin-1-yl]-methanone (0.15 g, 0.42 mmol) was combined with 4-(4,4,5,5-tetramethyl-[1,3,2]dioxaborolan-2-yl)-3,6-dihydro-2H-pyran (0.098 g, 0.47 mmol), tetrakis(triphenylphosphine) palladium (0) (0.050 g, 0.042 mmol), caesium carbonate (0.207 g, 0.63 mmol), IMS (0.90 mL) and water (0.45 mL) in DME (3 mL) and the reaction mixture heated to 120° C. using microwave irradiation for 25 minutes. The mixture was diluted with water and sodium bicarbonate solution (1:1) then extracted with DCM (×2). The organic phases were dried over sodium sulfate, filtered and the solvent evaporated then the residue was purified by column chromatography (eluting with 30% EtOAc in cyclohexane) to afford the title compound (0.143 g). LCMS m/z 359.23 [M+H]⁺ R.T.=4.46 mins (Analytical Method 3).

### Synthesis 12

### [2-(4-Fluoro-phenyl)-pyrrolidin-1-yl]-[5-(tetrahydro-pyran-4-yl)-isothiazol-3-yl]-methanone (CC-02)

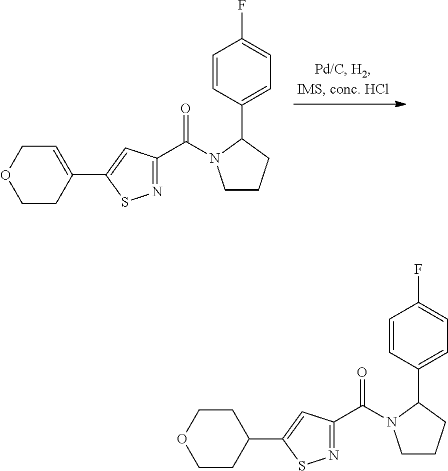

[5-(3,6-Dihydro-2H-pyran-4-yl)-isothiazol-3-yl]-[2-(4-fluoro-phenyl)-pyrrolidin-1-yl]-methanone (0.143 g, 0.40 mmol) was stirred in IMS (10 mL) with palladium on carbon (0.03 g) under an atmosphere of hydrogen for 20 hours. A further amount of palladium on carbon (0.20 g) was added and stirring continued for 48 hours when a further quantity of palladium on carbon was added (0.15 g) together with 2 drops of concentrated HCl. The reaction mixture was stirred under hydrogen for 24 hours and then the mixture was filtered through celite and evaporated. The crude residue was purified by HPLC, eluting with 20%-95% methanol in water (with 0.1% formic acid) to afford the title compound (0.033 g). LCMS m/z 361.08 [M+H]⁺ R.T.=10.74 min (Analytical Method 4).

### Synthesis 13

### [2-(4-Fluoro-phenyl)-pyrrolidin-1-yl]-[5-(1H-pyrazol-4-yl)-isothiazol-3-yl]-methanone (BB-02)

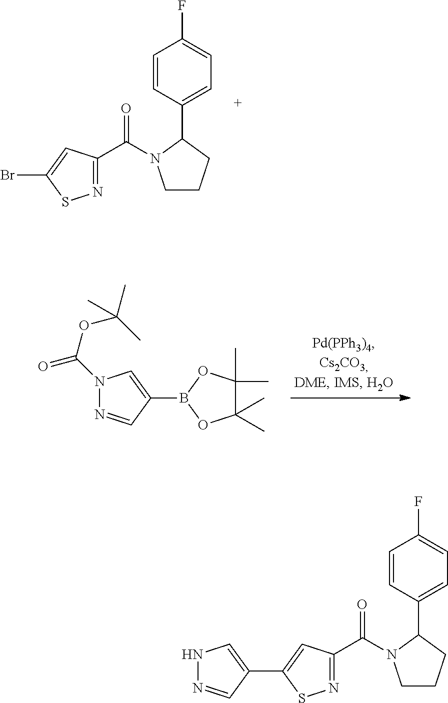

(5-Bromo-isothiazol-3-yl)-[2-(4-fluoro-phenyl)-pyrrolidin-1-yl]-methanone (0.075 g, 0.21 mmol) was combined with 4-(4,4,5,5-tetramethyl-[1,3,2]dioxaborolan-2-yl)-pyrazole-1-carboxylic acid tert-butyl ester (0.068 g, 0.23 mmol), tetrakis(triphenylphosphine) palladium (0) (0.025 g, 0.021 mmol), caesium carbonate (0.103 g, 0.325 mmol), IMS (0.60 mL) and water (0.30 mL) in DME (2 mL) and the reaction mixture heated to 145° C. using microwave irradiation for 20 minutes. The mixture was diluted with water/sodium bicarbonate solution (1:1) and DCM then extracted with DCM (×3). The organic phases were dried over sodium sulfate, filtered and the solvent evaporated then the residue was purified by HPLC, eluting with 5%-95% methanol in water (with 0.1% formic acid) to afford the title compound (0.029 g). LCMS m/z 343.14 [M+H]+ R.T.=10.17 min (Analytical Method 4).

### Synthesis 14

### (5-Bromo-isothiazol-3-yl)-(4aS,8aS)-octahydro-quinolin-1-yl-methanone

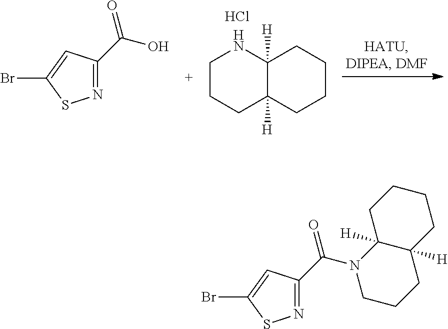

5-bromo-isothiazole-3-carboxylic acid (0.10 g, 0.48 mmol) was dissolved in DMF (5 mL) and DIPEA (0.42 mL, 2.41 mmol) was added followed by (4aS,8aS)-decahydro-quinoline hydrochloride (0.093 g, 0.53 mmol) then HATU (0.202 g, 0.53 mmol). The reaction mixture was stirred at room temperature for 1 hour 50 minutes. DCM was added and the mixture was washed with saturated sodium bicarbonate solution then the organic phase was dried over sodium sulfate, filtered and the solvent evaporated. The residue was purified by column chromatography (eluting with 30% EtOAc in cyclohexane) to afford the title compound (0.11 g). LCMS m/z 329.17/331.18 [M+H]⁺ R.T.=4.82 mins (Analytical Method 3).

### Synthesis 15

### [5-(3,6-Dihydro-2H-pyran-4-yl)-isothiazol-3-yl]-(4aS,8aS)-octahydro-quinolin-1-yl-methanone

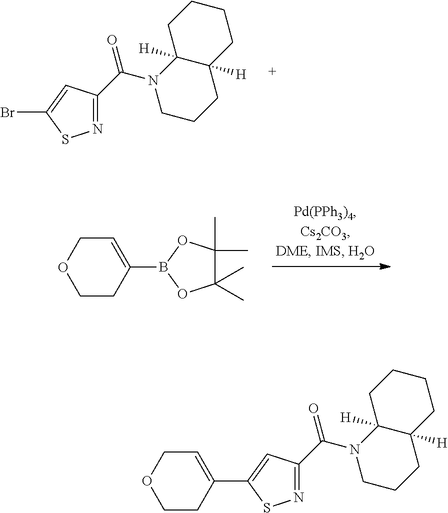

(5-Bromo-isothiazol-3-yl)-(4aS,8aS)-octahydro-quinolin-1-yl-methanone (0.11 g, 0.33 mmol) was combined with 4-(4,4,5,5-tetramethyl-[1,3,2]dioxaborolan-2-yl)-3,6-dihydro-2H-pyran (0.078 g, 0.37 mmol), tetrakis(triphenylphosphine) palladium (0) (0.039 g, 0.033 mmol), caesium carbonate (0.163 g, 0.50 mmol), IMS (0.90 mL) and water (0.45 mL) in DME (3 mL) and the reaction mixture heated to 120° C. using microwave irradiation for 25 minutes. The mixture was diluted with water and sodium bicarbonate solution (1:1) then extracted with DCM (×2). The organic phases were dried over sodium sulfate, filtered and the solvent evaporated then the residue was purified by column chromatography (eluting with 30% EtOAc in cyclohexane) to afford the title compound (0.103 g). LCMS m/z 333.32 [M+H]⁺ R.T.=4.65 mins (Analytical Method 3).

### Synthesis 16

### (4aS,8aS)-Octahydro-quinolin-1-yl-[5-(tetrahydro-pyran-4-yl)-isothiazol-3-yl]-methanone (CC-01)

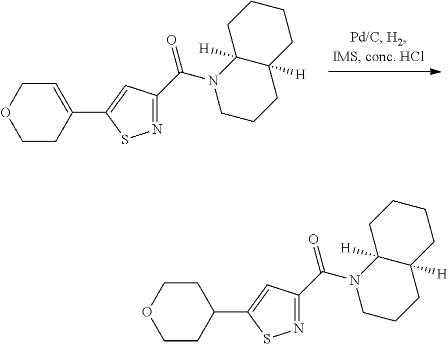

[5-(3,6-Dihydro-2H-pyran-4-yl)-isothiazol-3-yl]-(4aS,8aS)-octahydro-quinolin-1-yl-methanone (0.103 g, 0.31 mmol) was stirred in IMS (10 mL) with palladium on carbon (0.03 g) under an atmosphere of hydrogen for 20 hours. A further amount of palladium on carbon (0.20 g) was added and stirring continued for 48 hours at which point more palladium on carbon was added (0.15 g) together with 2 drops of conc. HCl. The reaction was stirred under hydrogen for 24 hours and then the mixture was filtered through celite and evaporated. The crude residue was purified by HPLC, eluting with 20%-95% methanol in water (with 0.1% formic acid) to afford the title compound (0.022 g). LCMS m/z 335.18 [M+H]+ R.T.=11.51 min (Analytical Method 4).

### Synthesis 17

### 2-(Acetylthio)-2-phenylacetic acid

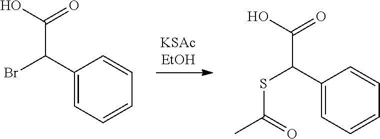

Following the procedure described in Scaffer et al., U.S. Pat. No. 5,087,631, a solution of alpha-bromophenylacetic acid (2 g; 1 eq) and potassium thioacetate (2.1 g; 2 eq) in EtOH (100 mL) was stirred at room temperature for 3 hours. The mixture was concentrated under reduced pressure to ˜10 mL, diluted with water (50 mL) and DCM (50 mL) and the layers separated. The aqueous layer was made acidic with conc. HCl (10 mL) and extracted with EtOAc (2×50 mL) to yield the title compound (1.8 g; 92%).

### Synthesis 18

### 2-Mercaptophenylacetic acid

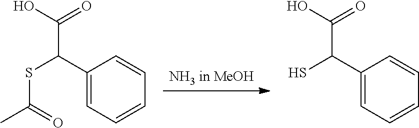

Following the procedure described in Scaffer et al., U.S. Pat. No. 5,087,631, a solution of ammonia in MeOH (7 N, 25 mL), at 0° C., was added to a solution of 2-(acetylthio)-2-phenylacetic acid (1.8 g; 1 eq) in MeOH (10 mL). The reaction mixture was allowed to warm to room temperature whilst stirring. After 1 hour, the mixture was concentrated to dryness under reduced pressure, diluted with saturated NaHCO₃ (80 mL) and EtOAc (100 mL) and the layers separated. The aqueous layer was made acidic with conc. HCl (10 mL) and extracted with EtOAc (2×70 mL) to yield the title compound (1.3 g; 90%).

### Synthesis 19

### 4-Phenyl-1,3,2-oxathiazolium-5-one

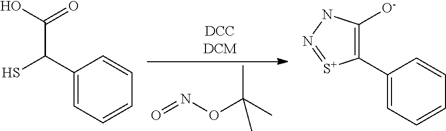

Following the procedure described in Scaffer et al., U.S. Pat. No. 5,087,631, a solution of 2-mercaptophenylacetic acid (1.3 g; 1 eq) in DCM (75 mL) was added to a solution of tert-butyl nitrite (1 g; 1.5 eq) in DCM (20 mL) at 0° C. The mixture was stirred at this temperature for 20 minutes before the addition of a solution of DCC (3.2 g, 2 eq) in DCM (20 mL). The mixture was stirred at 0° C. for 2 hours before concentrating to dryness under reduced pressure. The crude residue was purified by column chromatography (EtOAc/iso-hexane) to yield the title compound (0.36 g; 26%).

### Synthesis 20

### 5-Phenylisothiazole-4-carboxylate and 5-Phenylisothiazole-3-carboxylate

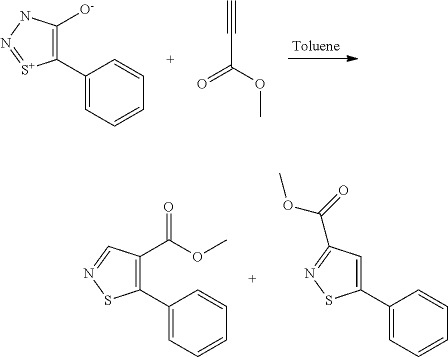

Following the procedure described in Gotthardt, H., 1971, Tetrahedron Letters, No. 17, pp. 1281-1284, a suspension of 4-phenyl-1,3,2-oxathiazolium-5-one (0.3 g, 1 eq) in toluene (5 mL) was added to methyl propiolate (745 μL, 5 eq) under a nitrogen atmosphere. The mixture was heated to 95° C. for 4 days. The crude residue was purified by column chromatography (EtOAc/iso-hexane) to yield the target compounds 5-phenylisothiazole-4-carboxylate (0.027 g, 7%) and 5-phenylisothiazole-3-carboxylate (0.011 g; 3%). LCMS m/z 220 [M+H]⁺ R.T.=3.8 min (Analytical Method 5), 93% and 90% purity, respectively.

### Synthesis 21

### 5-Phenylisothiazole-4-carboxylic acid

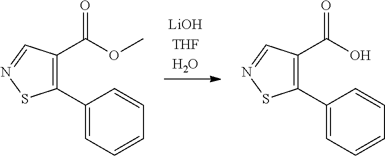

To a solution of methyl 5-phenylisothiazole-4-carboxylate (0.027 g, 1 eq) in THF (1 mL) was added a solution of LiOH (0.004 g, 3 eq) in water (350 μL). The mixture was stirred at room temperature for 3 hours, before concentrating to dryness under reduced pressure to yield the crude desired compound (0.025 g). LCMS m/z 204 [M−H]⁻ R.T.=2.59 min (Analytical Method 6).

### Synthesis 22

### 5-Phenylisothiazole-3-carboxylic acid

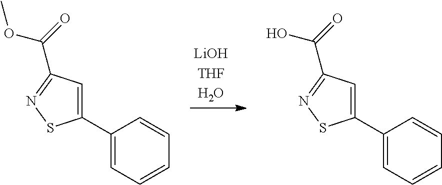

5-phenylisothiazole-3-carboxylate was subjected to the conditions used for the preparation of 5-phenylisothiazole-4-carboxylate (stirred at room temperature for only 1 hour) to yield the title compound (0.010 g; 56%). LCMS m/z 204 [M−H]⁻ R.T.=2.57 min (Analytical Method 6), 100% purity.

### Synthesis 23

### (Octahydro-quinolin-1-yl)-(5-phenyl-isothiazol-4-yl)-methanone (AA-01)

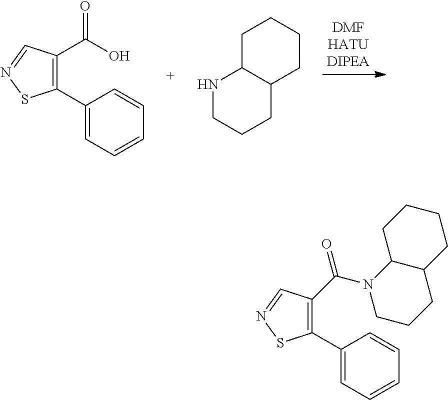

To a solution of 5-phenylisothiazole-4-carboxylic acid (0.025 g; 1 eq) in DMF (1 mL) was added HATU (0.051 g; 1.1 eq) and DIPEA (32 μL; 1.5 eq). This mixture was stirred at room temperature for 10 minutes, then a solution of the amine (54 μL; 3 eq) in DMF (1 mL) was added and the reaction mixture allowed to stir for 18 hours. The mixture was diluted with water (10 mL) and EtOAc (5 mL) and the organic layer separated, dried and evaporated to give the crude product. This was purified by column chromatography (EtOAc/iso-hexane) to give the title compound (0.015 g; 38%). LCMS m/z 327 [M+H]⁺ R.T.=4.3 min (Analytical Method 5), 100% purity.

### Synthesis 24

(Octahydro-quinolin-1-yl)-(5-phenyl-isothiazol-3-yl)-methanone (AA-02)

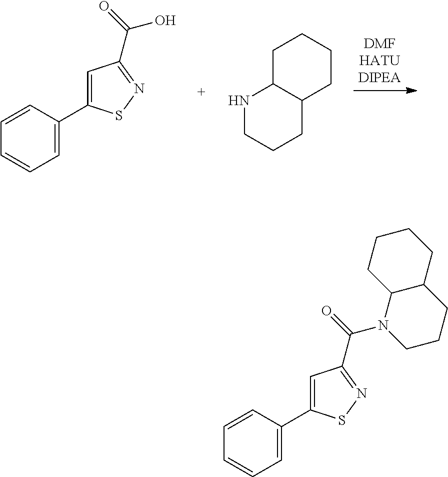

5-phenylisothiazole-3-carboxylic acid was subjected to conditions used for the preparation of (octahydro-quinolin-1-yl)-(5-phenyl-isothiazol-4-yl)-methanone to yield the title compound (0.0025 mg; 14%). LCMS m/z 327 [M+H]⁺ R.T.=4.9 min (Analytical Method 5), 100% purity.

### Biological Methods

### Cellular In Vitro 11β-HSD1 Enzyme Inhibition Assay

Compounds were assessed by a Scintillation Proximity Assay (SPA) performed according to the following protocol:

HEK293 cells were stably transfected with a construct containing the full-length gene coding for the human 11β-HSD1 enzyme to create HEK293/11β-HSD1 cells. Cells were routinely cultured in DMEM containing 10% calf foetal serum, 1% glutamine, and 1% penicillin and streptomycin. Prior to assay, cells were plated at 2×10⁴ cells/well in 96-well poly-D-Lys coated flat-bottomed microplates and incubated in 5% CO₂, 95% O₂ at 37° C. for 24 hours. The media in each well was removed immediately before assay.

Compounds to be tested were dissolved in DMSO at 10 mM and serially diluted into water containing 10% DMSO. Diluted compounds at a volume of 10 μL were added to wells of a 96-well V-bottomed microplate. A solution of DMEM, 1% glutamine, 1% penicillin and streptomycin, and 22 nM tritiated cortisone was prepared and 90 μL added to each well of the assay plate. This solution (100 μL/well) was transferred to the plate containing the cells. The plate was then incubated in 5% CO₂, 95% O₂ at 37° C. for 2 hours.

Following this incubation, 50 μL of the assay solution was transferred to each well of a 96-well scintillation microplate. A mixture consisting of anti-mouse YSi SPA beads, pre-mixed with anti-cortisol antibody in assay buffer (50 mM Tris.HCl, pH 7.0; 300 mM NaCl; 1 mM EDTA, 5% glycerol) was prepared and 50 μL added to each well of the scintillation microplate. An adhesive strip was applied to the microplate and the plate gently shaken for at least 2 hours at room temperature, and then spun briefly on a low speed centrifuge. The plate was read on a scintillation counter suitable for 96-well microplates. For the calculation of percentage inhibition, a series of wells were added to the plate that represented the assay maximum and the assay minimum: one set that contained substrate without cells (minimum) and another set that contained substrate and cells without any compound (maximum).

The calculation of median inhibitory concentration (IC₅₀) values for the compounds was performed using GraphPad Prism® software. Dose-response curves for each compound were plotted as fractional inhibition and data fitted to the four parameter logistic equation.

### Cellular In Vitro 11β-HSD2 Enzyme Inhibition Assay

For measurement of inhibition of 11β-HSD2, CHO cells stably transfected with the full-length gene coding for human 11β-HSD2 were used. Assays were carried out in 96-well microplates containing 1×10⁵ cells/well. Controls and compounds were plated as above, so that the final DMSO concentration in each well was 1%. To initiate the assay, 90 μL of a solution of HAMS F-12 medium containing 1% glutamine, 1% penicillin and streptomycin, and 22 nM tritiated cortisol was added to each well of the assay plate. The plate was then incubated in 5% CO₂, 95% O₂ at 37° C. for 16 hours.

The assay solutions were transferred to glass tubes and 20 μL ethyl acetate added to each tube. Each tube was vortexed thoroughly and the upper layer containing the tritiated steroid transferred to a fresh glass tube. The solvent was evaporated by placing the tubes in a heating block at 65° C. under a stream of Nitrogen gas. 20 μL ethanol was added to each of the dried samples and vortexed briefly. Each sample was applied to a silica TLC plate and the plate dried. The plate was placed vertically in a glass tank containing 92% chloroform:8% ethanol and the solvent allowed to rise up the plate. The plate was dried, placed in an imaging cassette, and overlayed with a tritium imaging plate for 1-2 days. The amount of enzyme inhibition in each sample was determined by measuring the intensity of the substrate and product spots using a phosphoimager.

IC₅₀ values for inhibitors were determined as described for 11β-HSD1.

### Biological Data

### Cellular In Vitro Enzyme Inhibition Data

The following compounds were tested using the cellular in vitro enzyme inhibition assays described above: AA-01, AA-02, BB-01, BB-02, BB-03, CC-01, CC-02.

Preferably, the compounds selectively inhibit 11β-HSD1 (i.e., have a low value for the IC₅₀ for 11β-HSD1) as compared to 11β-HSD2 (i.e., have a high value for the IC₅₀ for 11β-HSD2).

All of the compounds tested have an IC₅₀ for 11β-HSD1 of less than about 3 μM, and in most cases less than about 0.5 μM. All of the compounds tested have an IC₅₀ for 11β-HSD2 of more than 10,000 nM. All of the compounds tested have an IC₅₀ ratio for 11β-HSD2 to 11β-HSD1 of at least about five or greater, and in many cases ten or greater. For example, data for some of the compounds is shown in the following table.

TABLE 1
In vitro Enzyme Inhibition Data

| | IC₅₀ for 11β-HSD1 | IC₅₀ for 11β-HSD2 |
|---|---|---|
| Code No. | (HEK293) | (CHO) |
| AA-01 | 59 nM | >10,000 nM |
| BB-01 | 18 nM | >10,000 nM |
| CC-01 | 62 nM | >10,000 nM |

The following compounds have an IC₅₀ for 11β-HSD1 of less than or equal to 500 nM (0.5 μM), and an IC₅₀ for 11β-HSD2 of >10,000 nM: AA-01, AA-02, BB-01, BB-02, BB-03, CC-01.

The foregoing has described the principles, preferred embodiments, and modes of operation of the present invention. However, the invention should not be construed as limited to the particular embodiments discussed. Instead, the above-described embodiments should be regarded as illustrative rather than restrictive, and it should be appreciated that variations may be made in those embodiments by workers skilled in the art without departing from the scope of the present invention.

### REFERENCES

A number of publications are cited herein in order to more fully describe and disclose the invention and the state of the art to which the invention pertains. Full citations for these references are provided below. Each of these references is incorporated herein by reference in its entirety into the present disclosure, to the same extent as if each individual reference was specifically and individually indicated to be incorporated by reference.
Andrews, R. C., et al., 2003, “Effects of the llbeta-hydroxysteroid dehydrogenase inhibitor carbenoxolone on insulin sensitivity in men with type 2 diabetes,” J. Clin. Endocrinol. Metab., Vol. 88, pp. 285-291.
Arnold, W. D., et al., 2007, “Pyrimidinyl-Thiophene Kinase Modulators”, international patent publication number WO 2007/053776 A1 published 10 May 2007.
Bao, J., et al., 2000, “3-Thienyl and 3-Furanyl Pyrrolidine Modulators of Chemokine Receptor Activity”, international patent publication number WO 00/51608 A1 published 8 Sep. 2000.
Bystrom, S., et al., 2009, “Isoxazole derivatives as modulators of 11-beta-hydroxysteroid dehydrogenase type 1”, International Patent Application Publication No WO 2009/020239 A1 published 23 Jul. 2009.
Christy, C., et al., 2003, “Glucocorticoid action in mouse aorta; localisation of 11β-hydroxysteroid dehydrogenase type 2 and effects on responses to glucocorticoids in vitro,” Hypertension, Vol. 42, pp. 580-587.
Cooper, M. S., et al., 2000, “Expression and functional consequences of 11β-hydroxysteroid dehydrogenase activity in human bone,” Bone, Vol. 27, pp. 375-381.
Ernst, G., et al., 2004, “Biaryl Diazabicycloalkane Amides as Nicotinic Acetylcholine Agonists”, international patent publication number WO 2004/016617 A1 published 26 Feb. 2004.
Fotsch, C., et al., 2008, “Substituted azole aromatic heterocycles as inhibitors of 11β-HDS-1”, International Patent Application Publication No WO 2008/011453 A2 published 24 Jan. 2008.
Gotthardt, H., 1971, “1.3-Dipolare Cycloadditionen Mit 1.3.2-Oxathiazoliurn-5-oxiden. Ein Neuer Weg in Die 5-Aryl-Isothiazol-Reihe”, Tetrahedron Letters, No. 17, pp. 1281-1284.
Hadoke, P. W. F., et al., 2001, “Endothelial cell dysfunction in mice after transgenic knockout of type 2, but not type 1, 11β-hydroxysteroid dehydrogenase,” Circulation, Vol. 104, pp. 2832-2837.
Kotelevtsev, Y. V., et al., 1997, “11β-Hydroxysteroid dehydrogenase type 1 knockout mice show attenuated glucocorticoid inducible responses and resist hyperglycaemia on obesity and stress,” Proc. Natl. Acad. Sci., Vol. 94, pp. 14924-14929
Masuzaki, H., et al., 2001, “A Transgenic Model of Visceral Obesity and the Metabolic Syndrome,” Science, Vol. 294, pp. 2166-2170.
Moisan, M. P., et al., 1990, “11 beta-hydroxysteroid dehydrogenase bioactivity and messenger RNA expression in rat forebrain: localization in hypothalamus, hippocampus, and cortex,” Endocrinology, Vol. 127, pp. 1450-1455.
Morton, N. M., et al., 2001, “Improved lipid and lipoprotein profile, hepatic insulin sensitivity, and glucose tolerance in 11β-hydroxysteroid dehydrogenase type 1 null mice,” J. Biol. Chem., Vol. 276, pp. 41293-41300.
Morton, N. M., et al., 2004, “Novel adipose tissue-mediated resistance to diet-induced visceral obesity in 11β-hydroxysteroid dehydrogenase type 1 deficient mice,” Diabetes, Vol. 53, pp. 931-938.
Ogawa, et al., 2006, “Heterocyclic Compound Having Type I 11 Beta Hydroxysteroid Dehydrogenase Inhibitory Activity”, International Patent Application Publication No WO 2006/132197 published 14 Dec. 2006.
Ogawa, et al., 2008, “Heterocyclic Compound Having Type I 11 Beta Hydroxysteroid Dehydrogenase Inhibitory Activity”, European Patent Publication No EP 1 894 919 A1 published 5 Mar. 2008.
Paterson, J. M., et al., 2004, “Metabolic syndrome without obesity: hepatic overexpression of 11β-hydroxysteroid dehydrogenase type 1 in transgenic mice,” Proc. Natl. Acad. Sci., Vol. 101, pp. 7088-7093).
Rask, E., et al., 2001, “Tissue-specific dysregulation of cortisol metabolism in human obesity,” J. Clin. Endocrinol. Metab., Vol. 86, pp. 1418-1421.
Rauz, S., et al., 2001, “Expression and putative role of 11 beta-hydroxysteroid dehydrogenase isozymes within the human eye,” Investigative Opthalmology & Visual Science, Vol. 42, pp. 2037-2042.
Sandeep, T. C., et al., 2004, “11β-hydroxysteroid dehydrogenase inhibition improves cognitive function in healthy elderly men and type 2 diabetics,” Proc. Natl. Acad. Sci., Vol. 101, pp. 6734-6739.
Seckl, J. R., Walker, B.R., 2001, “11β-Hydroxysteroid dehydrogenase type 1-a tissue-specific amplifier of glucocorticoid action,” Endocrinology, Vol. 142, pp. 1371-1376.
Shaffer, J. E., et al., 1992, “Oxathi(siv)azol-5-one Compounds”, U.S. Pat. No. 5,087,631 granted 11 Feb. 1992.
Small, G. R., et al., 2005, “Preventing local regeneration of glucocorticoids by 11β-hydroxysteroid dehydrogenase type 1 enhances angiogenesis,” Proc. Natl. Acad. Sci., Vol. 102, pp. 12165-12170.
Walker, B. R., et al., 1991, “11β-Hydroxysteroid dehydrogenase in vascular smooth muscle and heart: implications for cardiovascular responses to glucocorticoids,” Endocrinology, Vol. 129, pp. 3305-3312.
Walker, B. R., et al., 1995, “Carbenoxolone increases hepatic insulin sensitivity in man: a novel role for 11-oxosteroid reductase in enhancing glucocorticoid receptor activation,” J. Clin. Endocrinol. Metab., Vol. 80, pp. 3155-3139.
Yau, J. L. W., et al., 2001, “Lack of tissue glucocorticoid reactivation in 11β-hydroxysteroid dehydrogenase type 1 knockout mice ameliorates age-related learning impairments,” Proc. Natl. Acad. Sci., Vol. 98, pp. 4716-4721.

1-207. (canceled)

208. A compound selected from compounds of the following formulae, and pharmaceutically acceptable salts thereof: 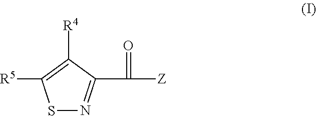
wherein:
—R⁴ is independently —H, —R^{4A}, or —R^{4B};
—R⁵ is independently —R^{5A1}, —R^{5B1}, or —R^{5B2};
—Z is independently -J¹ or -J²;
wherein:
—R^{4A} is independently saturated aliphatic C₁₋₄alkyl;
—R^{4B} is independently —F, —Cl or —Br;
—R^{5A1} is independently phenyl or naphthyl, and is optionally substituted;
—R^{5B1} is independently C₅₋₁₀heteroaryl, and is optionally substituted;
—R^{5B2} is independently non-aromatic C₄₋₇heterocyclyl, and is optionally substituted;
-J¹ is independently a monocyclic non-aromatic heterocyclyl group having from 4 to 8 ring atoms, wherein exactly 1 of said ring atoms is a ring heteroatom, and is N, or exactly 2 of said ring atoms are ring heteroatoms, and are both N, or exactly 2 of said ring atoms are ring heteroatoms, and are N and O, or exactly 2 of said ring atoms are ring heteroatoms, and are N and S, and wherein said non-aromatic heterocyclyl group is optionally substituted, and wherein -J¹ is attached via one of said ring atoms which is N; and
-J² is independently a fused bicyclic non-aromatic heterocyclyl group having from 7 to 12 ring atoms, wherein exactly 1 of said ring atoms is a ring heteroatom, and is N, or exactly 2 of said ring atoms are ring heteroatoms, and are both N, or exactly 2 of said ring atoms are ring heteroatoms, and are N and O, or exactly 2 of said ring atoms are ring heteroatoms, and are N and S, or exactly 3 of said ring atoms are ring heteroatoms, one of which is N, and each of the other two is independently N, O, or S, and wherein said fused bicyclic non-aromatic heterocyclyl group is optionally substituted, and wherein -J² is attached via one of said ring atoms which is N;
with the proviso that the compound is not a compound selected from (4-chloro-5-piperidin-1-yl-isothiazol-3-yl)-piperidin-1-yl-methanone and salts thereof;
wherein optional substituents on —R^{5A1}, and optional substituents on —R^{5B1}, and optional substituents on —R^{5B2} are independently selected from:
—R^{X1}, —CF₃,
—F, —Cl, —Br,
—OH, —OR^{X1}, —OCF₃,
—R^{XL}—OH, —R^{XL}—OR^{X1},
—CN,
—NO₂,
—NH₂, —NHR^{X1}, —NR^{X1}₂, -M,
—R^{XL}—NH₂, —R^{XL}—NHR^{X1}, —R^{XL}—NR^{X1}₂, —R^{XL}-M,
—NHC(═O)R^{X1}, —NR^{X1}C(═O)R^{X1},
—R^{XL}—NHC(═O)R^{X1}, —R^{XL}—NR^{X1}C(═O)R^{X1},
—C(═O)OH, —C(═O)OR^{X1},
—R^{XL}—C(═O)OH, —R^{XL}—C(═O)OR^{X1},
—C(═O)NH₂, —C(═O)NHR^{X1}, —C(═O)NR^{X1}₂, —C(═O)M,
—R^{XL}—C(═O)NH₂, —R^{XL}—C(═O)NHR^{X1}, —R^{XL}—C(═O)NR^{X1}₂, —R^{XL}—C(═O)M,
—S(═O)₂NH₂, —S(═O)₂NHR^{X1}, —S(═O)₂NR^{X1}₂, —S(═O)₂M,
—NHS(═O)₂R^{X1}, —NR^{X1}S(═O)₂R^{X1},
—NHC(═O)NH₂, —NHC(═O)NHR^{X1}, —NHC(═O)NR^{X1}₂, —NHC(═O)M,
—NR^{X1}C(═O)NH₂, —NR^{X1}C(═O)NHR^{X1}, —NR^{X1}C(═O)NR^{X1}₂, —NR^{X1}C(═O)M, and
═O;
or two adjacent substituents may together form —O—CH₂—O— or —O—CH₂CH₂—O—;
wherein:
each —R^{X1} is independently saturated aliphatic C₁₋₄ alkyl or phenyl;
each —R^{XL}— is independently saturated aliphatic C₁₋₄ alkylene; and
each -M is pyrrolidino, piperidino, piperazino, or morpholino, and is optionally substituted with one or more groups selected from saturated aliphatic C₁₋₄ alkyl;
and wherein optional substituents on -J¹, and optional substituents on -J², are independently selected from:
substituents on carbon, independently selected from —F, —OH, —OR^{X2}, —R^{X2}, —CH₂C(═O)OR^{X2}, —CF₃, —CN, phenyl, benzyl, thienyl, and pyridyl; and
substituents on nitrogen, independently selected from —R^{X2}, —CH₂CF₃, —S(═O)₂R^{X2} and —C(═O)R^{X2};
wherein each —R^{X2} is independently saturated aliphatic C₁₋₄ alkyl; and
wherein each phenyl, benzyl, thienyl, and pyridyl is optionally substituted with one or more groups selected from: —F, —Cl, —R^{X22}, —OH, —OR^{X22}, —CN, —NH₂, —NHR^{X22}, —NR^{X22}₂; wherein each —R^{X22} is independently saturated aliphatic C₁₋₄ alkyl.

209. A compound according to claim 208, wherein —R⁵ is independently —R^{5A1}.

210. A compound according to claim 208, wherein —R⁵ is independently —R^{5B1}.

211. A compound according to claim 208, wherein —R⁵ is independently —R^{5B2}.

212. A compound according to claim 208, wherein —Z is independently -J¹.

213. A compound according to claim 208, wherein —Z is independently -J².

214. A compound according to claim 208, wherein —R⁴ is independently —H.

215. A compound according to claim 209, wherein —R^{5A1} is independently phenyl, and is optionally substituted.

216. A compound according to claim 210, wherein —R^{5B1} is independently C₅₋₆heteroaryl, and is optionally substituted.

217. A compound according to claim 210, wherein —R^{5B1} is independently imidazolyl, pyrazolyl, oxazolyl, isoxazolyl, pyridyl, pyrimidinyl, or quinolinyl, and is optionally substituted.

218. A compound according to claim 210, wherein —R^{5B1} is independently pyrazolyl, and is optionally substituted.

219. A compound according to claim 210, wherein —R^{5B1} is independently pyrazol-4-yl, and is optionally substituted.

220. A compound according to claim 211, wherein —R^{5B2} is independently tetrahydropyranyl, and is optionally substituted.

221. A compound according to claim 211, wherein —R^{5B2} is independently tetrahydropyran-4-yl, and is optionally substituted.

222. A compound according to claim 212, wherein exactly 1 of said -J¹ ring atoms is a ring heteroatom, and is N.

223. A compound according to claim 212, wherein said -J¹ monocyclic non-aromatic heterocyclyl group has from 5 to 7 ring atoms.

224. A compound according to claim 212, wherein -J¹ is independently selected from the following groups and is optionally substituted:

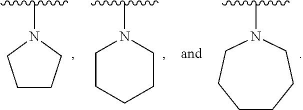

225. A compound according to claim 212, wherein -J¹ is independently:

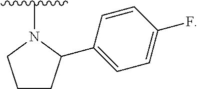

226. A compound according to claim 213, wherein exactly 1 of said -J² ring atoms is a ring heteroatom, and is N.

227. A compound according to claim 226, wherein said -J² fused bicyclic non-aromatic heterocyclyl group has 9 to 10 ring atoms.

228. A compound according to claim 213, wherein -J² is independently the following group and is optionally substituted:

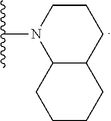

229. A compound according to claim 208, wherein optional substituents on —R^{5A1}, and optional substituents on —R^{5B1}, and optional substituents on —R^{5B2}, are independently selected from:
—R^{X1}, —F, —Cl, —Br, —OH, —OR^{X1}, —NH₂, —NHR^{X1}, —NR^{X1}₂, —NHC(═O)R^{X1}, —NR^{X1}C(═O)R^{X1}, and ═O;
wherein each —R^{X1} is independently saturated aliphatic C₁₋₄ alkyl or phenyl.

230. A compound according to claim 208, wherein optional substituents on -J¹, and optional substituents on -J², are independently selected from:
substituents on carbon, independently selected from phenyl, benzyl, thienyl, and pyridyl; and
substituents on nitrogen, independently selected from —R^{X2}, —CH₂CF₃, —S(═O)₂R^{X2} and —C(═O)R^{X2};
wherein each —R^{X2} is independently saturated aliphatic C₁₋₄ alkyl; and
wherein each phenyl, benzyl, thienyl, and pyridyl is optionally substituted with one or more groups selected from: —F, —Cl, —R^{X22}, —OH, —OR^{X22}, —CN, —NH₂, —NHR^{X22}, —NR^{X22}₂; wherein each —R^{X22} is independently saturated aliphatic C₁₋₄ alkyl.

231. A compound according to claim 208, selected from the following compounds, or a pharmaceutically acceptable salt thereof:

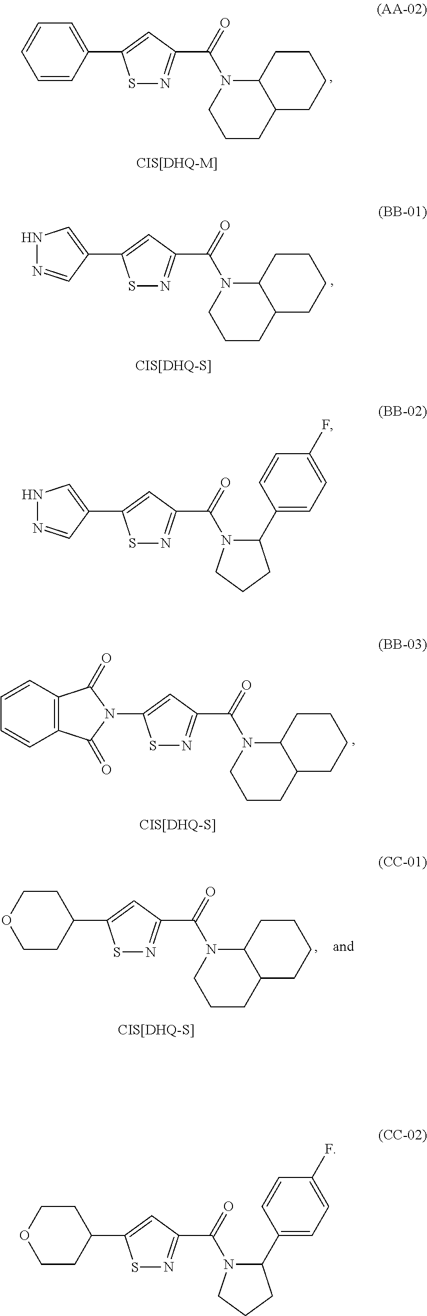

232. A pharmaceutical composition comprising a compound according to claim 208, and a pharmaceutically acceptable carrier or diluent.

233. A method of inhibiting 11β-hydroxysteroid dehydrogenase type 1 function in a cell, in vitro or in vivo, comprising contacting the cell with an effective amount of a compound according to claim 208, without the recited proviso regarding compound (PP-01).

234. A method of treatment or prevention of a disorder of the human or animal body that is ameliorated by the inhibition of 11β-hydroxysteroid dehydrogenase type 1 comprising administering to a subject in need of treatment a therapeutically-effective amount of a compound according to claim 208, without the recited proviso regarding compound (PP-01), wherein the disorder is:
(1) Cushing's syndrome;
(2) type 2 diabetes and impaired glucose tolerance;
(3) insulin resistance syndromes such as myotonic dystrophy, Prader Willi, lipodystrophies, gastrointestinal diabetes, etc;
(4) obesity and being overweight;
(5) lipid disorders;
(6) atherosclerosis and its sequelae, including myocardial infarction and peripheral vascular disease;
(7) Metabolic Syndrome;
(8) steatohepatitis/fatty liver;
(9) cognitive impairment in type 2 diabetes, glucose intolerance and ageing, and in psychotic disorders and pre-schizophrenia;
(10) dementias such as Alheimer's disease, multi-infarct dementia, dementia with Lewy bodies, fronto-temporal dementia (including Pick's disease), progressive supranuclear palsy, Korsakoffs syndrome, Binswanger's disease, HIV-associated dementia, Creutzfeldt-Jakob disease (CJD), multiple sclerosis, motor neurone disease, Parkinson's disease, Huntington's disease, Niemann-Pick disease type C, normal pressure hydrocephalus, and Down's syndrome;
(11) mild cognitive impairment (cognitive impairment, no dementia);
(12) β-cell dysfunction in pancreatic disease;
(13) glaucoma;
(14) anxiety;
(15) depression and other affective disorders; typical (melancholic) and atypical depression; dysthymia; post-partum depression; bipolar affective disorder; drug-induced affective disorders; anxiety; posttraumatic stress disorder; panic; phobias;
(16) delirium and acute confusional state;
(17) inflammatory disease;
(18) osteoporosis;
(19) myocardial infarction, for example, to prevent left ventricular dysfunction after myocardial infarction; or
(20) stroke, for example, to limit ischaemic neuronal loss after cardiovascular accident.

235. A method according to claim 234, wherein the disorder is:
(1) hyperglycaemia;
(2) glucose intolerance and impaired glucose tolerance;
(3) insulin resistance;
(4) hyperlipidaemia;
(5) hypertriglyceridaemia;
(6) hypercholesterolaemia;
(7) low HDL levels;
(8) high LDL levels;
(9) vascular restenosis;
(10) abdominal obesity;
(11) neurodegenerative disease;
(12) retinopathy;
(13) neuropathy;
(14) hypertension; or
(15) other diseases where insulin resistance is a component.

236. A method according to claim 234, wherein the disorder is:
an adverse effect of glucocorticoids used to treat inflammatory diseases, such as asthma, chronic obstructive pulmonary disease, skin diseases, rheumatoid arthritis and other arthropathies, inflammatory bowel disease, and giant cell arthritis/polymyalgia rheumatica; or
metabolic syndrome, which includes disorders such as type 2 diabetes and obesity, and associated disorders including insulin resistance, hypertension, lipid disorders and cardiovascular disorders such as ischaemic (coronary) heart disease; or
a CNS disorder such as mild cognitive impairment and early dementia, including Alzheimer's disease.
